(19) United States
(12) Patent Application Publication
(10) Pub. No.: US 2022/0144906 A1
(43) Pub. Date: May 12, 2022

(54) BIFUNCTIONAL BLOOD BRAIN THERAPIES FOR INTERLEUKIN-1 RELATED DISEASES

(71) Applicant: Bioasis Technologies, Inc., Burnaby (CA)

(72) Inventor: Mei Mei Tian, Maple Ridge (CA)

(21) Appl. No.: 17/587,427

(22) Filed: Jan. 28, 2022

Related U.S. Application Data
(63) Continuation-in-part of application No. 16/981,627, filed on Sep. 16, 2020.
(60) Provisional application No. 62/646,845, filed on Mar. 22, 2018.

(51) Int. Cl. C07K 14/47; A61P 25/00

(52) U.S. Cl. CPC C07K 14/4703; A61K 38/00; A61P 25/00

(57) ABSTRACT

Disclosed are therapeutic payloads comprising p97 fragments coupled with active agents having blood-brain barrier (BBB) transport activity, including variants and combinations thereof, to facilitate delivery of therapeutic or diagnostic agents across the BBB. The therapeutic payloads have dual functionality that may permit treatment of diseases in a subject other than diseases that present in the brain, e.g., solid tumors in the body. Methods of treating various diseases and pharmaceutical compositions are also disclosed.

### CROSS-REFERENCE TO RELATED APPLICATION

This application is a Continuation-in-Part of U.S. application Ser. No. 16/981,627 filed on Sep. 16, 2020, which is a national stage entry of PCT/US2019/023338 filed on Mar. 21, 2019, which further claims the benefit of U.S. Provisional Application No. 62/646,845, filed on Mar. 22, 2018.

### STATEMENT REGARDING THE SEQUENCE LISTING

The Sequence Listing associated with this application is provided in text format in lieu of a paper copy and is hereby incorporated by reference into the specification. The name of the text file containing the Sequence Listing is 20056PCT_ST25.txt. The text file is about 7 KB, was created on Jan. 28, 2022, and is being submitted electronically via EFS-Web.

### TECHNICAL FIELD OF THE INVENTION

The present invention relates to compounds for treating diseases, including compounds that penetrate the blood brain barrier. The invention also provides pharmaceutical compositions comprising compounds of the present invention and methods of using said compositions in the treatment of various disorders.

### BACKGROUND OF THE INVENTION

Overcoming the difficulties of delivering therapeutic agents to specific regions of the brain represents a major challenge to treatment or diagnosis of many central nervous system (CNS) disorders, including those of the brain. In its neuroprotective role, the blood-brain barrier (BBB) functions to hinder the delivery of many potentially important therapeutic agents to the brain.

Therapeutic agents that might otherwise be effective in diagnosis and therapy do not cross the BBB in adequate amounts. It is reported that over 95% of all therapeutic molecules do not cross the blood-brain barrier. Accordingly, it is desired to deliver therapeutic agents across the BBB to treat diseases.

In addition, therapeutic agents that may be effective intreating diseases in the brain may also be useful in treating diseases other than in the brain. Accordingly, it is desired to provide methods of treating diseases both in the brain of a subject and also outside the brain, such as for example, a solid tumor or hematological malignancy.

Moreover, interleukin-1 (IL-1) has been known as being associated with a number of diseases spanning from inflammatory diseases to cancers. Accordingly, it is desired to provide methods of treating diseases associated with IL-1 in both the brain of a subject and outside the brain of the subject.

### SUMMARY OF THE INVENTION

In accordance with the present invention, there are provided methods of treating diseases in the brain of a subject by administering a therapeutic payload that comprises an active agent coupled with a certain p97 fragment which enbles the active agent to cross the BBB. In accordance with the present invention, the therapeutic payload has pharmacokinetic properties that are similar to the active agent in a form that is uncoupled to the p97 fragment.

By virtue of the present invention, it is now possible to treat diseases in the brain of a subject and also treat diseases in the body (other than in the brain) or blood of the subject.

In one aspect of the invention, there is provided a method for treating a first interleukin-1 (IL-1) related disease in the brain of a subject by delivering a therapeutic payload across the blood-brain barrier of the subject, comprising administering to the subject said therapeutic payload, wherein said therapeutic payload comprises (i) an IL-1 receptor antagonist; (ii) a p97 fragment comprising an amino acid sequence at least 80% identical to DSSHAFTLDELR (SEQ ID NO: 2) coupled to the IL-1 receptor antagonist.

In one aspect, the method further comprises treating a second IL-1 related disease other than in the brain of the subject.

In one aspect, the therapeutic payload is administered to the subject other than intracranially.

In one aspect, the therapeutic payload is administered by oral, intravenous, intramuscular, subcutaneous, injection or infusion.

In one aspect, the first and second IL-1 related disease are selected from a group comprising autoinflammatory diseases, autoimmune disease, metabolic syndromes, chronic inflammation and malignancy, infectious diseases, ischemic diseases, fatigues associated with inflammatory and non-inflammatory diseases, neuropathic pain, fabry disease, epilepsy and seizure, fabrile disease, fabrile infection-related epilepsy syndrome, fabrile status epilepticus, generalised epilepsy with febrile seizures plus, dravet syndrome, Idiopathic hemiconvulsion hemiplegia and epilepsy syndrome, Parkinson's disease, IL-1 receptor antagonist deficiency syndrome, and new-onset refractory status epilepticus.

In one aspect, the therapeutic payload provides an AUCₗₐₛₜ (day·μg/mL) of greater than about 76% of the AUCₗₐₛₜ of the active agent in an uncoupled form.

In one aspect, the therapeutic payload provides an AUCₗₐₛₜ (day·μg/mL) of from about 77% to 150% of the AUCₗₐₛₜ of the active agent in an uncoupled form.

In one aspect, the therapeutic payload provides an AUCₗₐₛₜ (day·μg/mL) of from about 80% to 125% of the AUCₗₐₛₜ of the active agent in an uncoupled form.

In one aspect, the IL-1 receptor antagonist comprises at least one of anakinra, canakinumab, or rilonacept.

In one aspect of the invention, there is provided a method of delivering a therapeutic payload across the blood-brain barrier of a subject, comprising administering to the subject said therapeutic payload, wherein said therapeutic payload; (i) comprises an active agent coupled with a p97 fragment comprising an amino acid sequence at least 80% identical to DSSHAFTLDELR (SEQ ID NO: 2); and (ii) provides an AUCₗₐₛₜ (day·μg/mL) of greater than about 76% of the AUCₗₐₛₜ of the active agent in an uncoupled form.

In one aspect, the therapeutic payload provides an AUCₗₐₛₜ (day·μg/mL) of from about 77% to 150% of the AUCₗₐₛₜ of the active agent in an uncoupled form. In one aspect, the therapeutic payload provides an AUCₗₐₛₜ (day·μg/mL) of from about 80% to 125% of the AUCₗₐₛₜ of the active agent in an uncoupled form.

In one aspect, the p97 fragment has one or more terminal cysteines and/or tyrosines.

In one aspect, the p97 fragment consists essentially of DSSHAFTLDELR (SEQ ID NO: 2) with a C-terminal tyrosine, and wherein the p97 fragment and the active agent are separated by a peptide linker of about 1-10 amino acids in length.

In one aspect, the p97 fragment consists essentially of DSSHAFTLDELR (SEQ ID NO: 2) with a C-terminal cysteine, and wherein the p97 fragment and the active agent are separated by a peptide linker of about 1-10 amino acids in length.

In one aspect, the the p97 fragment consists essentially of DSSHAFTLDELR (SEQ ID NO: 2) with a N-terminal tyrosine, and wherein the p97 fragment and the active agent are separated by a peptide linker of about 1-10 amino acids in length.

In one aspect, the p97 fragment consists essentially of DSSHAFTLDELR (SEQ ID NO: 2) with a N-terminal cysteine, and wherein the p97 fragment and the active agent are separated by a peptide linker of about 1-10 amino acids in length.

In one aspect, the active agent is a small molecule, a polypeptide, a peptide mimetic, a peptoid, an aptamer, or a detectable entity.

In one aspect, the small molecule is a cytotoxic or chemotherapeutic or anti-angiogenic agent selected from one or more of alkylating agents, anti-metabolites, anthracyclines, anti-tumor antibiotics, platinums, type I topoisomerase inhibitors, type II topoisomerase inhibitors, vinca alkaloids, and taxanes.

In one aspect, the polypeptide is an antibody or antigen-binding fragment thereof.

In one aspect, the the antibody or antigen-binding fragment thereof specifically binds to a cancer-associated antigen.

In one aspect of the invention, there is provided a method of delivering a therapeutic payload across the blood-brain barrier of a subject, comprising administering to the subject said therapeutic payload, wherein said therapeutic payload; (i) comprises an active agent coupled with a p97 fragment comprising an amino acid sequence at least 80% identical to DSSHAFTLDELR (SEQ ID NO: 2); and (ii) provides a Vz (ml/kg) of less than about 173% of the Vz of the active agent in an uncoupled form.

In one aspect, the therapeutic payload provides a Vz (ml/kg) of from about 50% to 150% of the Vz of the active agent in an uncoupled form.

In one aspect, the therapeutic payload provides a Vz (ml/kg) of from about 80% to 125% of the Vz of the active agent in an uncoupled form.

In one aspect of the invention, there is provided a method of delivering a therapeutic payload across the blood-brain barrier of a subject, comprising administering to the subject said therapeutic payload, wherein said therapeutic payload; (i) comprises an active agent coupled with a p97 fragment comprising an amino acid sequence at least 80% identical to DSSHAFTLDELR (SEQ ID NO: 2); and (ii) provides a CL (ml/day/kg) of less than 178% of the CL of the active agent in an uncoupled form.

In one aspect, the therapeutic payload provides a CL (ml/day/kg) of from about 50% to 176% of the CL of the active agent in an uncoupled form.

In one aspect, the therapeutic payload provides a CI (ml/day/kg) of from about 80% to 125% of the CL of the active agent in an uncoupled form.

In one aspect of the invention, there is provided a method of binding a therapeutic payload to an LRP1 receptor in a subject, comprising contacting the LRP1 receptor with said therapeutic payload, wherein said therapeutic payload; (i) comprises an active agent coupled with a p97 fragment consisting essentially of DSSHAFTLDELR (SEQ ID NO: 2); and (ii) provides an AUCₗₐₛₜ (day·μg/mL) of greater than about 76% of the AUCₗₐₛₜ of the active agent in an uncoupled form.

In one aspect of the invention, there is provided a method of treating a first disease in the brain of a subject by delivering a therapeutic payload across the blood-brain barrier of the subject, comprising administering to the subject said therapeutic payload, wherein said therapeutic payload; (i) comprises an active agent coupled with a p97 fragment comprising an amino acid sequence at least 80% identical to DSSHAFTLDELR (SEQ ID NO: 2); and (ii) provides an AUCₗₐₛₜ (day·μg/mL) of greater than about 76% of the AUCₗₐₛₜ of the active agent in an uncoupled form.

In one aspect, the therapeutic payload provides an AUCₗₐₛₜ (day·μg/mL) of from about 77% to 150% of the AUCₗₐₛₜ of the active agent in an uncoupled form.

In one aspect, the therapeutic payload provides an AUCₗₐₛₜ (day·μg/mL) of from about 80% to 125% of the AUCₗₐₛₜ of the active agent in an uncoupled form.

In one aspect, the invention further comprises treating a second disease other than in the brain of the subject.

In one aspect, the therapeutic payload is administered to the subject other than intracranially.

In one aspect, the therapeutic payload is administered by oral, intravenous, intramuscular, subcutaneous, injection or infusion.

In one aspect, the first disease and the second disease are the same.

In one aspect, the first disease and the second disease are different.

In one aspect, the the first disease presents in the form of a tumor or abnormality in the brain of the subject.

In one aspect, the second disease presents in the form of a tumor or abnormality in the body or blood of the subject other than in the brain.

In one aspect, the p97 fragment comprises an amino acid sequence at least 80% identical to DSSHAFTLDELR (SEQ ID NO: 2).

In one aspect, the p97 fragment comprises an amino acid sequence at least 85% identical to DSSHAFTLDELR (SEQ ID NO: 2).

In one aspect, the p97 fragment comprises an amino acid sequence at least 90% identical to DSSHAFTLDELR (SEQ ID NO: 2).

In one aspect, the p97 fragment comprises an amino acid sequence at least 95% identical to DSSHAFTLDELR (SEQ ID NO: 2).

In one aspect, the p97 fragment comprises an amino acid sequence that is 100% identical to DSSHAFTLDELR (SEQ ID NO: 2).

In one aspect, the p97 fragment comprises an amino acid sequence wherein one amino acid residue is different that the sequence DSSHAFTLDELR (SEQ ID NO: 2).

In one aspect, the p97 fragment comprises an amino acid sequence wherein two amino acid residues are different that the sequence DSSHAFTLDELR (SEQ ID NO: 2).

### BRIEF DESCRIPTION OF THE DRAWINGS

FIGS. 1A and 1B show a schematic of the different molecules studied. FIG. 1A shows the constructs with NIP228 hIgG1TM antibody alone and incorporating as genetic fusion with flexible linker or chemical conjugation MTf and MTfpep. FIG. 1B shows the constructs with NIP228hIgGTM antibody or the Fc fragment of the antibody containing the therapeutic molecule IL-1RA (Kineret) with an analgesic effect and incorporating after genetic fusion MTf or MTfpep with a flexible linker.

FIGS. 2A-2D show representative 3D confocal image of brain distribution of different constructs based on the mAb NIP228 an hIgG1TM 2 hrs after IV administration in CD-1 female mice.

FIG. 2A shows the distribution of the NIP228 labelled with Alexa F647 (red). FIG. 2B shows the distribution of MTf chemically conjugated to NIP228 labelled with Alexa F647 (red). FIG. 2C shows the distribution of MTfpep chemically conjugated to NIP228 labelled with Alexa F647 (red). FIG. 2D shows magnified surface rendered (quantified) Texas Red labelled blood capillaries (green) and MTfpep chemically conjugated to NIP228 labelled with Alexa F647 (red).

FIG. 3 shows confocal fluorescence microscopy analysis and semi-quantification of the distribution of the different molecules in the brain parenchyma.

FIGS. 4A-4C show plasma and brain exposure of MTf or MTfpep targeted IgGs in a mouse PK assay. FIG. 4A shows plasma PK of MTf or MTfpep targeted hIgG1TM compared to a non-targeted isotype control (NIP228) over a two-week period. FIG. 4B shows brain exposure as a measure of % injected dose per gram of brain. FIG. 4C shows a comparison of brain:plasma ratio.

FIGS. 5A and 5B show plasma and brain exposure of MTf and MTfpep targeted IgG-IL-1RA fusion molecules in a mouse PK assay. FIG. 5A shows plasma PK of MTf and MTfpep targeted hIgG fused to IL-1RA compared to a non-targeted isotype control (NIP228) over a two-week period. FIG. 5B shows brain exposure as a measure of % injected dose per gram of brain.

FIGS. 6A and 6B show the the analgesic effect of MTf-IL-1RA fusions on the mouse partial nerve ligation model. FIG. 6A shows a comparison of the analgesic effect of IL-1RA fusion constructs containing MTf and MTfpep with isotype control (NIP228), vehicle control and non-ligated (sham operated) control group. FIG. 6B shows a dose response of MTfpep targeted NIP228 higG1TM-IL-1RA fusions on the reversal of partial nerve ligation-induced mechanical hyperalgesia.

FIGS. 7A and 7B show sequence differences in the amino acid sequence of the peptide of SEQ ID NO: 2 (referred to as “xB³”) and the transport of the peptides across the BBB. FIG. 7A shows sequence differences in the amino acid sequence of the peptide of SEQ ID NO: 2. FIG. 7B shows a comparison of the transport of the peptides across the BBB.

### DETAILED DESCRIPTION OF CERTAIN EMBODIMENTS

The following detailed description is provided to aid those skilled in the art in practicing the present invention. Those of ordinary skill in the art may make modifications and variations in the embodiments described herein without departing from the spirit or scope of the present disclosure.

Unless otherwise defined, all technical and scientific terms used herein have the same meaning as commonly understood by one of ordinary skill in the art to which this disclosure belongs. The terminology used in the description is for describing particular embodiments only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “administering” refers to the physical introduction of a composition comprising a therapeutic agent to a subject, using any of the various methods and delivery systems known to those skilled in the art. For example, routes of administration can include bucal, intranasal, ophthalmic, oral, osmotic, parenteral, rectal, sublingual, topical, transdermal, vaginal intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods and can be a therapeutically effective dose or a subtherapeutic dose.

The term “AUC” (area under the curve) refers to a total amount of drug absorbed or exposed to a subject. Generally, AUC may be obtained from mathematical method in a plot of drug concentration in the subject over time until the concentration is negligible. The term “AUC” (area under the curve) could also refer to partial AUC at specified time intervals (as may be the case with sublingual absorption which would increase AUC at earlier time intervals).

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as thee-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

The term “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” includes primary, metastatic and recurrent cancers as well as a precancerous condition, i.e., a state of disordered morphology of cells that is associated with an increased risk of cancer. The term “cancer” includes, but is not limited to, the following proliferative diseases: Acute Lymphoblastic Leukemia (ALL), Acute Myeloid Leukemia (AML), Adrenocortical Carcinoms, Childhood cancers, AIDS-Related Cancers, Kaposi Sarcoma, AIDS-Related Lymphoma, Primary CNS Lymphoma, Anal Cancer, Astrocytomas, Atypical Teratoid/Rhabdoid Tumor, Basal Cell Carcinoma, Skin Cancer (Nonmelanoma), Bile Duct Cancer, Bladder Cancer, Bone Cancer, Ewing Sarcoma Family of Tumors, Osteosarcoma and Malignant Fibrous Histiocytoma, Brain Stem Glioma, Atypical Teratoid/Rhabdoid Tumor, Embryonal Tumors, Germ Cell Tumors, Craniopharyngioma, Ependymoma, Breast Cancer, Bronchial Tumors, Burkitt Lymphoma, Non-Hodgkin Lymphoma, Carcinoid Tumor, Gastrointestinal Carcinoma, Cardiac (Heart) Tumors, Primary Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Mycosis Fungoides and Sezary Syndrome, Ductal Carcinoma In Situ (DCIS), Embryonal Tumors, Endometrial Cancer, Ependymoma, Esophageal Cancer, Esthesioneuroblastoma, Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumor, Ovarian, Testicular, Gestational Trophoblastic Disease, Glioma, Hairy Cell Leukemia, Head and Neck Cancer, Hepatocellular (Liver) Cancer, Histiocytosis, Langerhans Cell, Hodgkin Lymphoma, Hypopharyngeal Cancer, Islet Cell Tumors, Pancreatic Neuroendocrine Tumors, Kaposi Sarcoma, Kidney, Renal Cell, Langerhans Cell Histiocytosis, Laryngeal Cancer, Leukemia, Acute Lymphoblastic (ALL), Acute Myeloid (AML), Chronic Lymphocytic (CLL), Chronic Myelogenous (CML), Hairy Cell, Lip and Oral Cavity Cancer, Liver Cancer (Primary), Lung Cancer, Non-Small Cell, Small Cell, Lymphoma, Hodgkin, Non-Hodgkin, Macroglobulinemia, Waldenström, Male Breast Cancer, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML) Myeloma, Multiple, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Ovarian Cancer, Low Malignant Potential Tumor, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary CNS Lymphoma, Primary Peritoneal Cancer, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Rhabdomyosarcoma, Uterine, Small Intestine Cancer, Soft Tissue Sarcoma, Sqamous Cell Carcinoma, Squamous Neck Cancer with Occult Primary, Metastatic, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Unknown Primary, Ureter and Renal Pelvis, Transitional Cell Cancer, Urethral Cancer, Uterine Cancer, Endometrial, Uterine Sarcoma, Vaginal Cancer, Vulvar Cancer, Waldenström Macroglobulinemia, and Wilms Tumor.

The term “Cmax” refers to a maximum concentration of a drug in blood, serum, a specified compartment or test area of a subject between administration of a first dose and administration of a second dose. The term Cmax could also refer to dose normalized ratios if specified.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of. “Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “conjugate” is intended to refer to the entity formed as a result of covalent or non-covalent attachment or linkage of an agent or other molecule, e.g., a biologically active molecule, to a p97 polypeptide. One example of a conjugate polypeptide is a “fusion protein” or “fusion polypeptide,” that is, a polypeptide that is created through the joining of two or more coding sequences, which originally coded for separate polypeptides; translation of the joined coding sequences results in a single, fusion polypeptide, typically with functional properties derived from each of the separate polypeptides.

As used herein, the terms “function” and “functional” and the like refer to a biological, enzymatic, or therapeutic function.

“Homology” refers to the percentage number of amino acids that are identical or constitute conservative substitutions. Homology may be determined using sequence comparison programs such as GAP (Deveraux et al., Nucleic Acids Research. 12, 387-395, 1984), which is incorporated herein by reference. In this way sequences of a similar or substantially different length to those cited herein could be compared by insertion of gaps into the alignment, such gaps being determined, for example, by the comparison algorithm used by GAP.

By “isolated” is meant material that is substantially or essentially free from components that normally accompany it in its native state. For example, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances.

The term “linkage,” “linker,” “linker moiety,” or “L” is used herein to refer to a linker that can be used to separate a p97 polypeptide fragment from an agent of interest, or to separate a first agent from another agent, for instance where two or more agents are linked to form a p97 conjugate. The linker may be physiologically stable or may include a releasable linker such as an enzymatically degradable linker (e.g., proteolytically cleavable linkers). In certain aspects, the linker may be a peptide linker, for instance, as part of a p97 fusion protein. In some aspects, the linker may be a non-peptide linker or non-proteinaceous linker. In some aspects, the linker may be particle, such as a nanoparticle.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (e.g., the absence of polypeptide of conjugate of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition or a control composition, including all integers in between. As one non-limiting example, a control could compare the activity, such as the amount or rate of transport/delivery across the blood brain barrier, the rate and/or levels of distribution to central nervous system tissue, and/or the Cmax for plasma, central nervous system tissues, or any other systemic or peripheral non-central nervous system tissues, of a p97-agent conjugate relative to the agent alone. Other examples of comparisons and “statistically significant” amounts are described herein.

In certain embodiments, the “purity” of any given agent (e.g., a p97 conjugate such as a fusion protein) in a composition may be specifically defined. For instance, certain compositions may comprise an agent that is at least 80%, 85%, 90%, 91%, 92%, 93%, 94%, 95%, 96%, 97%, 98%, 99%, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations and the like, as well as other modifications known in the art, both naturally occurring and non-naturally occurring. A polypeptide may be an entire protein, or a subsequence, fragment, variant, or derivative thereof.

A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include, but are not limited to: carboxylate ester, phosphate ester, anhydride, acetal, ketal, acyloxyalkyl ether, imine, orthoester, thio ester, thiol ester, carbonate, and hydrazone, peptides and oligonucleotides.

A “releasable linker” includes, but is not limited to, a physiologically cleavable linker and an enzymatically degradable linker. Thus, a “releasable linker” is a linker that may undergo either spontaneous hydrolysis, or cleavage by some other mechanism (e.g., enzyme-catalyzed, acid-catalyzed, base-catalyzed, and so forth) under physiological conditions. For example, a “releasable linker” can involve an elimination reaction that has a base abstraction of a proton, (e.g., an ionizable hydrogen atom, Ha), as the driving force. For purposes herein, a “releasable linker” is synonymous with a “degradable linker.” An “enzymatically degradable linkage” includes a linkage, e.g., amino acid sequence that is subject to degradation by one or more enzymes, e.g., peptidases or proteases. In particular embodiments, a releasable linker has a half life at pH 7.4, 25° C., e.g., a physiological pH, human body temperature (e.g., in vivo), of about 30 minutes, about 1 hour, about 2 hour, about 3 hours, about 4 hours, about 5 hours, about 6 hours, about 12 hours, about 18 hours, about 24 hours, about 36 hours, about 48 hours, about 72 hours, or about 96 hours or less.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length.

Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted, for example, by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997. A detailed discussion of sequence analysis can be found in Unit 19.3 of Ausubel et al., “Current Protocols in Molecular Biology,” John Wiley & Sons Inc, 1994-1998, Chapter 15.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

The term “solubility” refers to the property of a p97 polypeptide fragment or conjugate to dissolve in a liquid solvent and form a homogeneous solution. Solubility is typically expressed as a concentration, either by mass of solute per unit volume of solvent (g of solute per kg of solvent, g per dL (100 ml), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, or pH 7.4. In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and IOmM NaP). In certain embodiments, solubility is measured in a biological fluid (solvent) such as blood or serum. In certain embodiments, the temperature can be about room temperature (e.g., about 20, 21, 22, 23, 24, 25° ( ) or about body temperature (−37° C.). In certain embodiments, a p97 polypeptide or conjugate has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 mg/ml at room temperature or at about 37° C.

A “subject,” as used herein, includes any animal that exhibits a symptom, or is at risk for exhibiting a symptom, which can be treated or diagnosed with a p97 conjugate of the invention. Suitable subjects (patients) include laboratory animals (such as mouse, rat, rabbit, or guinea pig), farm animals, and domestic animals or pets (such as a cat or dog). Non-human primates and, preferably, human patients, are included.

“Substantially” or “essentially” means nearly totally or completely, for instance, 95%, 96%, 97%, 98%, 99% or greater of some given quantity.

“Substantially free” refers to the nearly complete or complete absence of a given quantity for instance, less than about 10%, 5%, 4%, 3%, 2%, 1%, 0.5% or less of some given quantity. For example, certain compositions may be “substantially free” of cell proteins, membranes, nucleic acids, endotoxins, or other contaminants.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

The term “wild-type” refers to a gene or gene product that has the characteristics of that gene or gene product when isolated from a naturally-occurring source. A wild type gene or gene product (e.g., a polypeptide) is that which is most frequently observed in a population and is thus arbitrarily designed the “normal” or “wild-type” form of the gene.

p97 Polypeptide Sequences and Conjugates Thereof

Embodiments of the present invention relate generally to polypeptide fragments of human p97 (melanotransferrin; MTf), compositions that comprise such fragments, and conjugates thereof. In certain instances, the p97 polypeptide fragments described herein have transport activity, that is, they are ability to transport across the blood-brain barrier (BBB). In particular embodiments, the p97 fragments are covalently, non-covalently, or operatively coupled to an agent of interest, such as a therapeutic, diagnostic, or detectable agent, to form a p97-agent conjugate. Specific examples of agents include small molecules and polypeptides, such as antibodies, among other agents described herein and known in the art. Exemplary p97 polypeptide sequences and agents are described below. Also described are exemplary methods and components, such as linker groups, for coupling a p97 polypeptide to an agent of interest.

p97 Sequence. In some embodiments, a p97 polypeptide comprises, consists essentially of, or consists of the human p97 fragments identified in SEQ ID NO 2 (DSSHAFTLDELR).

In other specific embodiments, described in greater detail below, a p97 polypeptide sequence comprises a sequence having at least 70%, 75%, 80%, 85%, 90%, 95%, 96%, 97%, 98%, or 99% identity or homology, along its length, to the human p97 sequence set forth in SEQ ID NO 2. Typically, the p97 polypeptide comprises an amino acid sequence at least 80%, more typically at least 85%, even more typically at least 90%, even more typically at least 95% identical to DSSHAFTLDELR (SEQ ID NO:2). Often, the p97 polypeptide comprises an amino acid sequence that is 100% identical to DSSHAFTLDELR (SEQ ID NO:2). Typically, the p97 polypeptide comprises an amino acid sequence where 0, 1 or 2 amino acids are different from DSSHAFTLDELR (SEQ ID NO:2), e.g., DSSYSFTLDELR (SEQ ID NO: 3).

In particular embodiments, the p97 fragment or variant thereof has the ability to cross the BBB, and optionally transport an agent of interest across the BBB and into the central nervous system. In certain embodiments, the p97 fragment or variant thereof is capable of specifically binding to a p97 receptor, an LRPI receptor, and/or an LRPIB receptor.

In some embodiments, the p97 fragment has one or more terminal (e.g., N-terminal, C-terminal) cysteines and/or tyrosines, which can be added for conjugation and iodination, respectively.

Variants and fragments of reference p97 polypeptides and other reference polypeptides are described in greater detail below.

P97 Couplings. As noted above, certain embodiments comprise a p97 polypeptide that is coupled to an agent of interest, for instance, a small molecule, a polypeptide (e.g., peptide, antibody), a peptide mimetic, a peptoid, an aptamer, a detectable entity, or any combination thereof by fusion or conjugation. Also included are conjugates that comprise more than one agent of interest, for instance, a p97 fragment conjugated to an antibody and a small molecule.

Covalent linkages are preferred, however, non-covalent linkages can also be employed, including those that utilize relatively strong non-covalent protein-ligand interactions, such as the interaction between biotin and avidin. Fusion of the p97 fragment with the agent is especially preferred. Operative linkages are also included, which do not necessarily require a directly covalent or non-covalent interaction between the p97 fragment and the agent of interest; examples of such linkages include liposome mixtures that comprise a p97 polypeptide and an agent of interest. Exemplary methods of generating protein conjugates are described herein, and other methods are well-known in the art.

Small Molecules. In particular embodiments, the p97 fragment is conjugated to a small molecule. A “small molecule” refers to an organic compound that is of synthetic or biological origin (biomolecule), but is typically not a polymer. Organic compounds refer to a large class of chemical compounds whose molecules contain carbon, typically excluding those that contain only carbonates, simple oxides of carbon, or cyanides. A “biomolecule” refers generally to an organic molecule that is produced by a living organism, including large polymeric molecules (biopolymers) such as peptides, polysaccharides, and nucleic acids as well, and small molecules such as primary secondary metabolites, lipids, phospholipids, glycolipids, sterols, glycerolipids, vitamins, and hormones. A “polymer” refers generally to a large molecule or macromolecule composed of repeating structural units, which are typically connected by covalent chemical bond.

In certain embodiments, a small molecule has a molecular weight of less than about 1000-2000 Daltons, typically between about 300 and 700 Daltons, and including about 50, 100, 150, 200, 250, 300, 350, 400, 450, 500, 550, 500, 650, 600, 750, 700, 850, 800, 950, 1000 or 2000 Daltons.

Certain small molecules can have the “specific binding” characteristics described for antibodies (infra). For instance, a small molecule can specifically bind to a target described herein with a binding affinity (Kd) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In certain embodiments a small specifically binds to a cell surface receptor or other cell surface protein. In some embodiments, the small molecule specifically binds to at least one cancer-associated antigen described herein. In particular embodiments, the small molecule specifically binds to at least one nervous system-associated, pain-associated, and/or autoimmune-associated antigen described herein.

Exemplary small molecules include cytotoxic, chemotherapeutic, and anti-angiogenic agents, for instance, those that have been considered useful in the treatment of various cancers, including cancers of the central nervous system and cancers that have metastasized to the central nervous system.

Particular classes of small molecules include, without limitation, alkylating agents, anti-metabolites, anthracyclines, anti-tumor antibiotics, platinums, type I topoisomerase inhibitors, type II topoisomerase inhibitors, vinca alkaloids, and taxanes.

Specific examples of small molecules include chlorambucil, cyclophosphamide, cilengitide, lomustine (CCNU), melphalan, procarbazine, thiotepa, carmustine (BCNU), enzastaurin, busulfan, daunorubicin, doxorubicin, gefitinib, erlotinib idarubicin, temozolomide, epirubicin, mitoxantrone, bleomycin, cisplatin, carboplatin, oxaliplatin, camptothecins, irinotecan, topotecan, amsacrine, etoposide, etoposide phosphate, teniposide, temsirolimus, everolimus, vincristine, vinblastine, vinorelbine, vindesine, CT52923, and paclitaxel, and pharmaceutically acceptable salts, acids or derivatives of any of the above.

Additional examples of small molecules include those that target protein kinases for the treatment of nervous system (e.g., CNS) disorders, including imatinib, dasatinib, sorafenib, pazopanib, sunitnib, vatalanib, geftinib, erlotinib, AEE-788, dichoroacetate, tamoxifen, fasudil, SB-681323, and semaxanib (SU5416) (see Chico et al., Nat Rev Drug Discov. 8:829-909, 2009). Examples of small molecules also include donepizil, galantamine, memantine, rivastigmine, tacrine, rasigiline, naltrexone, lubiprostone, safinamide, istradefylline, pimavanserin, pitolisant, isradipine, pridopidine (ACR16), tetrabenazine, and bexarotene (e.g., for treating Alzheimer's Disease, Parkinson's Disease, Huntington's Disease); and glatirimer acetate, fingolimod, mitoxantrone (e.g., for treating MS). Also included are pharmaceutically acceptable salts, acids or derivatives of any of the above.

Further examples of small molecules include alkylating agents such as thiotepa, cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunoru bicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above.

Also included are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As noted above, in certain aspects the small molecule is an otherwise cardiotoxic agent.

Particular examples of cardiotoxic small molecules include, without limitation, anthracyclines/anthraquinolones, cyclophosphamides, antimetabolites, antimicrotubule agents, and tyrosine kinase inhibitors. Specific examples of cardiotoxic agents include cyclopentenyl cytosine, 5-fluorouracil, capecitabine, paclitaxel, docataxel, adriamycin, doxorubucin, epirubicin, emetine, isotamide, mitomycin C, erlotinib, gefitinib, imatinib, sorafenib, sunitinib, cisplatin, thalidomide, busulfan, vinblastine, bleomycin, vincristine, arsenic trioxide, methotrexate, rosiglitazone, and mitoxantrone, among other small molecules described herein and known in the art.

Polypeptide Agents. In particular embodiments, the agent of interest is a peptide or polypeptide. The terms “peptide” and “polypeptide” are used interchangeably herein, however, in certain instances, the term “peptide” can refer to shorter polypeptides, for example, polypeptides that consist of about 2, 3, 4, 5, 6, 7, 8, 9, 10, 11, 12, 13, 14, 15, 16, 17, 18, 19, 20, 25, 30, 35, 40, 45, or 50 amino acids, including all integers and ranges (e.g., 5-10, 8-12, 10-15) in between. Polypeptides and peptides can be composed of naturally-occurring amino acids and/or non-naturally occurring amino acids, as described herein. Antibodies are also included as polypeptides.

Exemplary polypeptide agents include polypeptides associated with lysosomal storage disorders. Examples of such polypeptides include aspartylglucosaminidase, acid lipase, cysteine transporter, Lamp-2, a-galactosidase A, acid ceramidase, a-L-fucosidase, -hexosaminidase A, GM2-ganglioside activator (GM2A), a-D-mannosidase, -D-mannosidase, arylsulfatase A, saposin B, neuraminidase, a-N-acetylglucosaminidase phosphotransferase, phosphotransferase y-subunit, L-iduronidase, iduronate-2-sulfatase, heparan-N-sulfatase, a-N-acetylglucosaminidase, acetylCoA:N-acetyltransferase, N-acetylglucosamine 6-sulfatase, galactose 6-sulfatase, -galactosidase, N-acetylgalactosamine 4-sulfatase, hyaluronoglucosaminidase, sulfatases, palmitoyl protein thioesterase, tripeptidyl peptidase I, acid sphingomyelinase, cathepsin A, cathepsin K, a-galactosidase B, NPCI, NPC2, sialin, and sialic acid transporter, including fragments, variants, and derivatives thereof.

Certain embodiments include polypeptides such as interferon-polypeptides, such as interferon-Ia (e.g., AVONEX, REBIF) and interferon-Ib (e.g., Betaseron), which are often used for the treatment of multiple sclerosis (MS).

In some embodiments, as noted above, the polypeptide agent is an antibody or an antigen-binding fragment thereof. The antibody or antigen-binding fragment used in the conjugates or compositions of the present invention can be of essentially any type. Particular examples include therapeutic and diagnostic antibodies. As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule.

As used herein, the term “antibody” encompasses not only intact polyclonal or monoclonal antibodies, but also fragments thereof (such as dAb, Fab, Fab′, F(ab′h, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity.

The term “antigen-binding fragment” as used herein refers to a polypeptide fragment that contains at least one CDR of an immunoglobulin heavy and/or light chains that binds to the antigen of interest. In this regard, an antigen-binding fragment of the herein described antibodies may comprise 1, 2, 3, 4, 5, or all 6 CDRs of a VH and VL sequence from antibodies that bind to a therapeutic or diagnostic target.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, capable of specific binding to an immunoglobulin or T-cell receptor. An epitope is a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. Epitopes can be contiguous or non-contiguous in relation to the primary structure of the antigen.

A molecule such as an antibody is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity.

Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd.

Immunological binding properties of selected antibodies and polypeptides can be quantified using methods well known in the art (see Davies et al., Annual Rev. Biochem. 59:439-473, 1990). In some embodiments, an antibody or other polypeptide is said to specifically bind an antigen or epitope thereof when the equilibrium dissociation constant is about ≤10⁻⁷ or 10⁻⁸ M. In some embodiments, the equilibrium dissociation constant of an antibody may be about ≤10⁻⁹ M or ≤10⁻¹⁰ M. In certain illustrative embodiments, an antibody or other polypeptide has an affinity (Kd) for an antigen or target described herein (to which it specifically binds) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the antibody or antigen-binding fragment or other polypeptide specifically binds to a cell surface receptor or other cell surface protein. In some embodiments, the antibody or antigen-binding fragment or other polypeptide specifically binds to a ligand of a cell surface receptor or other cell surface protein. In some embodiments, the antibody or antigen-binding fragment or other polypeptide specifically binds to an intracellular protein.

In certain embodiments, the antibody or antigen-binding fragment thereof or other polypeptide specifically binds to a cancer-associated antigen, or cancer antigen. Exemplary cancer antigens include cell surface proteins such as cell surface receptors. Also included as cancer-associated antigens are ligands that bind to such cell surface proteins or receptors. In specific embodiments, the antibody or antigen-binding fragment specifically binds to a intracellular cancer antigen. In some embodiments, the cancer that associates with the cancer antigen is one or more of breast cancer, metastatic brain cancer, prostate cancer, gastrointestinal cancer, lung cancer, ovarian cancer, testicular cancer, head and neck cancer, stomach cancer, bladder cancer, pancreatic cancer, liver cancer, kidney cancer, squamous cell carcinoma, CNS or brain cancer, melanoma, non-melanoma cancer, thyroid cancer, endometrial cancer, epithelial tumor, bone cancer, or a hematopoietic cancer.

In particular embodiments, the antibody or antigen-binding fragment or other polypeptide specifically binds to at least one cancer-associated antigen, or cancer antigen, such as human Her2/neu, HerI/EGF receptor (EGFR), Her3, A33 antigen, B7H3, CDS, CD19, CD20, CD22, CD23 (IgE Receptor), C242 antigen, ST4, IL-1, IL-la, IL-1p, IL-6, IL-13, vascular endothelial growth factor VEGF (e.g., VEGF-A) VEGFR-1, VEGFR-2, CD30, CD33, CD37, CD40, CD44, CDSI, CD52, CD56, CD74, CD80, CD152, CD200, CD221, CCR4, HLA-DR, CTLA-4, NPC-IC, tenascin, vimentin, insulin-like growth factor 1 receptor (IGF-IR), alpha-fetoprotein, insulin-like growth factor 1 (IGF-1), carbonic anhydrase 9 (CA-IX), carcinoembryonic antigen (CEA), integrin av 3, integrin a5 1, folate receptor 1, transmembrane glycoprotein NMB, fibroblast activation protein alpha (FAP), glycoprotein 75, TAG-72, MUCI, MUC16 (or CA-125), phosphatidylserine, prostate-specific membrane antigen (PMSA), NR-LU-13 antigen, TRAIL-RI, tumor necrosis factor receptor superfamily member 10b (TNFRSFIOB or TRAIL-R2), SLAM family member 7 (SLAMF7), EGP40 pancarcinoma antigen, B-cell activating factor (BAFF), platelet-derived growth factor receptor, glycoprotein EpCAM (17-IA), Programmed Death-1, protein disulfide isomerase (POI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and/or mesothelin.

In specific embodiments, the antibody or antigen-binding fragment thereof or other polypeptide specifically binds to the human Her2/neu protein. Essentially any anti-Her2/neu antibody, antigen-binding fragment or other Her2/neu-specific binding agent may be used in producing the p97-antibody conjugates of the present invention. Illustrative anti-Her2/neu antibodies are described, for example, in U.S. Pat. Nos. 5,677,171; 5,720,937; 5,720,954; 5,725,856; 5,770,195; 5,772,997; 6,165,464; 6,387,371; and 6,399,063, the contents of which are incorporated herein by reference in their entireties.

In some embodiments, the antibody or antigen-binding fragment thereof or other polypeptide specifically binds to the human HerI/EGFR (epidermal growth factor receptor). Essentially any anti-HerI/EGFR antibody, antigen-binding fragment or other HerI-EGFR-specific binding agent may be used in producing the p97-antibody conjugates of the present invention. Illustrative anti-HerI/EGFR antibodies are described, for example, in U.S. Pat. Nos. 5,844,093; 7,132,511; 7,247,301; 7,595,378; 7,723,484; 7,939,072; and 7,960,516, the contents of which are incorporated by reference in their entireties.

In certain embodiments, the antibody is a therapeutic antibody, such as an anti-cancer therapeutic antibody, including antibodies such as 3F8, 8H9, abagovomab, adecatumumab, afutuzumab, alemtuzumab, alacizumab (pegol), amatuximab, apolizumab, bavituximab, bectumomab, belimumab, bevacizumab, bivatuzumab (mertansine), brentuximab vedotin, canakinumab, cantuzumab (mertansine), cantuzumab (ravtansine), capromab (pendetide), catumaxomab, cetuximab, citatuzumab (bogatox), cixutumumab, clivatuzumab (tetraxetan), conatumumab, dacetuzumab, dalotuzumab, detumomab, drozitumab, ecromeximab, edrecolomab, elotuzumab, enavatuzumab, ensituximab, epratuzumab, ertumaxomab, etaracizumab, farletuzumab, FBTA05, figitumumab, flanvotumab, galiximab, gemtuzumab, ganitumab, gemtuzumab (ozogamicin), girentuximab, glembatumumab (vedotin), ibritumomab tiuxetan, icrucumab, igovomab, indatuximab ravtansine, intetumumab, inotuzumab ozogamicin, ipilimumab (MDX-101), iratumumab, labetuzumab, lexatumumab, lintuzumab, lorvotuzumab (mertansine), lucatumumab, lumiliximab, mapatumumab, matuzumab, milatuzumab, mitumomab, mogamulizumab, moxetumomab (pasudotox), nacolomab (tafenatox), naptumomab (estafenatox), narnatumab, necitumumab, nimotuzumab, nivolumab, Neuradiab® (with or without radioactive iodine), NR-LU-10, ofatumumab, olaratumab, onartuzumab, oportuzumab (monatox), oregovomab, panitumumab, patritumab, pemtumomab, pertuzumab, pritumumab, racotumomab, radretumab, ramucirumab, rilotumumab, rituximab, robatumumab, samalizumab, sibrotuzumab, siltuximab, tabalumab, taplitumomab (paptox), tenatumomab, teprotumumab, TGN1412, ticilimumab, tremelimumab, tigatuzumab, TNX-650, tositumomab, TRBS07, trastuzumab, tucotuzumab (celmoleukin), ublituximab, urelumab, veltuzumab, volociximab, votumumab, and zalutumumab. Also included are fragments, variants, and derivatives of these antibodies.

In particular embodiments, the antibody is a cardiotoxic antibody, that is, an antibody that displays cardiotoxicity when administered in an unconjugated form. Specific examples of antibodies that display cardiotoxicity include trastuzumab and bevacizumab.

In specific embodiments, the anti-Her2/neu antibody used in a p97 conjugate is trastuzumab (Herceptin®), or a fragment, variant or derivative thereof. Herceptin® is a Her2/neu-specific monoclonal antibody approved for the treatment of human breast cancer. In certain embodiments, a Her2/neu-binding antigen-binding fragment comprises one or more of the CDRs of a Her2/neu antibody. In this regard, it has been shown in some cases that the transfer of only the VHCDR3 of an antibody can be performed while still retaining desired specific binding (Barbas et al., PNAS. 92: 2529-2533, 1995). See also, Mclane et al., PNAS USA. 92:5214-5218, 1995; and Barbas et al., J. Am. Chem. Soc. 116:2161-2162, 1994.

In other specific embodiments, the anti-HerI/EGFR antibody used in a conjugate of the invention is cetuximab (Erbitux®), or a fragment or derivative thereof. In certain embodiments, an anti-HerI/EGFR binding fragment comprises one or more of the CDRs of a HerI/EGFR antibody such as cetuximab. Cetuximab is approved for the treatment of head and neck cancer, and colorectal cancer. Cetuximab is composed of the Fv (variable; antigen-binding) regions of the 225 murine EGFR monoclonal antibody specific for the N-terminal portion of human EGFR with human IgGI heavy and kappa light chain constant (framework) regions.

In some embodiments, the antibody or antigen-binding fragment or other polypeptide specifically binds to an antigen associated with (e.g., treatment of) at least one nervous system disorder, including disorders of the peripheral and/or central nervous system (CNS) disorder. In certain embodiments, the antibody or antigen-binding fragment or other polypeptide specifically binds to an antigen associated with (e.g., treatment of) pain, including acute pain, chronic pain, and neuropathic pain. In some embodiments, the antibody or antigen-binding fragment or other polypeptide specifically binds an antigen associated with (e.g., treatment of) an autoimmune disorder, including autoimmune disorders of the nervous system or CNS.

Examples of nervous system-, pain-, and/or autoimmune-associated antigens include, without limitation, alpha-4 (a4) integrin, CD20, CD52, IL-1, IL-1α, IL-1β, IL-12, IL-23, the p40 subunit of IL-12 and IL-23, and the axonal regrowth and remyelination inhibitors Noga-A and LINGO, IL-23, amyloid-β (e.g., Aβ₍₁₋₄₂₎), Huntingtin, CD25 (i.e., the alpha chain of the IL-2 receptor), nerve growth factor (NGF), neurotrophic tyrosine kinase receptor type 1 (TrkA; the high affinity catalytic receptor for NGF), and alpha-synuclein. These and other targets have been considered useful in the treatment of a variety of nervous system, pain, and/or autoimmune disorders, such as multiple sclerosis (a4 integrin, IL-23, CD25, CD20, CD52, IL-12, IL-23, the p40 subunit of IL-12 and IL-23, and the axonal regrowth and remyelination inhibitors Noga-A and LINGO), Alzheimer's Disease (A), Huntington's Disease (Huntingtin), Parkinson's Disease (alpha-synuclein), and pain (NGF and TrkA).

In specific embodiments, the anti-CD25 antibody used in a p97 conjugate is daclizumab (i.e., Zenapax™), or a fragment, variant or derivative thereof. Daclizumab a humanized monoclonal antibody that specifically binds to CD25, the alpha subunit of the IL-2 receptor. In other embodiments, the antibody is rituximab, ocrelizumab, ofatumumab, or a variant or fragment thereof that specifically binds to CD20. In particular embodiments, the antibody is alemtuzumab, or a variant or fragment thereof that specifically binds to CD52. In certain embodiments, the antibody is ustekinumab (CNTO 1275), or a variant or fragment thereof that specifically binds to the p40 subunit of IL-12 and IL-23.

In specific embodiments, the anti-NGF antibody used in a conjugate is tanezumab, or a fragment, variant or derivative thereof. Tanezumab specifically binds to NGF and prevents NGF from binding to its high affinity, membrane-bound, catalytic receptor tropomyosin-related kinase A (TrkA), which is present on sympathetic and sensory neurons; reduced stimulation of TrkA by NGF is believed to inhibit the pain-transmission activities of such neurons.

In some embodiments, the antibody or antigen-binding fragment thereof or other polypeptide (e.g., immunoglobulin-like molecule, soluble receptor, ligand) specifically binds to a pro-inflammatory molecule, for example, a pro-inflammatory cytokine or chemokine. In these and related embodiments, the p97 conjugate can be used to treat a variety of inflammatory conditions, as described herein.

Examples of pro-inflammatory molecules include tumor necrosis factors (TNF) such as TNF-a and TNF-, TNF superfamily molecules such as FasL, CD27L, CD30L, CD40L, Ox40L, 4-IBBL, TRAIL, TWEAK, and Apo3L, interleukin-1 (IL-1) including IL-1, IL-1α, IL-1β, IL-2, interferon-y (IFN-y), IFN-a/, IL-6, IL-8, IL-12, IL-15, IL-17, IL-18, IL-21, LIF, CCL5, GROa, MCP-1, MIP-la, MIP-1, macrophage colony stimulating factor (MCSF), granulocyte macrophage colony stimulating factor (GM-CSF), CXCL2, CCL2, among others. In some embodiments, the antibody or antigen-binding fragment thereof specifically binds to a receptor of one or more of the foregoing pro-inflammatory molecules, such as TNF receptor (TNFR), an IL-1 receptor (IL-1R), or an IL-6 receptor (IL-6R), among others.

In specific embodiments, as note above, the antibody or antigen-binding fragment or other polypeptide specifically binds to TNF-a or TNF- . In particular embodiments, the anti-TNF antibody or other TNF-binding polypeptide is adalimumab (Humira®), certolizumab pegol (Cimzia®), etanercept (Enbrel®), golimumab (Cimzia®), or infliximab (Remicade®), D2E7, CDP 571, or CDP 870, or an antigen-binding fragment or variant thereof. In some embodiments, the TNF-binding polypeptide is a soluble receptor or ligand, such as TNRFSFIOB, TRAIL (i.e., CD253), TNFSFIO, TRADD (tumor necrosis factor receptor type 1-associated DEATH domain protein), TRAFs (TNF receptor associated factors, including TRAFS 1-7), or RIP (ribosome-inactivating proteins). Conjugates comprising an anti-TNF antibody or TNF-binding polypeptide can be used, for instance, in the treatment of various inflammatory conditions, as described herein. Such p97 conjugates can also be used in the treatment of various neurological conditions or disorders such as Alzheimer's disease, stroke, traumatic brain injury (TBI), spinal stenosis, acute spinal cord injury, and spinal cord compression (see U.S. Pat. Nos. 6,015,557; 6,177,077; 6,419,934; 6,419,944; 6,537,549; 6,982,089; and 7,214,658).

In specific embodiments, as note above, the antibody or antigen-binding fragment specifically binds to IL-1, IL-1 alpha or IL-1 beta. In particular embodiments, the anti-IL-1 antibody is canakinumab or gevokizumab, or a variant or fragment thereof that specifically binds to IL-1. Among other inflammatory conditions described herein, p97 conjugates comprising an anti-IL-1 antibody can be used to treat cryopyrin-associated periodic syndromes (CAPS), including familial cold autoinflammatory syndrome, Muckle-Wells syndrome, and neonatal-onset multisystem inflammatory disease.

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995.

Particular examples include the VELOCIMMUNE® platform by REGENEREX® (see, e.g., U.S. Pat. No. 6,596,541).

Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2.

Other libraries suitable for use will be apparent to persons skilled in the art. The p97 polypeptides described herein and known in the art may be used in the purification process in, for example, an affinity chromatography step.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDRI,” “CDR2,” and “CDR3” respectively. An antigen-binding site, therefore, includes six CDRs, comprising the CDR set from each of a heavy and a light chain V region. A polypeptide comprising a single CDR, (e.g., a CDRI, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′h, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′h fragment which comprises both antigen-binding sites. An Fv fragment for use according to certain embodiments of the present invention can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. See Inbar et al., PNAS USA. 69:2659-2662, 1972; Hochman et al., Biochem. 15:2706-2710, 1976; and Ehrlich et al., Biochem. 19:4091-4096, 1980.

In certain embodiments, single chain Fv or scFV antibodies are contemplated. For example, Kappa bodies (Ill et al., Prat. Eng. 10:949-57, 1997); minibodies (Martin et al., EMBO J 13:5305-9, 1994); diabodies (Holliger et al., PNAS 90: 6444-8, 1993); or Janusins (Traunecker et al., EMBO J 10: 3655-59, 1991; and Traunecker et al., Int. J. Cancer Suppl. 7:51-52, 1992), may be prepared using standard molecular biology techniques following the teachings of the present application with regard to selecting antibodies having the desired specificity.

A single chain Fv (sFv) polypeptide is a covalently linked VH::VL heterodimer which is expressed from a gene fusion including Vw and VL-encoding genes linked by a peptide-encoding linker. Huston et al. (PNAS USA. 85(16):5879-5883, 1988). A number of methods have been described to discern chemical structures for converting the naturally aggregated-but chemically separated-light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a “diabody.”

Dia bodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). A dAb fragment of an antibody consists of a VH domain (Ward et al., Nature 341:544-546, 1989). Dia bodies and other multivalent or multispecific fragments can be constructed, for example, by gene fusion (see WO94/13804; and Holliger et al., PNAS USA. 90:6444-6448, 1993)).

Minibodies comprising a scFv joined to a CH3 domain are also included (see Hu et al., Cancer Res. 56:3055-3061, 1996). See also Ward et al., Nature. 341:544-546, 1989; Bird et al., Science. 242:423-426, 1988; Huston et al., PNAS USA. 85:5879-5883, 1988); PCT/US92/09965; WO94/13804; and Reiter et al., Nature Biotech. 14:1239-1245, 1996.

Where bispecific antibodies are to be used, these may be conventional bispecific antibodies, which can be manufactured in a variety of ways (Holliger and Winter, Current Opinion Biotechnol. 4:446-449, 1993), e.g. prepared chemically or from hybrid hybridomas, or may be any of the bispecific antibody fragments mentioned above. Dia bodies and scFv can be constructed without an Fe region, using only variable domains, potentially reducing the effects of anti-idiotypic reaction.

Bispecific diabodies, as opposed to bispecific whole antibodies, may also be particularly useful because they can be readily constructed and expressed inf. coli. Dia bodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (Ridgeway et al., Protein Eng., 9:616-621, 1996).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies provided herein may take the form of a nanobody. Minibodies are encoded by single genes and are efficiently produced in almost all prokaryotic and eukaryotic hosts, for example, E. coli (see U.S. Pat. No. 6,765,087), moulds (for example Aspergillus or Trichoderma) and yeast (for example Saccharomyces, Kluyvermyces, Hansenula or Pichia (see U.S. Pat. No. 6,838,254). The production process is scalable and multi-kilogram quantities of nanobodies have been produced. Nanobodies may be formulated as a ready-to-use solution having a long shelf life. The Nanoclone method (see WO 06/079372) is a proprietary method for generating Nanobodies against a desired target, based on automated high-throughput selection of B-cells.

In certain embodiments, the antibodies or antigen-binding fragments thereof are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988).

Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeyen et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present invention may be chimeric antibodies. In this regard, a chimeric antibody is comprised of an antigen-binding fragment of an antibody operably linked or otherwise fused to a heterologous Fe portion of a different antibody. In certain embodiments, the heterologous Fe domain is of human origin. In other embodiments, the heterologous Fe domain may be from a different Ig class from the parent antibody, including IgA (including subclasses IgAI and IgA2), IgD, IgE, IgG (including subclasses IgGI, IgG2, IgG3, and IgG4), and IgM. In further embodiments, the heterologous Fe domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

Peptide Mimetics. Certain embodiments employ “peptide mimetics.” Peptide analogs are commonly used in the pharmaceutical industry as non-peptide drugs with properties analogous to those of the template peptide. These types of non-peptide compound are termed “peptide mimetics” or “peptidomimetics” (Luthman et al., A Textbook of Drug Design and Development, 14:386-406, 2nd Ed., Harwood Academic Publishers, 1996; Joachim Grante, Angew. Chem. Int. Ed. Engl., 33:1699-1720, 1994; Fauchere, Adv. Drug Res., 15:29, 1986; Veber and Freidinger TINS, p. 392 (1985); and Evans et al., J. Med. Chem. 30:229, 1987). A peptidomimetic is a molecule that mimics the biological activity of a peptide but is no longer peptidic in chemical nature. Peptidomimetic compounds are known in the art and are described, for example, in U.S. Pat. No. 6,245,886.

A peptide mimetic can have the “specific binding” characteristics described for antibodies (supra). For example, a peptide mimetic can specifically bind to a target described herein with a binding affinity (Kd) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In some embodiments a peptide mimetic specifically binds to a cell surface receptor or other cell surface protein. In some embodiments, the peptide mimetic specifically binds to at least one cancer-associated antigen described herein. In particular embodiments, the peptide mimetic specifically binds to at least one nervous system-associated, pain-associated, and/or autoimmune-associated antigen described herein.

Peptoids. The conjugates of the present invention also includes “peptoids.” Peptoid derivatives of peptides represent another form of modified peptides that retain the important structural determinants for biological activity, yet eliminate the peptide bonds, thereby conferring resistance to proteolysis (Simon, et al., PNAS USA. 89:9367-9371, 1992). Peptoids are oligomers of N-substituted glycines. A number of N-alkyl groups have been described, each corresponding to the side chain of a natural amino acid. The peptidomimetics of the present invention include compounds in which at least one amino acid, a few amino acids or all amino acid residues are replaced by the corresponding N-substituted glycines. Peptoid libraries are described, for example, in U.S. Pat. No. 5,811,387.

A peptoid can have the “specific binding” characteristics described for antibodies (supra). For instance, a peptoid can specifically bind to a target described herein with a binding affinity (Kd) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In certain embodiments a peptoid specifically binds to a cell surface receptor or other cell surface protein. In some embodiments, the peptoid specifically binds to at least one cancer-associated antigen described herein. In particular embodiments, the peptoid specifically binds to at least one nervous system-associated, pain-associated, and/or autoimmune-associated antigen described herein.

Aptamers. The p97 conjugates of the present invention also include aptamers (see, e.g., Ellington et al., Nature. 346, 818-22, 1990; and Tuerk et al., Science. 249, 505-10, 1990). Examples of aptamers include nucleic acid aptamers (e.g., DNA aptamers, RNA aptamers) and peptide aptamers. Nucleic acid aptamers refer generally to nucleic acid species that have been engineered through repeated rounds of in vitro selection or equivalent method, such as SELEX (systematic evolution of ligands by exponential enrichment), to bind to various molecular targets such as small molecules, proteins, nucleic acids, and even cells, tissues and organisms. See, e.g., U.S. Pat. Nos. 6,376,190; and 6,387,620.

Peptide aptamers typically include a variable peptide loop attached at both ends to a protein scaffold, a double structural constraint that typically increases the binding affinity of the peptide aptamer to levels comparable to that of an antibody's (e.g., in the nanomolar range). In certain embodiments, the variable loop length may be composed of about 10-20 amino acids (including all integers in between), and the scaffold may include any protein that has good solubility and compacity properties. Certain exemplary embodiments may utilize the bacterial protein Thioredoxin-A as a scaffold protein, the variable loop being inserted within the reducing active site (-Cys-Gly-Pro-Cys- loop in the wild protein), with the two cysteines lateral chains being able to form a disulfide bridge. Methods for identifying peptide aptamers are described, for example, in U.S. Application No. 2003/0108532.

An aptamer can have the “specific binding” characteristics described for antibodies (supra). For instance, an aptamer can specifically bind to a target described herein with a binding affinity (Kd) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In particular embodiments, an aptamer specifically binds to a cell surface receptor or other cell surface protein. In some embodiments, the aptamer specifically binds to at least one cancer-associated antigen described herein. In particular embodiments, the aptamer specifically binds to at least one nervous system-associated, pain-associated, and/or autoimmune-associated antigen described herein.

Detectable Entities. In some embodiments, the p97 fragment is conjugated to a “detectable entity.” Exemplary detectable entities include, without limitation, iodine-based labels, radioisotopes, fluorophores/fluorescent dyes, and nanoparticles.

Exemplary iodine-based labels include diatrizoic acid (Hypaque®, GE Healthcare) and its anionic form, diatrizoate. Diatrizoic acid is a radio-contrast agent used in advanced X-ray techniques such as CT scanning. Also included are iodine radioisotopes, described below.

Exemplary radioisotopes that can be used as detectable entities include ³²P, ³³P, ³⁵S, ³H, ¹⁸F, ¹¹C, ¹³N, ¹⁵O, ¹¹¹N, ¹⁶⁹Yb, ^{99m}TC, ⁵⁵Fe and isotopes of iodine such as ¹²³I, ¹²⁴I, ¹²⁵I, and ¹³¹I. These radioisotopes have different half-lives, types of decay, and levels of energy which can be tailored to match the needs of a particular protocol. Certain of these radioisotopes can be selectively targeted or better targeted to CNS tissues by conjugation to p97 polypeptides, for instance, to improve the medical imaging of such tissues.

Examples of fluorophores or fluorochromes that can be used as directly detectable entities include fluorescein, tetramethylrhodamine, Texas Red, Oregon Green®, and a number of others (e.g., Haugland, Handbook of Fluorescent Probes—9th Ed., 2002, Malec. Probes, Inc., Eugene Oreg.; Haugland, The Handbook: A Guide to Fluorescent Probes and Labeling Technologies-10th Ed., 2005, Invitrogen, Carlsbad, Calif.). Also included are light-emitting or otherwise detectable dyes. The light emitted by the dyes can be visible light or invisible light, such as ultraviolet or infrared light. In exemplary embodiments, the dye may be a fluorescence resonance energy transfer (FRET) dye; a xanthene dye, such as fluorescein and rhodamine; a dye that has an amino group in the alpha or beta position (such as a naphthylamine dye, 1-dimethylaminonaphthyl-5-sulfonate, 1-anilino-8-naphthalende sulfonate and 2-p-touidinyl-6-naphthalene sulfonate); a dye that has 3-phenyl-7-isocyanatocoumarin; an acridine, such as 9-isothiocyanatoacridine and acridine orange; a pyrene, a bensoxadiazole and a stilbene; a dye that has 3-(s-carboxypentyl)-3′-ethyl-5,5′-dimethyloxacarbocyanine (CYA); 6-carboxy fluorescein (FAM); 5&6-carboxyrhodamine-110 (R110); 6-carboxyrhodamine-6G (R6G); N,N,N′,N′-tetramethyl-6-carboxyrhodamine (TAMRA); 6-carboxy-X-rhodamine (ROX); 6-carboxy-4′,5′-dichloro-2′,7′-dimethoxyfluorescein (JOE); ALEXA FLUOR™; Cy2; Texas Red and Rhodamine Red; 6-carboxy-2′,4,7,7′-tetrachlorofluorescein (TET); 6-carboxy-2′,4,4′,5′,7,7′-hexachlorofluorescein (HEX); 5-carboxy-2′,4′,5′,7′-tetrachlorofluorescein (ZOE); NAN; NED; Cy3; Cy3.5; Cy5; Cy5.5; Cy7; and Cy7.5; IR800CW, ICG, Alexa Fluor 350; Alexa Fluor 488; Alexa Fluor 532; Alexa Fluor 546; Alexa Fluor 568; Alexa Fluor 594; Alexa Fluor 647; Alexa Fluor 680, or Alexa Fluor 750. Certain embodiments include conjugation to chemotherapeutic agents (e.g., paclitaxel, adriamycin) that are labeled with a detectable entity, such as a fluorophore (e.g., Oregon Green®, Alexa Fluor 488).

Nanoparticles usually range from about 1-1000 nm in size and include diverse chemical structures such as gold and silver particles and quantum dots. When irradiated with angled incident white light, silver or gold nanoparticles ranging from about 40-120 nm will scatter monochromatic light with high intensity. The wavelength of the scattered light is dependent on the size of the particle. Four to five different particles in close proximity will each scatter monochromatic light, which when superimposed will give a specific, unique color. Derivatized nanoparticles such as silver or gold particles can be attached to a broad array of molecules including, proteins, antibodies, small molecules, receptor ligands, and nucleic acids. Specific examples of nanoparticles include metallic nanoparticles and metallic nanoshells such as gold particles, silver particles, copper particles, platinum particles, cadmium particles, composite particles, gold hollow spheres, gold-coated silica nanoshells, and silica-coated gold shells. Also included are silica, latex, polystyrene, polycarbonate, polyacrylate, PVDF nanoparticles, and colored particles of any of these materials.

Quantum dots are fluorescing crystals about 1-5 nm in diameter that are excitable by light over a large range of wavelengths. Upon excitation by light having an appropriate wavelength, these crystals emit light, such as monochromatic light, with a wavelength dependent on their chemical composition and size. Quantum dots such as CdSe, ZnSe, InP, or InAs possess unique optical properties; these and similar quantum dots are available from a number of commercial sources (e.g., NN-Labs, Fayetteville, Ark.; Ocean Nanotech, Fayetteville, Ark.; Nanoco Technologies, Manchester, UK; Sigma-Aldrich, St. Louis, Mo.).

Polypeptide Variants and Fragments. Certain embodiments include variants and/or fragments of the reference polypeptides described herein, whether described by name or by reference to a sequence identifier, including p97 polypeptides and polypeptide-based agents such as antibodies. The wild-type or most prevalent sequences of these polypeptides are known in the art, and can be used as a comparison for the variants and fragments described herein.

A polypeptide “variant,” as the term is used herein, is a polypeptide that typically differs from a polypeptide specifically disclosed herein by one or more substitutions, deletions, additions and/or insertions. Variant polypeptides are biologically active, that is, they continue to possess the enzymatic or binding activity of a reference polypeptide. Such variants may result from, for example, genetic polymorphism and/or from human manipulation.

In many instances, a biologically active variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant or portion of a polypeptide of the invention, one skilled in the art will typically change one or more of the codons of the encoding DNA sequence according to Table A below.

TABLE A

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their utility.

In making such changes, the hydropathic index of amino acids may be considered. The importance of the hydropathic amino acid index in conferring interactive biologic function on a protein is generally understood in the art (Kyte & Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte & Doolittle, 1982). These values are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); praline (−1.6); histidine (−3.2); glutamate (−3.5); glutamine (−3.5); aspartate (−3.5); asparagine (−3.5); lysine (−3.9); and arginine (−4.5). It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); praline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his.

A variant may also, or alternatively, contain non-conservative changes. In a preferred embodiment, variant polypeptides differ from a native sequence by substitution, deletion or addition of fewer than about 10, 9, 8, 7, 6, 5, 4, 3, 2 amino acids, or even 1 amino acid. Variants may also (or alternatively) be modified by, for example, the deletion or addition of amino acids that have minimal influence on the immunogenicity, secondary structure, enzymatic activity, and/or hydropathic nature of the polypeptide.

In certain embodiments, variants of the DSSHAFTLDELR (SEQ ID NO:2) can be based on the sequence of p97 sequences from other organisms, as shown in Table B of U.S. Pat. No. 9,364,567, issued Jun. 14, 2016, the entire contents of such patent is hereby incorporated by reference as if set out in full.

In general, variants will display at least about 30%, 40%, 50%, 55%, 60%, 65%, 70%, 75%, 80%, 85%, 90%, 91%, 92%, 93%, 94%, 95%, 96%, 97%, 98%, 99% similarity or sequence identity or sequence homology to a reference polypeptide sequence. Moreover, sequences differing from the native or parent sequences by the addition (e.g., (-terminal addition, N-terminal addition, both), deletion, truncation, insertion, or substitution of about 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties or activities of a parent or reference polypeptide sequence are contemplated.

In some embodiments, variant polypeptides differ from reference sequence by at least one but by less than 50, 40, 30, 20, 15, 10, 8, 6, 5, 4, 3 or 2 amino acid residue(s). In other embodiments, variant polypeptides differ from a reference sequence by at least 1% but less than 20%, 15%, 10% or 5% of the residues. (If this comparison requires alignment, the sequences should be aligned for maximum similarity. “Looped” out sequences from deletions or insertions, or mismatches, are considered differences.)

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (J. Mo/. Biol. 48: 444-453, 1970) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (Cabios. 4:11-17, 1989) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (Nucleic Acids Res. 25: 3389-3402, 1997). When utilizing BLAST and Gapped BLAST programs, the default parameters of the respective programs (e.g., XBLAST and NBLAST) can be used.

In one embodiment, as noted above, polynucleotides and/or polypeptides can be evaluated using a BLAST alignment tool. A local alignment consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone.

The raw score, S, is calculated from the number of gaps and substitutions associated with each aligned sequence wherein higher similarity scores indicate a more significant alignment. Substitution scores are given by a look-up table (see PAM, BLOSUM).

Gap scores are typically calculated as the sum of G, the gap opening penalty and L, the gap extension penalty. For a gap of length n, the gap cost would be G+Ln. The choice of gap costs, G and Lis empirical, but it is customary to choose a high value for G (10-15), e.g., 11, and a low value for L (1-2) e.g., 1.

The bit score, S′, is derived from the raw alignment score S in which the statistical properties of the scoring system used have been taken into account. Bit scores are normalized with respect to the scoring system, therefore they can be used to compare alignment scores from different searches. The terms “bit score” and “similarity score” are used interchangeably. The bit score gives an indication of how good the alignment is; the higher the score, the better the alignment.

The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e-117means that a sequence with a similar score is very unlikely to occur simply by chance. Additionally, the expected score for aligning a random pair of amino acids is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1.

In one embodiment, sequence similarity scores are reported from BLAST analyses done using the BLOSUM62 matrix, a gap existence penalty of 11 and a gap extension penalty of 1.

In a particular embodiment, sequence identity/similarity scores provided herein refer to the value obtained using GAP Version 10 (GCG, Accelrys, San Diego, Calif.) using the following parameters:% identity and % similarity for a nucleotide sequence using GAP Weight of 50 and Length Weight of 3, and the nwsgapdna.cmp scoring matrix;% identity and % similarity for an amino acid sequence using GAP Weight of 8 and Length Weight of 2, and the BLOSUM62 scoring matrix (Henikoff and Henikoff, PNAS USA. 89:10915-10919, 1992). GAP uses the algorithm of Needleman and Wunsch (J Mo/Biol. 48:443-453, 1970) to find the alignment of two complete sequences that maximizes the number of matches and minimizes the number of gaps.

As noted above, a reference polypeptide may be altered in various ways including amino acid substitutions, deletions, truncations, additions, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (PNAS USA. 82: 488-492, 1985); Kunkel et al., (Methods in Enzymol. 154: 367-382, 1987), U.S. Pat. No. 4,873,192, Watson, J. D. et al., (“Molecular Biology of the Gene,” Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by such modifications, and for screening cDNA libraries for gene products having a selected property are known in the art. Such methods are adaptable for rapid screening of the gene libraries generated by combinatorial mutagenesis of reference polypeptides. As one example, recursive ensemble mutagenesis (REM), a technique which enhances the frequency of functional mutants in the libraries, can be used in combination with the screening assays to identify polypeptide variants (Arkin and Yourvan, PNAS USA 89: 7811-7815, 1992; Delgrave et al., Protein Engineering. 6: 327-331, 1993).

Exemplary Methods for Conjugation. Conjugation or coupling of a p97 polypeptide sequence to an agent of interest can be carried out using standard chemical, biochemical and/or molecular techniques. Indeed, it will be apparent how to make a p97 conjugate in light of the present disclosure using available art-recognized methodologies. Of course, it will generally be preferred when coupling the primary components of a p97 conjugate of the present invention that the techniques employed and the resulting linking chemistries do not substantially disturb the desired functionality or activity of the individual components of the conjugate.

The particular coupling chemistry employed will depend upon the structure of the biologically active agent (e.g., small molecule, polypeptide), the potential presence of multiple functional groups within the biologically active agent, the need for protection/deprotection steps, chemical stability of the agent, and the like, and will be readily determined by one skilled in the art. Illustrative coupling chemistry useful for preparing the p97 conjugates of the invention can be found, for example, in Wong (1991), “Chemistry of Protein Conjugation and Crosslinking”, CRC Press, Boca Raton, Fla.; and Brinkley “A Brief Survey of Methods for Preparing Protein Conjugates with Dyes, Haptens, and Crosslinking Reagents,” in Bioconjug. Chem., 3:2013, 1992. Preferably, the binding ability and/or activity of the conjugate is not substantially reduced as a result of the conjugation technique employed, for example, relative to the unconjugated agent or the unconjugated p97 polypeptide.

In certain embodiments, a p97 polypeptide sequence may be coupled to an agent of interest either directly or indirectly. A direct reaction between a p97 polypeptide sequence and an agent of interest is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to indirectly couple a p97 polypeptide sequence and an agent of interest via a linker group, including non-peptide linkers and peptide linkers. A linker group can also function as a spacer to distance an agent of interest from the p97 polypeptide sequence in order to avoid interference with binding capabilities, targeting capabilities or other functionalities. A linker group can also serve to increase the chemical reactivity of a substituent on an agent, and thus increase the coupling efficiency. An increase in chemical reactivity may also facilitate the use of agents, or functional groups on agents, which otherwise would not be possible. The selection of releasable or stable linkers can also be employed to alter the pharmacokinetics of a p97 conjugate and attached agent of interest. Illustrative linking groups include, for example, disulfide groups, thioether groups, acid labile groups, photolabile groups, peptidase labile groups and esterase labile groups. In other illustrative embodiments, the conjugates include linking groups such as those disclosed in U.S. Pat. No. 5,208,020 or EP Patent O 425 235 BI, and Chari et al., Cancer Research. 52: 127-131, 1992. Additional exemplary linkers are described below.

In some embodiments, it may be desirable to couple more than one p97 polypeptide sequence to an agent, or vice versa. For example, in certain embodiments, multiple p97 polypeptide sequences are coupled to one agent, or alternatively, one or more p97 polypeptides are conjugated to multiple agents. The p97 polypeptide sequences can be the same or different. Regardless of the particular embodiment, conjugates containing multiple p97 polypeptide sequences may be prepared in a variety of ways. For example, more than one polypeptide may be coupled directly to an agent, or linkers that provide multiple sites for attachment can be used. Any of a variety of known heterobifunctional crosslinking strategies can be employed for making conjugates of the invention. It will be understood that many of these embodiments can be achieved by controlling the stoichiometries of the materials used during the conjugation/crosslinking procedure.

In certain exemplary embodiments, a reaction between an agent comprising a succinimidyl ester functional group and a p97 polypeptide comprising an amino group forms an amide linkage; a reaction between an agent comprising a oxycarbonylimidizaole functional group and a P97 polypeptide comprising an amino group forms an carbamate linkage; a reaction between an agent comprising a p-nitrophenyl carbonate functional group and a P97 polypeptide comprising an amino group forms an carbamate linkage; a reaction between an agent comprising a trichlorophenyl carbonate functional group and a P97 polypeptide comprising an amino group forms an carbamate linkage; a reaction between an agent comprising a thio ester functional group and a P97 polypeptide comprising an n-terminal amino group forms an amide linkage; a reaction between an agent comprising a proprionaldehyde functional group and a P97 polypeptide comprising an amino group forms a secondary amine linkage.

In some exemplary embodiments, a reaction between an agent comprising a butyraldehyde functional group and a P97 polypeptide comprising an amino group forms a secondary amine linkage; a reaction between an agent comprising an acetal functional group and a P97 polypeptide comprising an amino group forms a secondary amine linkage; a reaction between an agent comprising a piperidone functional group and a P97 polypeptide comprising an amino group forms a secondary amine linkage; a reaction between an agent comprising a methylketone functional group and a P97 polypeptide comprising an amino group forms a secondary amine linkage; a reaction between an agent comprising a tresylate functional group and a P97 polypeptide comprising an amino group forms a secondary amine linkage; a reaction between an agent comprising a maleimide functional group and a P97 polypeptide comprising an amino group forms a secondary amine linkage; a reaction between an agent comprising a aldehyde functional group and a P97 polypeptide comprising an amino group forms a secondary amine linkage; and a reaction between an agent comprising a hydrazine functional group and a P97 polypeptide comprising an carboxylic acid group forms a secondary amine linkage.

In particular exemplary embodiments, a reaction between an agent comprising a maleimide functional group and a P97 polypeptide comprising a thiol group forms a thio ether linkage; a reaction between an agent comprising a vinyl sulfone functional group and a P97 polypeptide comprising a thiol group forms a thio ether linkage; a reaction between an agent comprising a thiol functional group and a P97 polypeptide comprising a thiol group forms a di-sulfide linkage; a reaction between an agent comprising a orthopyridyl disulfide functional group and a P97 polypeptide comprising a thiol group forms a di-sulfide linkage; and a reaction between an agent comprising an iodoacetamide functional group and a P97 polypeptide comprising a thiol group forms a thio ether linkage.

In a specific embodiment, an amine-to-sulfhydryl crosslinker is used for preparing a conjugate.

In one preferred embodiment, for example, the crosslinker is succinimidyl-4-(N-maleimidomethyl)cyclohexane-1-carboxylate (SMCC) (Thermo Scientific), which is a sulfhydryl crosslinker containing NHS-ester and maleimide reactive groups at opposite ends of a medium-length cyclohexane-stabilized spacer arm (8.3 angstroms). SMCC is a non-cleavable and membrane permeable crosslinker that can be used to create sulfhydryl-reactive, maleimide-activated agents (e.g., polypeptides, antibodies) for subsequent reaction with p97 polypeptide sequences. NHS esters react with primary amines at pH 7-9 to form stable amide bonds. Maleimides react with sulfhydryl groups at pH 6.5-7.5 to form stable thioether bonds. Thus, the amine reactive NHS ester of SMCC crosslinks rapidly with primary amines of an agent and the resulting sulfhydryl-reactive maleimide group is then available to react with cysteine residues of p97 to yield specific conjugates of interest.

In certain specific embodiments, the p97 polypeptide sequence is modified to contain exposed sulfhydryl groups to facilitate crosslinking, e.g., to facilitate crosslinking to a maleimide-activated agent. In a more specific embodiment, the p97 polypeptide sequence is modified with a reagent which modifies primary amines to add protected thiol sulfhydryl groups. In an even more specific embodiment, the reagent N-succinimidyl-S-acetylthioacetate (SATA) (Thermo Scientific) is used to produce thiolated p97 polypeptides.

In other specific embodiments, a maleimide-activated agent is reacted under suitable conditions with thiolated p97 polypeptides to produce a conjugate of the present invention. It will be understood that by manipulating the ratios of SMCC, SATA, agent, and p97 polypeptide in these reactions it is possible to produce conjugates having differing stoichiometries, molecular weights and properties.

In still other illustrative embodiments, conjugates are made using bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particular coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage.

The specific crosslinking strategies discussed herein are but a few of many examples of suitable conjugation strategies that may be employed in producing conjugates of the invention. It will be evident to those skilled in the art that a variety of other bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Particular embodiments may employ one or more aldehyde tags to facilitate conjugation between a p97 polypeptide and an agent (see U.S. Pat. Nos. 8,097,701 and 7,985,783, incorporated by reference). Here, enzymatic modification at a sulfatase motif of the aldehyde tag through action of a formylglycine generating enzyme (FGE) generates a formylglycine (FGly) residue. The aldehyde moiety of the FGly residue can then be exploited as a chemical handle for site-specific attachment of a moiety of interest to the polypeptide. In some aspects, the moiety of interest is a small molecule, peptoid, aptamer, or peptide mimetic. In some aspects, the moiety of interest is another polypeptide, such as an antibody.

Polypeptides with the above-described motif can be modified by an FGE enzyme to generate a motif having a FGly residue, which, as noted above, can then be used for site-specific attachment of an agent, such as a second polypeptide, for instance, via a linker moiety. Such modifications can be performed, for example, by expressing the sulfatase motif-containing polypeptide (e.g., p97, antibody) in a mammalian, yeast, or bacterial cell that expresses an FGE enzyme or by in vitro modification of isolated polypeptide with an isolated FGE enzyme (see Wu et al., PNAS. 106:3000-3005, 2009; Rush and Bertozzi, J. Am Chem Soc. 130:12240-1, 2008; and Carlson et al., J Biol Chem. 283:20117-25, 2008).

The agent or non-aldehyde tag-containing polypeptide (e.g., antibody, p97 polypeptide) can be functionalized with one or more aldehyde reactive groups such as aminooxy, hydrazide, and thiosemicarbazide, and then covalently linked to the aldehyde tag-containing polypeptide via the at least one FGly residue, to form an aldehyde reactive linkage. The attachment of an aminooxy functionalized agent (or non-aldehyde tag-containing polypeptide) creates an oxime linkage between the FGly residue and the functionalized agent (or non-aldehyde tag-containing polypeptide); attachment of a hydrazide-functionalized agent (or non-aldehyde tag-containing polypeptide) creates a hydrazine linkage between the FGly residue and the functionalized agent (or non-aldehyde tag-containing polypeptide); and attachment of a thiosemicarbazide-functionalized agent (or non-aldehyde tag-containing polypeptide) creates a hydrazine carbothiamide linkage between the FGly residue and the functionalized agent (or non-aldehyde tag-containing polypeptide). Hence, in these and related embodiments, R1 can be a linkage that comprises a Schiff base, such as an oxime linkage, a hydrazine linkage, or a hydrazine carbothiamide linkage.

Certain embodiments include conjugates of (i) a sulfatase motif (or aldehyde tag)-containing p97 polypeptide and (ii) a sulfatase motif (or aldehyde tag)-containing polypeptide agent (A), where (i) and (ii) are covalently linked via their respective FGly residues, optionally via a bi-functionalized linker moiety or group.

In some embodiments, the aldehyde tag-containing p97 polypeptide and the aldehyde tag-containing agent are linked (e.g., covalently linked) via a multi-functionalized linker (e.g., bi-functionalized linker), the latter being functionalized with the same or different aldehyde reactive group(s). In these and related embodiments, the aldehyde reactive groups allow the linker to form a covalent bridge between the p97 polypeptide and the agent via their respective FGly residues. Linker moieties include any moiety or chemical that can be functionalized and preferably bi- or multi-functionalized with one or more aldehyde reactive groups. Particular examples include peptides, water-soluble polymers, detectable entities, other therapeutic compounds (e.g., cytotoxic compounds), biotin/streptavidin moieties, and glycans (see Hudak et al., J Am Chem Soc. 133:16127-35, 2011).

Specific examples of glycans (or glycosides) include aminooxy glycans, such as higher-order glycans composed of glycosyl N-pentenoyl hydroxamates intermediates (supra). Exemplary linkers are described herein, and can be functionalized with aldehyde reactive groups according to routine techniques in the art (see, e.g., Carrico et al., Nat Chem Biol. 3:321-322, 2007; and U.S. Pat. Nos. 8,097,701 and 7,985,783).

p97 conjugates can also be prepared by a various “click chemistry” techniques, including reactions that are modular, wide in scope, give very high yields, generate mainly inoffensive byproducts that can be removed by non-chromatographic methods, and can be stereospecific but not necessarily enantioselective (see Kolb et al., Angew Chem Int Ed Engl. 40:2004-2021, 2001). Particular examples include conjugation techniques that employ the Huisgen 1,3-dipolar cycloaddition of azides and alkynes, also referred to as “azide-alkyne cycloaddition” reactions (see Hein et al., Pharm Res. 25:2216-2230, 2008). Non-limiting examples of azide-alkyne cycloaddition reactions include copper-catalyzed azide-alkyne cycloaddition (CuAAC) reactions and ruthenium-catalyzed azide-alkyne cycloaddition (RuAAC) reactions.

CuAAC works over a broad temperature range, is insensitive to aqueous conditions and a pH range over 4 to 12, and tolerates a broad range of functional groups (see Himo et al, J Am Chem Soc. 127:210-216, 2005). The active Cu(I) catalyst can be generated, for example, from Cu(I) salts or Cu(II) salts using sodium ascorbate as the reducing agent. This reaction forms 1,4-substituted products, making it region-specific (see Hein et al., supra).

RuAAC utilizes pentamethylcyclopentadienyl ruthenium chloride [Cp*RuCl] complexes that are able to catalyze the cycloaddition of azides to terminal alkynes, regioselectively leading to 1,5-disubstituted 1,2,3-triazoles (see Rasmussen et al., Org. Lett. 9:5337-5339, 2007). Further, and in contrast to CuAAC, RuAAC can also be used with internal alkynes to provide fully substituted 1,2,3-triazoles.

Certain embodiments thus include p97 polypeptides that comprise at least one unnatural amino acid with an azide side-chain or an alkyne side-chain, including internal and terminal unnatural amino acids (e.g., N-terminal, (-terminal). Certain of these p97 polypeptides can be formed by in vivo or in vitro (e.g., cell-free systems) incorporation of unnatural amino acids that contain azide side-chains or alkyne side-chains. Exemplary in vivo techniques include cell culture techniques, for instance, using modified E. coli (see Travis and Schultz, The Journal of Biological Chemistry. 285:11039-44, 2010; and Deiters and Schultz, Bioorganic & Medicinal Chemistry Letters. 15:1521-1524, 2005), and exemplary in vitro techniques include cell-free systems (see Bundy, Bioconjug Chem. 21:255-63, 2010).

In some embodiments, a p97 polypeptide that comprises at least one unnatural amino acid with an azide side-chain is conjugated by azide-alkyne cycloaddition to an agent (or linker) that comprises at least one alkyne group, such as a polypeptide agent that comprises at least one unnatural amino acid with an alkyne side-chain. In other embodiments, a p97 polypeptide that comprises at least one unnatural amino acid with an alkyne side-chain is conjugated by azide-alkyne cycloaddition to an agent (or linker) that comprises at least one azide group, such as a polypeptide agent that comprises at least one unnatural amino acid with an azide side-chain. Hence, certain embodiments include conjugates that comprise a p97 polypeptide covalently linked to an agent via a 1,2,3-triazole linkage.

In certain embodiments, the unnatural amino acid with the azide side-chain and/or the unnatural amino acid with alkyne side-chain are terminal amino acids (N-terminal, (-terminal). In certain embodiments, one or more of the unnatural amino acids are internal.

For instance, certain embodiments include a p97 polypeptide that comprises an N-terminal unnatural amino acid with an azide side-chain conjugated to an agent that comprises an alkyne group. Some embodiments, include a p97 polypeptide that comprises a (-terminal unnatural amino acid with an azide side-chain conjugated to an agent that comprises an alkyne group. Particular embodiments include a p97 polypeptide that comprises an N-terminal unnatural amino acid with an alkyne side-chain conjugated to an agent that comprises an azide side-group. Further embodiments include a p97 polypeptide that comprises an (-terminal unnatural amino acid with an alkyne side-chain conjugated to an agent that comprises an azide side-group. Some embodiments include a p97 polypeptide that comprises at least one internal unnatural amino acid with an azide side-chain conjugated to an agent that comprises an alkyne group. Additional embodiments include a p97 polypeptide that comprises at least one internal unnatural amino acid with an alkyne side-chain conjugated to an agent that comprises an azide side-group.

Particular embodiments include a p97 polypeptide that comprises an N-terminal unnatural amino acid with an azide side-chain conjugated to a polypeptide agent that comprises an N-terminal unnatural amino acid with an alkyne side-chain. Other embodiments include a p97 polypeptide that comprises a (-terminal unnatural amino acid with an azide side-chain conjugated to a polypeptide agent that comprises a (-terminal unnatural amino acid with an alkyne side-chain. Still other embodiments include a p97 polypeptide that comprises an N-terminal unnatural amino acid with an azide side-chain conjugated to a polypeptide agent that comprises a (-terminal unnatural amino acid with an alkyne side-chain. Further embodiments include a p97 polypeptide that comprises a (-terminal unnatural amino acid with an azide side-chain conjugated to a polypeptide agent that comprises an N-terminal unnatural amino acid with an alkyne side-chain.

Other embodiments include a p97 polypeptide that comprises an N-terminal unnatural amino acid with an alkyne side-chain conjugated to a polypeptide agent that comprises an N-terminal unnatural amino acid with an azide side-chain. Still further embodiments include a p97 polypeptide that comprises a (-terminal unnatural amino acid with an alkyne side-chain conjugated to a polypeptide agent that comprises a (-terminal unnatural amino acid with an azide side-chain. Additional embodiments include a p97 polypeptide that comprises an N-terminal unnatural amino acid with an alkyne side-chain conjugated to a polypeptide agent that comprises a (-terminal unnatural amino acid with an azide side-chain. Still further embodiments include a p97 polypeptide that comprises a (-terminal unnatural amino acid with an alkyne side-chain conjugated to a polypeptide agent that comprises an N-terminal unnatural amino acid with an azide side-chain.

Also included are methods of producing a p97 conjugate, comprising: (a) performing an azide-alkyne cycloaddition reaction between (i) a p97 polypeptide that comprises at least one unnatural amino acid with an azide side-chain and an agent that comprises at least one alkyne group (for instance, an unnatural amino acid with an alkyne side chain); or (ii) a p97 polypeptide that comprises at least one unnatural amino acid with an alkyne side-chain and an agent that comprises at least one azide group (for instance, an unnatural amino acid with an azide side-chain); and (b) isolating a p97 conjugate from the reaction, thereby producing a p97 conjugate.

In the case where the p97 conjugate is a fusion polypeptide, the fusion polypeptide may generally be prepared using standard techniques. Preferably, however, a fusion polypeptide is expressed as a recombinant polypeptide in an expression system, described herein and known in the art. Fusion polypeptides of the invention can contain one or multiple copies of a p97 polypeptide sequence and may contain one or multiple copies of a polypeptide-based agent of interest (e.g., antibody or antigen-binding fragment thereof), present in any desired arrangement.

For fusion proteins, DNA sequences encoding the p97 polypeptide, the polypeptide agent (e.g., antibody), and optionally peptide linker components may be assembled separately, and then ligated into an appropriate expression vector. The 3′ end of the DNA sequence encoding one polypeptide component is ligated, with or without a peptide linker, to the 5′ end of a DNA sequence encoding the other polypeptide component(s) so that the reading frames of the sequences are in phase. The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the most (-terminal polypeptide. This permits translation into a single fusion polypeptide that retains the biological activity of both component polypeptides.

Similar techniques, mainly the arrangement of regulatory elements such as promoters, stop codons, and transcription termination signals, can be applied to the recombinant production of non-fusion proteins, for instance, p97 polypeptides and polypeptide agents (e.g., antibody agents) for the production of non-fusion conjugates.

Polynucleotides and fusion polynucleotides of the invention can contain one or multiple copies of a nucleic acid encoding a p97 polypeptide sequence, and/or may contain one or multiple copies of a nucleic acid encoding a polypeptide agent.

In some embodiments, a nucleic acids encoding a subject p97 polypeptide, polypeptide agent, and/or p97-polypeptide fusion are introduced directly into a host cell, and the cell incubated under conditions sufficient to induce expression of the encoded polypeptide(s). The polypeptide sequences of this disclosure may be prepared using standard techniques well known to those of skill in the art in combination with the polypeptide and nucleic acid sequences provided herein.

Therefore, according to certain related embodiments, there is provided a recombinant host cell which comprises a polynucleotide or a fusion polynucleotide that encodes a polypeptide described herein. Expression of a p97 polypeptide, polypeptide agent, or p97-polypeptide agent fusion in the host cell may conveniently be achieved by culturing under appropriate conditions recombinant host cells containing the polynucleotide. Following production by expression, the polypeptide(s) may be isolated and/or purified using any suitable technique, and then used as desired.

Systems for cloning and expression of a polypeptide in a variety of different host cells are well known. Suitable host cells include bacteria, mammalian cells, yeast and baculovirus systems.

Mammalian cell lines available in the art for expression of a heterologous polypeptide include Chinese hamster ovary (CHO) cells, Hela cells, baby hamster kidney cells, HEK-293 cells, NSO mouse melanoma cells and many others. A common, preferred bacterial host is f. coli. The expression of polypeptides in prokaryotic cells such as f. coli is well established in the art. For a review, see for example Pluckthun, A. Bio/Technology. 9:545-551 (1991). Expression in eukaryotic cells in culture is also available to those skilled in the art as an option for recombinant production of polypeptides (see Ref, Curr. Opinion Biotech. 4:573-576, 1993; and Trill et al., Curr. Opinion Biotech. 6:553-560, 1995.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the polypeptides described herein, and which further expresses or is capable of expressing a selected gene of interest, such as a gene encoding any herein described polypeptide. The term includes the progeny of the parent cell, whether or not the progeny are identical in morphology or in genetic make-up to the original parent, so long as the selected gene is present. Host cells may be chosen for certain characteristics, for instance, the expression of a formylglycine generating enzyme (FGE) to convert a cysteine or serine residue within a sulfatase motif into a formylglycine (FGly) residue, or the expression of aminoacyl tRNA synthetase(s) that can incorporate unnatural amino acids into the polypeptide, including unnatural amino acids with an azide side-chain, alkyne side-chain, or other desired side-chain, to facilitate conjugation.

Accordingly there is also contemplated a method comprising introducing such nucleic acid(s) into a host cell. The introduction of nucleic acids may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. In one embodiment, the nucleic acid is integrated into the genome (e.g. chromosome) of the host cell. Integration may be promoted by inclusion of sequences which promote recombination with the genome, in accordance-with standard techniques.

The present invention also provides, in certain embodiments, a method which comprises using a nucleic acid construct described herein in an expression system in order to express a particular polypeptide, such as a p97 polypeptide, polypeptide agent, or p97-polypeptide agent fusion protein as described herein.

As noted above, certain p97 conjugates, such as fusion proteins, may employ one or more linker groups, including non-peptide linkers (e.g., non-proteinaceous linkers) and peptide linkers. Such linkers can be stable linkers or releasable linkers.

Exemplary non-peptide stable linkages include succinimide, propionic acid, carboxymethylate linkages, ethers, carbamates, amides, amines, carbamides, imides, aliphatic C—C bonds, thio ether linkages, thiocarbamates, thiocarbamides, and the like. Generally, a hydrolytically stable linkage is one that exhibits a rate of hydrolysis of less than about 1-2% to 5% per day under physiological conditions.

Exemplary non-peptide releasable linkages include carboxylate ester, phosphate ester, anhydride, acetal, ketal, acyloxyalkyl ether, imine, orthoester, thio ester, thiol ester, carbonate, and hydrazone linkages. Other illustrative examples of releasable linkers can be benzyl elimination-based linkers, trialkyl lock-based linkers (or trialkyl lock lactonization based), bicine-based linkers, and acid labile linkers. Among the acid labile linkers can be disulfide bond, hydrazone-containing linkers and thiopropionate-containing linkers. Also included are linkers that are releasable or cleavable during or upon internalization into a cell. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.). In one embodiment, an acid-labile linker may be used (Cancer Research 52:127-131, 1992; and U.S. Pat. No. 5,208,020). Further details are known to those skilled in the art. See, For example, U.S. Pat. No. 9,364,567.

In certain embodiments, “water soluble polymers” are used in a linker for coupling a p97 polypeptide sequence to an agent of interest. A “water-soluble polymer” refers to a polymer that is soluble in water and is usually substantially non-immunogenic, and usually has an atomic molecular weight greater than about 1,000 Daltons. Attachment of two polypeptides via a water-soluble polymer can be desirable as such modification(s) can increase the therapeutic index by increasing serum half-life, for instance, by increasing proteolytic stability and/or decreasing renal clearance. Additionally, attachment via of one or more polymers can reduce the immunogenicity of protein pharmaceuticals.

Particular examples of water soluble polymers include polyethylene glycol, polypropylene glycol, polyoxyalkylenes, or copolymers of polyethylene glycol, polypropylene glycol, and the like.

In some embodiments, the water-soluble polymer has an effective hydrodynamic molecular weight of greater than about 10,000 Da, greater than about 20,000 to 500,000 Da, greater than about 40,000 Dato 300,000 Da, greater than about 50,000 Dato 70,000 Da, usually greater than about 60,000 Da. The “effective hydrodynamic molecular weight” refers to the effective water-solvated size of a polymer chain as determined by aqueous-based size exclusion chromatography (SEC). When the water-soluble polymer contains polymer chains having polyalkylene oxide repeat units, such as ethylene oxide repeat units, each chain can have an atomic molecular weight of between about 200 Da and about 80,000 Da, or between about 1,500 Da and about 42,000 Da, with 2,000 to about 20,000 Da being of particular interest. Linear, branched, and terminally charged water soluble polymers are also included.

Polymers useful as linkers between aldehyde tagged polypeptides can have a wide range of molecular weights, and polymer subunits. These subunits may include a biological polymer, a synthetic polymer, or a combination thereof. Examples of such water-soluble polymers include: dextran and dextran derivatives, including dextran sulfate, P-amino cross linked dextrin, and carboxymethyl dextrin, cellulose and cellulose derivatives, including methylcellulose and carboxymethyl cellulose, starch and dextrines, and derivatives and hydroylactes of starch, polyalklyene glycol and derivatives thereof, including polyethylene glycol (PEG), methoxypolyethylene glycol, polyethylene glycol homopolymers, polypropylene glycol homopolymers, copolymers of ethylene glycol with propylene glycol, wherein said homopolymers and copolymers are unsubstituted or substituted at one end with an alkyl group, heparin and fragments of heparin, polyvinyl alcohol and polyvinyl ethyl ethers, polyvinylpyrrolidone, aspartamide, and polyoxyethylated polyols, with the dextran and dextran derivatives, dextrine and dextrine derivatives. It will be appreciated that various derivatives of the specifically described water-soluble polymers are also included.

Water-soluble polymers are known in the art, particularly the polyalkylene oxide-based polymers such as polyethylene glycol “PEG” (see Poly(ethylene glycol) Chemistry: Biotechnical and Biomedical Applications, J. M. Harris, Ed., Plenum Press, New York, N.Y. (1992); and Poly(ethylene glycol) Chemistry and Biological Applications, J. M. Harris and S. Zalipsky, Eds., ACS (1997); and International Patent Applications: WO 90/13540, WO 92/00748, WO 92/16555, WO 94/04193, WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964, and U.S. Pat. Nos. 4,179,337; 5,075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001; 5,352,756; 5,405,877; 5,455,027; 5,446,090; 5,470,829; 5,478,805; 5,567,422; 5,605,976; 5,612,460; 5,614,549; 5,618,528; 5,672,662; 5,637,749; 5,643,575; 5,650,388; 5,681,567; 5,686,110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077,939; 6,113,906; 6,127,355; 6,177,087; 6,180,095; 6,194,580; 6,214,966, incorporated by reference).

Exemplary polymers of interest include those containing a polyalkylene oxide, polyamide alkylene oxide, or derivatives thereof, including polyalkylene oxide and polyamide alkylene oxide comprising an ethylene oxide repeat unit. Further exemplary polymers of interest include a polyamide having a molecular weight greater than about 1,000 Daltons. Further exemplary water-soluble repeat units comprise an ethylene oxide. The number of such water-soluble repeat units can vary significantly, with the usual number of such units being from 2 to 500, 2 to 400, 2 to 300, 2 to 200, 2 to 100, and most usually 2 to 50.

In certain embodiments, a peptide linker sequence may be employed to separate or couple the components of a p97 conjugate. For instance, for polypeptide-polypeptide conjugates, peptide linkers can separate the components by a distance sufficient to ensure that each polypeptide folds into its secondary and tertiary structures. Such a peptide linker sequence may be incorporated into the conjugate (e.g., fusion protein) using standard techniques described herein and well-known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180.

In certain illustrative embodiments, a peptide linker is between about 1 to 5 amino acids, between 5 to 10 amino acids, between 5 to 25 amino acids, between 5 to 50 amino acids, between 10 to 25 amino acids, between 10 to 50 amino acids, between 10 to 100 amino acids, or any intervening range of amino acids. In other illustrative embodiments, a peptide linker comprises about 1, 5, 10, 15, 20, 25, 30, 35, 40, 45, 50 or more amino acids in length. Particular linkers can have an overall amino acid length of about 1-200 amino acids, 1-150 amino acids, 1-100 amino acids, 1-90 amino acids, 1-80 amino acids, 1-70 amino acids, 1-60 amino acids, 1-50 amino acids, 1-40 amino acids, 1-30 amino acids, 1-20 amino acids, 1-10 amino acids, 1-5 amino acids, 1-4 amino acids, 1-3 amino acids, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more amino acids.

A peptide linker may employ any one or more naturally-occurring amino acids, non-naturally occurring amino acid(s), amino acid analogs, and/or amino acid mimetics as described elsewhere herein and known in the art. Certain amino acid sequences which may be usefully employed as linkers include those disclosed in Maratea et al., Gene 40:39-46, 1985; Murphy et al., PNAS USA. 83:8258-8262, 1986;

U.S. Pat. Nos. 4,935,233 and 4,751,180. Particular peptide linker sequences contain Gly, Ser, and/or Asn residues. Other near neutral amino acids, such as Thr and Ala may also be employed in the peptide linker sequence, if desired. Other combinations of these and related amino acids will be apparent to persons skilled in the art.

In specific embodiments, the linker sequence comprises a Gly3 linker sequence, which includes three glycine residues. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

The peptide linkers may be physiologically stable or may include a releasable linker such as a physiologically degradable or enzymatically degradable linker (e.g., proteolytically cleavable linker). In certain embodiments, one or more releasable linkers can result in a shorter half-life and more rapid clearance of the conjugate. These and related embodiments can be used, for example, to enhance the solubility and blood circulation lifetime of p97 conjugates in the bloodstream, while also delivering an agent into the bloodstream (or across the BBB) that, subsequent to linker degradation, is substantially free of the p97 sequence. These aspects are especially useful in those cases where polypeptides or other agents, when permanently conjugated to a p97 sequence, demonstrate reduced activity. By using the linkers as provided herein, such antibodies can maintain their therapeutic activity when in conjugated form. In these and other ways, the properties of the p97 conjugates can be more effectively tailored to balance the bioactivity and circulating half-life of the antibodies over time.

Enzymatically degradable linkages suitable for use in particular embodiments of the present invention include, but are not limited to: an amino acid sequence cleaved by a serine protease such as thrombin, chymotrypsin, trypsin, elastase, kallikrein, or substilisin.

Enzymatically degradable linkages suitable for use in particular embodiments of the present invention also include amino acid sequences that can be cleaved by a matrix metalloproteinase such as collagenase, stromelysin, and gelatinase.

Enzymatically degradable linkages suitable for use in particular embodiments of the present invention also include amino acid sequences that can be cleaved by an angiotensin converting enzyme.

Enzymatically degradable linkages suitable for use in particular embodiments of the present invention also include amino acid sequences that can be degraded by cathepsin B.

In certain embodiments, however, any one or more of the non-peptide or peptide linkers are optional. For instance, linker sequences may not required in a fusion protein where the first and second polypeptides have non-essential N-terminal and/or (-terminal amino acid regions that can be used to separate the functional domains and prevent steric interference.

The functional properties of the p97 polypeptides and p97 polypeptide conjugates described herein may be assessed using a variety of methods known to the skilled person, including, e.g., affinity/binding assays (for example, surface plasmon resonance, competitive inhibition assays); cytotoxicity assays, cell viability assays, cell proliferation or differentiation assays, cancer cell and/or tumor growth inhibition using in vitro or in vivo models. For instance, the conjugates described herein may be tested for effects on receptor internalization, in vitro and in vivo efficacy, etc., including the rate of transport across the blood brain barrier. Such assays may be performed using well-established protocols known to the skilled person (see e.g., Current Protocols in Molecular Biology (Greene Publ. Assoc. Inc. & John Wiley & Sons, Inc., NY, NY); Current Protocols in Immunology (Edited by: John E. Coligan, Ada M. Kruisbeek, David H. Margulies, Ethan M. Shevach, Warren Strober 2001John Wiley & Sons, NY, NY); or commercially available kits.

Methods of Use and Pharmaceutical Compositions

Certain embodiments of the present invention relate to methods of using the compositions of p97 polypeptides and p97 conjugates described herein. Examples of such methods include methods of treatment and methods of diagnosis, including for instance, the use of p97 conjugates for medical imaging of certain organs/tissues, such as those of the nervous system. Specific embodiments include methods of diagnosing and/or treating disorders or conditions of the central nervous system (CNS), or disorders or conditions having a CNS component.

Accordingly, certain embodiments include methods of treating a subject in need thereof, comprising administering a composition that comprises a p97 conjugate described herein. Also included are methods of delivering an agent to the nervous system (e.g., central nervous system tissues) of a subject, comprising administering a composition that comprises a p97 conjugate described herein. In certain of these and related embodiments, the methods increase the rate of delivery of the agent to the central nervous system tissues, relative, for example, to delivery by a composition that comprises the agent alone.

In some instances, a subject has a disease, disorder, or condition of the CNS, where increased delivery of a therapeutic agent across the blood brain barrier to CNS tissues relative to peripheral tissues can improve treatment, for instance, by reducing side-effects associated with exposure of an agent to peripheral tissues. Exemplary diseases, disorders, and conditions of the CNS include various cancers, including primary and metastatic CNS cancers, lysosomal storage diseases, neurodegenerative diseases such as Alzheimer's disease, and auto-immune diseases such as multiple sclerosis.

Certain embodiments thus relate to methods for treating a cancer of the central nervous system (CNS), optionally the brain, where the subject in need thereof has such a cancer or is at risk for developing such a condition. In some embodiments, the cancer is a primary cancer of the CNS, such as a primary cancer of the brain. For instance, the methods can be for treating a glioma, meningioma, pituitary adenoma, vestibular schwannoma, primary CNS lymphoma, or primitive neuroectodermal tumor (medulloblastoma). In some embodiments, the glioma is an astrocytoma, oligodendroglioma, ependymoma, or a choroid plexus papilloma. In certain embodiments, the primary CNS or brain cancer is glioblastoma multiforme, such as a giant cell gliobastoma or a gliosarcoma.

In particular embodiments, the cancer is a metastatic cancer of the CNS, for instance, a cancer that has metastasized to the brain. Examples of such cancers include, without limitation, breast cancers, lung cancers, genitourinary tract cancers, gastrointestinal tract cancers (e.g., colorectal cancers, pancreatic carcinomas), osteosarcomas, melanomas, head and neck cancers, prostate cancers (e.g., prostatic adenocarcinomas), and lymphomas. Certain embodiments thus include methods for treating, inhibiting or preventing metastasis of a cancer by administering to a patient a therapeutically effective amount of a herein disclosed conjugate (e.g., in an amount that, following administration, inhibits, prevents or delays metastasis of a cancer in a statistically significant manner, i.e., relative to an appropriate control as will be known to those skilled in the art). In particular embodiments, the subject has a cancer that has not yet metastasized to the central nervous system, including one or more of the above-described cancers, among others known in the art.

In particular embodiments, the cancer (cell) expresses or overexpresses one or more of Her2/neu, B7H3, CD20, HerI/EGF receptor(s), VEGF receptor(s), PDGF receptor(s), CD30, CD52, CD33, CTLA-4, or tenascin.

Also included is the treatment of other cancers, including breast cancer, prostate cancer, gastrointestinal cancer, lung cancer, ovarian cancer, testicular cancer, head and neck cancer, stomach cancer, bladder cancer, pancreatic cancer, liver cancer, kidney cancer, squamous cell carcinoma, melanoma, non-melanoma cancer, thyroid cancer, endometrial cancer, epithelial tumor, bone cancer, or a hematopoietic cancer. Hence, in certain embodiments, the cancer cell being treated by a p97 conjugate overexpresses or is associated with a cancer antigen, such as human Her2/neu, HerI/EGF receptor (EGFR), Her3, A33 antigen, B7H3, CDS, CD19, CD20, CD22, CD23 (IgE Receptor), C242 antigen, ST4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-IC, tenascin, vimentin, insulin-like growth factor 1 receptor (IGF-IR), alpha-fetoprotein, insulin-like growth factor 1 (IGF-1), carbonic anhydrase 9 (CA-IX), carcinoembryonic antigen (CEA), integrin av 3, integrin a5 1, folate receptor 1, transmembrane glycoprotein NMB, fibroblast activation protein alpha (FAP), glycoprotein 75, TAG-72, MUCI, MUC16 (or CA-125), phosphatidylserine, prostate-specific membrane antigen (PMSA), NR-LU-13 antigen, TRAIL-RI, tumor necrosis factor receptor superfamily member 10b (TNFRSFIOB or TRAIL-R2), SLAM family member 7 (SLAMF7), EGP40 pancarcinoma antigen, B-cell activating factor (BAFF), platelet-derived growth factor receptor, glycoprotein EpCAM (17-IA), Programmed Death-1, protein disulfide isomerase (POI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and/or mesothelin.

The use of p97 conjugates for treating cancers including cancers of the CNS can be combined with other therapeutic modalities. For example, a composition comprising a p97 conjugate can be administered to a subject before, during, or after other therapeutic interventions, including symptomatic care, radiotherapy, surgery, transplantation, immunotherapy, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

In specific combination therapies, the antibody portion of an p97-antibody conjugate comprises cetuximab, and the p97-cetuximab conjugate is used for treating a subject with locally or regionally advanced squamous cell carcinoma of the head and neck in combination with radiation therapy. In other aspects, the p97-cetuximab conjugate is used for treating a subject with recurrent locoregional disease or metastatic squamous cell carcinoma of the head and neck in combination with platinum-based therapy with 5-fluorouracil (5-FU). In some aspects, the p97-cetuximab conjugate is used in combination with irinotecan for treating a subject with EGFR-expressing colorectal cancer and that is refractory to irinotecan-based chemotherapy.

In some instances, the subject has or is at risk for having a lysosomal storage disease. Certain methods thus relate to the treatment of lysosomal storage diseases in a subject in need thereof, optionally those lysosomal storage diseases associated with the central nervous system. Exemplary lysosomal storage diseases include aspartylglucosaminuria, cholesterol ester storage disease, Wolman disease, cystinosis, Danon disease, Fabry disease, Farber lipogranulomatosis, Farber disease, fucosidosis, galactosialidosis types 1/11, Gaucher disease types 1/11/111, Gaucher disease, globoid cell leucodystrophy, Krabbe disease, glycogen storage disease II, Pompe disease, GMI-gangliosidosis types 1/11/111, GM2-gangliosidosis type I, Tay Sachs disease, GM2-gangliosidosis type II, Sandhoff disease, GM2-gangliosidosis, a-mannosidosis types 1/11, -mannosidosis, metachromatic leucodystrophy, mucolipidosis type I, sialidosis types 1/11 mucolipidosis types 11/1111-cell disease, mucolipidosis type IIIC pseudo-Hurler polydystrophy, mucopolysaccharidosis type I, mucopolysaccharidosis type II, Hunter syndrome, mucopolysaccharidosis type IIIIA, Sanfilippo syndrome, mucopolysaccharidosis type IIIB, mucopolysaccharidosis type IIIC, mucopolysaccharidosis type 111D, mucopolysaccharidosis type IVA, Morquio syndrome, mucopolysaccharidosis type IVB Morquio syndrome, mucopolysaccharidosis type VI, mucopolysaccharidosis type VII, Sly syndrome, mucopolysaccharidosis type IX, multiple sulfatase deficiency, neuronal ceroid lipofuscinosis, CLNI Batten disease, Niemann-Pick disease types NB, Niemann-Pick disease, Niemann-Pick disease type CI, Niemann-Pick disease type C2, pycnodysostosis, Schindler disease types 1/11, Schindler disease, and sialic acid storage disease. In these and related embodiments, the p97 polypeptide can be conjugated to one or more polypeptides associated with a lysosomal storage disease, as described herein.

In certain instances, the subject has or is at risk for having an auto-immune disorder and/or a neurodegenerative disorder, optionally of the CNS. Hence, also included are methods of treating a degenerative or autoimmune disorder of the central nervous system (CNS) in a subject in need thereof. For instance, in specific embodiments, the degenerative or autoimmune disorder of the CNS is Alzheimer's disease, Huntington's disease, Parkinson's disease, or multiple sclerosis (MS). Hence, certain embodiments include administering a p97 conjugate to a subject having Alzheimer's disease, Huntington's disease, Parkinson's disease, or MS. In particular embodiments, the p97 polypeptide is conjugated to an antibody or other agent that specifically binds to amyloid for Alzheimer's Disease, Huntingtin for Huntington's Disease, a-synuclein for Parkinson's Disease, or a4 integrin, CD25, or IL-23 for MS. In some embodiments, the p97 polypeptide is conjugated to an interferon-polypeptide, for the treatment of MS. In specific embodiments, the p97 polypeptide is conjugated to daclizumab for the treatment of MS.

Also included are methods of treating pain in a subject in need thereof. Examples include acute pain, chronic pain, and neuropathic pain, including combinations thereof. In some aspects, the pain has a centrally-acting component, such as central pain syndrome (CPS), where the pain is associated with damage to or dysfunction of the CNS, including the brain, brainstem, and/or spinal cord. In particular embodiments, the p97 polypeptide is conjugated to an antibody or other agent that specifically binds to NGF or TrkA. In specific embodiments, the p97 polypeptide is conjugated to tanezumab for the treatment of pain, optionally for the treatment of osteoarthritis of the knee or hip, chronic low back pain, bone cancer pain, or interstitial cystitis.

Also included are methods of treating inflammation or an inflammatory condition in a subject in need thereof. “Inflammation” refers generally to the biological response of tissues to harmful stimuli, such as pathogens, damaged cells (e.g., wounds), and irritants. The term “inflammatory response” refers to the specific mechanisms by which inflammation is achieved and regulated, including, merely by way of illustration, immune cell activation or migration, cytokine production, vasodilation, including kinin release, fibrinolysis, and coagulation, among others described herein and known in the art. Ideally, inflammation is a protective attempt by the body to both remove the injurious stimuli and initiate the healing process for the affected tissue or tissues. In the absence of inflammation, wounds and infections would never heal, creating a situation in which progressive destruction of the tissue would threaten survival. On the other hand, excessive or chronic inflammation may associate with a variety of diseases, such as hay fever, atherosclerosis, and rheumatoid arthritis, among others described herein and known in the art.

p97 conjugates of the invention may modulate acute inflammation, chronic inflammation, or both. Depending on the needs of the subject, certain embodiments relate to reducing acute inflammation or inflammatory responses, and certain embodiments relate to reducing chronic inflammation or chronic inflammatory responses.

Acute inflammation relates to the initial response of the body to presumably harmful stimuli and involves increased movement of plasma and leukocytes from the blood into the injured tissues. It is a short-term process, typically beginning within minutes or hours and ending upon the removal of the injurious stimulus. Acute inflammation may be characterized by any one or more of redness, increased heat, swelling, pain, and loss of function. Redness and heat are due mainly to increased blood flow at body core temperature to the inflamed site, swelling is caused by accumulation of fluid, pain is typically due to release of chemicals that stimulate nerve endings, and loss of function has multiple causes.

Acute inflammatory responses are initiated mainly by local immune cells, such as resident macrophages, dendritic cells, histiocytes, Kuppfer cells and mastocytes. At the onset of an infection, burn, or other injuries, these cells undergo activation and release inflammatory mediators responsible for the clinical signs of inflammation, such as vasoactive amines and eicosanoids. Vasodilation and its resulting increased blood flow cause the redness and increased heat. Increased permeability of the blood vessels results in an exudation or leakage of plasma proteins and fluid into the tissue, which creates swelling. Certain released mediators such as bradykinin increase sensitivity to pain, and alter the blood vessels to permit the migration or extravasation of leukocytes, such as neutrophils, which typically migrate along a chemotactic gradient created by the local immune cells.

Acute inflammatory responses also includes one or more acellular biochemical cascade systems, consisting of preformed plasma proteins modulate, which act in parallel to initiate and propagate the inflammatory response. These systems include the complement system, which is mainly activated by bacteria, and the coagulation and fibrinolysis systems, which are mainly activated by necrosis, such as the type of tissue damage that is caused by certain infections, burns, or other trauma. Hence, p97 conjugates may be used to modulate acute inflammation, or any of one or more of the individual acute inflammatory responses.

Chronic inflammation, a prolonged and delayed inflammatory response, is characterized by a progressive shift in the type of cells that are present at the site of inflammation, and often leads to simultaneous or near simultaneous destruction and healing of the tissue from the inflammatory process. At the cellular level, chronic inflammatory responses involve a variety of immune cells such as monocytes, macrophages, lymphocytes, plasma cells, and fibroblasts, though in contrast to acute inflammation, which is mediated mainly by granulocytes, chronic inflammation is mainly mediated by mononuclear cells such as monocytes and lymphocytes. Chronic inflammation also involves a variety of inflammatory mediators, such as IFN-y and other cytokines, growth factors, reactive oxygen species, and hydrolytic enzymes. Chronic inflammation may last for many months or years, and may result in undesired tissue destruction and fibrosis.

Clinical signs of chronic inflammation are dependent upon duration of the illness, inflammatory lesions, cause and anatomical area affected. (see, e.g., Kumar et al., Robbins Basic Pathology-8th Ed., 2009 Elsevier, London; Miller, LM, Pathology Lecture Notes, Atlantic Veterinary College, Charlottetown, PEI, Canada). Chronic inflammation is associated with a variety of pathological conditions or diseases, including, for example, allergies, Alzheimer's disease, anemia, aortic valve stenosis, arthritis such as rheumatoid arthritis and osteoarthritis, cancer, congestive heart failure, fibromyalgia, fibrosis, heart attack, kidney failure, lupus, pancreatitis, stroke, surgical complications, inflammatory lung disease, inflammatory bowel disease, atherosclerosis, and psoriasis, among others described herein and known in the art. Hence, p97 conjugates may be used to treat or manage chronic inflammation, modulate any of one or more of the individual chronic inflammatory responses, or treat any one or more diseases or conditions associated with chronic inflammation.

In certain embodiments, p97 conjugates may modulate inflammatory responses at the cellular level, such as by modulating the activation, inflammatory molecule secretion (e.g., cytokine or kinin secretion), proliferation, activity, migration, or adhesion of various cells involved in inflammation.

Examples of such cells include immune cells and vascular cells. Immune cells include, for example, granulocytes such as neutrophils, eosinophils and basophils, macrophages/monocytes, lymphocytes such as B-cells, killer T-cells (i.e., CD8+ T-cells), helper T-cells (i.e., CD4+ T-cells, including ThI and Th2 cells), natural killer cells, yo T-cells, dendritic cells, and mast cells. Examples of vascular cells include smooth muscle cells, endothelial cells, and fibroblasts. Also included are methods of modulating an inflammatory condition associated with one or more immune cells or vascular cells, including neutrophil-mediated, macrophage-mediated, and lymphocyte-mediated inflammatory conditions.

In certain embodiments, p97 conjugates may modulate the levels or activity of inflammatory molecules, including plasma-derived inflammatory molecules and cell-derived inflammatory molecules. Included are pro-inflammatory molecules and anti-inflammatory molecules. Examples of plasma-derived inflammatory molecules include, without limitation, proteins or molecules of any one or more of the complement system, kinin system, coagulation system, and the fibrinolysis system. Examples of members of the complement system include CI, which exists in blood serum as a molecular complex containing about 6 molecules of CIq, 2 molecules of CIr, and 2 molecules of CIs, C2 (a and b), C3 (a and B), C4 (a and b), CS, and the membrane attack complex of CSa, CSb, C6, C7, C8, and C9. Examples of the kinin system include bradykinin, kallidin, kallidreins, carboxypeptidases, angiotensin-converting enzyme, and neutral endopeptidase.

Examples of cell-derived inflammatory molecules include, without limitation, enzymes contained within lysosome granules, vasoactive amines, eicosanoids, cytokines, acute-phase proteins, and soluble gases such as nitric oxide. Vasoactive amines contain at least one amino group, and target blood vessels to alter their permeability or cause vasodilation. Examples of vasoactive amines include histamine and serotonin. Eicosanoids refer to signaling molecules made by oxidation of twenty-carbon essential fatty acids, and include prostaglandins, prostacyclins, thromboxanes, and leukotrienes.

p97 conjugates may also modulate levels or activity of acute-phase proteins. Examples of acute-phase proteins include C-reactive protein, serum amyloid A, serum amyloid P, and vasopressin. In certain instances, expression of acute-phase proteins can cause a range of undesired systemic effects including amyloidosis, fever, increased blood pressure, decreased sweating, malaise, loss of appetite, and somnolence. Accordingly, p97 conjugates may modulate the levels or activity of acute-phase proteins, their systemic effects, or both.

In certain embodiments, p97 conjugates reduce local inflammation, systemic inflammation, or both. In certain embodiments, p97 conjugates may reduce or maintain (i.e., prevent further increases) local inflammation or local inflammatory responses. In certain embodiments, p97 conjugates may reduce or maintain (i.e., prevent further increases) systemic inflammation or systemic inflammatory responses.

In certain embodiments, the modulation of inflammation or inflammatory responses can be associated with one or more tissues or organs. Non-limiting examples of such tissues or organs include skin (e.g., dermis, epidermis, subcutaneous layer), hair follicles, nervous system (e.g., brain, spinal cord, peripheral nerves, meninges including the dura mater, arachnoid mater, and pia mater), auditory system or balance organs (e.g., inner ear, middle ear, outer ear), respiratory system (e.g., nose, trachea, lungs), gastroesophogeal tissues, the gastrointestinal system (e.g., mouth, esophagus, stomach, small intestines, large intestines, rectum), vascular system (e.g., heart, blood vessels and arteries), liver, gallbladder, lymphatic/immune system (e.g., lymph nodes, lymphoid follicles, spleen, thymus, bone marrow), uro-genital system (e.g., kidneys, ureter, bladder, urethra, cervix, Fallopian tubes, ovaries, uterus, vulva, prostate, bulbourethral glands, epidiymis, prostate, seminal vesicles, testicles), musculoskeletal system (e.g., skeletal muscles, smooth muscles, bone, cartilage, tendons, ligaments), adipose tissue, mammaries, and the endocrine system (e.g., hypothalamus, pituitary, thyroid, pancreas, adrenal glands). Accordingly, p97 conjugates may be used to modulate inflammation associated with any of these tissues or organs, such as to treat conditions or diseases that are associated with the inflammation of these tissues or organs.

In particular embodiments, the inflammatory condition has a nervous system or central nervous system component, including inflammation of the brain, spinal cord, and/or the meninges. In particular embodiments, the inflammatory condition of the CNS in meningitis (e.g., bacteria, viral), encephalitis (e.g., caused by infection or autoimmune inflammation such as Acute Disseminated Enchephalomyelitis), sarcoidosis, non-metastatic diseases associated with neoplasia. Particular examples of nervous system or CNS associated inflammatory conditions include, without limitation, meningitis (i.e., inflammation of the protective membranes covering the brain and spinal cord), myelitis, encaphaloymyelitis (e.g., myalgic encephalomyelitis, acute disseminated encephalomyelitis, encephalomyelitis disseminata or multiple sclerosis, autoimmune encephalomyelitis), arachnoiditis (i.e., inflammation of the arachnoid, one of the membranes that surround and protect the nerves of the central nervous system), granuloma, drug-induced inflammation or meningitis, neurodegenerative diseases such as Alzheimer's disease, stroke, HIV-dementia, encephalitis such viral encephalitis and bacterial encephalitis, parasitic infections, inflammatory demyelinating disorders, and auto-immune disorders such as CD8+ T Cell-mediated autoimmune diseases of the CNS. Additional examples include Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-para neoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis, stiff-man syndrome, stroke, traumatic brain injury (TBI), spinal stenosis, acute spinal cord injury, and spinal cord compression.

As noted above, also included is inflammation associated with infections of the nervous system or CNS. Specific examples of bacterial infections associated with inflammation of the nervous system include, without limitation, streptococcal infection such as group B streptococci (e.g., subtypes Ill) and Streptococcus pneumoniae (e.g., serotypes 6, 9, 14, 18 and 23), Escherichia coli (e.g., carrying KI antigen), Listeria monocytogenes (e.g., serotype IVb), neisserial infection such as Neisseria meningitidis (meningococcus), staphylococcal infection, heamophilus infection such as Haemophilus influenzae type B, Klebsiella, and Mycobacterium tuberculosis. Also included are infections by staphylococci and pseudomonas and other Gram-negative bacilli, mainly with respect to trauma to the skull, which gives bacteria in the nasal cavity the potential to enter the meningeal space, or in persons with cerebral shunt or related device (e.g., extraventricular drain, Ommaya reservoir). Specific examples of viral infections associated with inflammation of the nervous system include, without limitation, enteroviruses, herpes simplex virus type 1 and 2, human T-lymphotrophic virus, varicella zoster virus (chickenpox and shingles), mumps virus, human immunodeficiency virus (HIV), and lymphocytic choriomeningitis virus (LCMV). Meningitis may also result from infection by spirochetes such as Treponema pallidum (syphilis) and Borrelia burgdorferi (Lyme disease), parasites such as malaria (e.g., cerebral malaria), fungi such as Cryptococcus neoformans, and ameoba such as Naegleria fowleri.

Meningitis or other forms of nervous system inflammation may also associate with the spread of cancer to the meninges (malignant meningitis), certain drugs such as non-steroidal anti-inflammatory drugs, antibiotics and intravenous immunoglobulins, sarcoidosis (or neurosarcoidosis), connective tissue disorders such as systemic lupus erythematosus, and certain forms of vasculitis (inflammatory conditions of the blood vessel wall) such as Beh<;:et's disease. Epidermoid cysts and dermoid cysts may cause meningitis by releasing irritant matter into the subarachnoid space. Accordingly, p97 conjugates may be used to treat or manage any one or more of these conditions.

As noted above, certain subjects are about to undergo, are undergoing, or have undergone therapy with an otherwise cardiotoxic agent, that is, an agent that displays cardiotoxicity in its unconjugated form (an agent that is not conjugated to p97). Such subjects can benefit from administration of a p97-agent conjugate, relative to administration of the agent alone, partly because p97 can exert a cardioprotective effect on otherwise cardiotoxic agents by a mechanism that is believed to differ from its BBB transport properties. Hence, such subjects can be treated with a p97-cardiotoxic agent conjugate for a variety of disease conditions, including diseases of the CNS described herein, and diseases relating to peripheral, non-CNS tissues.

Exemplary cardiotoxic agents are described elsewhere herein, and can be identified according to well-known in vivo diagnostic and in vitro screening techniques. See Bovelli et al., 2010, supra; Inoue et al., AATEX14, Special Issue, 457-462, 2007; and Dorr et al., Cancer Research. 48:5222-5227, 1988.

For instance, subjects undergoing therapy with a suspected cardiotoxic agent can be monitored by imaging techniques to asses LV systolic and diastolic dysfunction, heart valve disease, pericarditis and pericardial effusion, and carotid artery lesions. LV fractional shortening and LVEF are the most common indexes of LV systolic function for cardiac function assessment, for instance, during chemotherapy. Also, Doppler-derived diastolic indexes represent an early sign of LV dysfunction in patients undergoing therapy, so that evaluation of mitral diastolic flow pattern, early peak flow velocity to atrial peak flow velocity (E/A) ratio, deceleration time of E wave and isovolumic relaxation time can be useful to detect diastolic changes of LV function before systolic dysfunction occurs. Pulsed tissue Doppler may be performed during a standard Doppler echocardiographic examination; it can be reliable in providing quantitative information on myocardial diastolic relaxation and systolic performance (E′ wave, A′ wave and S wave velocity). Tissue Doppler of LV lateral mitral annulus has a recognized prognostic role and, in combination with PW Doppler of mitral inflow, provides accurate information about the degree of LV filling pressure. Early changes in LV myocardial function have been identified by pulsed tissue Doppler of multiple LV sites, and can be relevant determinants of cardiotoxicity.

In particular embodiments, the cardiotoxic agent is a chemotherapeutic, and the subject has cancer. Specific examples of cancers include, without limitation, breast cancers, prostate cancers, gastrointestinal cancers, lung cancers, ovarian cancers, testicular cancers, head and neck cancers, stomach cancers, bladder cancers, pancreatic cancers, liver cancers, kidney cancers, squamous cell carcinomas, CNS or brain cancers (described herein), melanomas, non-melanoma cancers, thyroid cancers, endometrial cancers, epithelial tumors, bone cancers, and hematopoietic cancers.

In specific embodiments, the subject has a Her2/neu-expressing cancer, such as a breast cancer, ovarian cancer, stomach cancer, aggressive uterine cancer, or metastatic cancer, such as a metastatic CNS cancer, and the p97 polypeptide is conjugated to trastuzumab. Such patients can benefit not only from the therapeutic synergism resulting from the combination of p97 and trastuzumab, especially for CNS cancers, but also from the reduced cardiotoxicity of trastuzumab, resulting from the potential cardioprotective effects of p97.

In some instances, the subject has a disease related to IL-1. IL-1 is a master regulator of inflammation via controlling a variety of innate immune processes. There are two individual forms of IL-1, IL-1α and IL-1β, but they are indistinguishable in terms of their biological functions. Although the homology between IL-1α and IL-1β is not high in terms of amino acid sequences, IL-1α and IL-1β are structurally similar and show the same functions by sharing a common receptor, IL-1 type 1 receptor (IL-1R1), and both have the same central-barrel along with adjoining loops. The difference between IL-1α and IL-1β is an N-terminal extension of 14 residues beyond the N-terminus of IL-1α and IL-1β.

There are two cell surface IL-1 receptors (IL-1R1) and IL-1 type 2 receptor (IL-1R2), a decoy receptor. IL-1 binds to IL-1R1, which requires the formation of a heterodimer with the IL-1 type 3 receptor (IL-1R3) (also known as IL-1RAcP) accompanied by adaptor IL-1 receptor-associated kinase (IRAK) and myeloid differentiation primary response protein 88 (MyD88). IL-1R1 initiates inflammatory responses when binding to the ligands IL-1α and IL-1β and has been reported to be expressed by Tlymphocytes, fibroblasts, epithelial cells, and endothelial cells. IL-1R2, which does not initiate signal transduction, is expressed in a variety of hematopoietic cells, especially in B-lymphocytes, mononuclear phagocytes, polymorphonuclear leukocytes, and bone marrow cells.

IL-1α and IL-1β are expressed in a wide range of tissues and a variety of cells, especially in macrophages in lymphoid organs including the thymus, spleen, lymph nodes, Peyer's patches, and bone marrow. In non-lymphoid organs, IL-1α and IL-1β are expressed in tissue macrophages in the lung, digestive tract, and liver. They are also expressed in cellular subepithelial endometrial tissue of the uterus, in the glomeruli, in outer cortical areas of the kidney, and in various specific cell types, including neutrophils, keratinocytes, epithelial and endothelial cells, lymphocytes, smooth muscle cells, and fibroblasts.

IL-1 has been known as associating a number of diseases, including autoinflammatory diseases, metabolic syndromes, chronic inflammation and malignancy, fatigue in associated with various diseases, and etc.

Autoinflammatory diseases, generally caused by single nucleotide mutation of the CIAS1 gene results in NLRP3 mutation which induces constituted inflammasome activation causing cryopyrin-associated periodic syndrome (CAPS), includes familial cold autoinflammatory syndrome (FCAS), Muckle-Wells syndrome (MWS), and neonatal-onset multisystem inflammatory disease (NOMID)/chronic infantile neurologic, cutaneous, and arthritis (CINCA) syndrome, which leads to greater IL-1p secretion without any DAMPs or PAMPs. The most common autoinflammatory disease is Familial Mediterranean fever (FMF). Inflammatory diseases like those above, characterized by the enhanced secretion of IL-1p, include a group of autoinflammatory diseases such as NLRP12 autoinflammatory syndrome; hyperimmunoglobulinemia D and periodic fever syndrome (HIDS)/mevalonate kinase deficiency (MKD); pyogenic arthritis, pyoderma gangrenosum, and acne (PAPA) syndrome; pyoderma gangrenosum, acne, and suppurative hidradenitis (PASH) syndrome; pyogenic arthritis, acne, pyoderma gangrenosum, and suppurative hidradenitis (PAPASH); Majeed syndrome; and TNF-receptor-1-associated syndrome (TRAPS). Other autoinflammatory diseases associated with IL-1 includes schnitzler syndrome, systemic-onset juvenile idiopathic arthritis, adult-onset still disease, acute gout, chronic gout, pseudogout, as well as type 2 diabetes. Regarding the metabolic syndromes, NLRP3 inflammasomes have also been reported to be involved in low-grade subclinical inflammation induced by chronic exposure to high levels of free fatty acids and glucose, leading to increased apoptosis and impaired insulin secretion of β-cells in obese type 2 diabetes mellitus (T2D) patients. Islet amyloid polypeptide (IAPP) oligomers activated NLRP3 inflammasomes to induce significant IL-1β production by infiltrating macrophages in an in vivo study. Higher concentrations of glucose activate NF-κB and IL-1 precursors in cells. Minimally oxidized low-density lipoproteins stimulate TLR4, which triggers IL-1β expression. Another oligomer of amyloid, amyloid P, can induce IL-1β via NLRP3 inflammasomes in a process involving the phagocytosis of amyloid p in glial cells in patients with Alzheimer's disease (AD) and subsequent lysosomal damage and release of cathepsin B. ROS are considered to be involved in the activation of NLRP3 inflammasomes, and it was suggested that direct interaction between amyloidogenic peptide and NLRP3 could initiate NLRP3 inflammasome formation in a cell-free system. Both IL-1α and IL-1β gene polymorphisms have been reported to be associated with central obesity and metabolic syndrome in a population with coronary heart disease in an epidemiologic study. Thus, these diseases are IL-1-dependent cytokinopathies (interleukinoneopathies)

IL-1β plays a role in resolving acute inflammation resulting in the initiation of adaptive anti-tumor responses; however, chronic inflammatory conditions increase the risk of developing cancer. In human breast cancer, higher expression of IL-1p is associated with tumor invasiveness and aggressive tumor biology. Expression of IL-1α, IL-1μ, and their receptors in human breast cancer tissues results in the activation of a population of cells and subsequently contributes to angiogenesis, tumor proliferation, and tumor invasion in the microenvironment.

Knowing the above mentioned associations between the diseases and the IL-1, application of IL-1 blockade are useful in treatment of the diseases.

For example, with respect to autoinflammatory diseases, the recombinant human IL-1-receptor antagonist anakinra is markedly effective against CAPS such as MWS, FCAS, and NOMID/CINCA. In several case reports of patients with FMF, anti-IL-1 treatment with anakinra, canakinumab, or rilonacept in colchicine-resistant patients was highly effective Rapid and lasting response with anakinra treatment in a patient with PAPA syndrome. In addition, Anakinra and canakinumab therapies were also reported to be effective in patients with MKD/HIDS. Anakinra monotherapy is also significantly effective for patients with adult-onset still's disease, especially in prednisone-resistant patients. Canakinumab and anakinra are also effective for patients with Schnitzler syndrome. In the case of TRAPS, although TNF-α is considered to be mainly involved in clinical manifestations, marked improvement following IL-1β blockade occurred.

In field of autoimmune diseases, Anakinra are successful in treating patients with psoriatic arthritis, ankylosing spondylitis, and rheumatoid arthritis.

IL-1 blockade is also proven to be effective in treating infectious diseases. In particular, anakinra is safe and may be associated with dose-related survival advantage in patients with septic shock syndrome complicated by acute respiratory distress syndrome, disseminated intravascular coagulation, and renal dysfunction, and subsequent secondary hemophagocytic lymphohistiocytosis (HLH), or macrophage-activating syndrome (MSA). For sepsis with MSA, anakinra treatment leads to significant improvements in hepatobiliary dysfunction and disseminated intravascular coagulation in patients.

Regarding the metabolic syndrome, IL-1 inhibition by anakinra, rilonacept or canakinumab is efficacious for treating gout patients. Regarding the ischemic diseases, such as Myocardial infraction (MI), stroke, or cerebral infarction, or tissue injury, cell death by necrosis takes place and the IL-1α precursor is released in sterile inflammation. Animal studies showed that inhibition of IL-1 is effective in limiting atherosclerosis and cardiovascular events and improving symptoms of acute myocardial infarction and ischemic stroke.

In field of malignant diseases, IL-1p is thought to play an important role in cancer invasiveness, progression, and metastases via inflammation in the tumor microenvironment. Antiflammatory therapy with canakinumab targeting IL-1β innate immunity pathway could significantly reduce lung cancer mortality, while treatment of metastatic breast cancer-related with anakinra therapy eliminates a systemic transcriptional signature of IL-1 associate inflammation in blood cells.

Except for the abovementioned diseases, IL-1 has recently been shown as associated with fatigue, in particular, IL-1 acts as a mediator of fatigue in diseases. The central nervous system (CNS) plays an important role in cytokine-induced fatigue. Elevation of IL-1 in the brain contributes to behavioral alterations described as “sickness behavior,” which includes increased feelings of fatigue and depressed mood, loss of interest in social interactions, and reduction of physical activity both in animals and in humans treated for different malignancies. The observed behavioral alterations in response to the intrathecal administration of pro-inflammatory cytokines indicate that, in addition to its peripheral effect on the immune response, IL-1 also signals to the brain via several immune-to-brain communication pathways.

The IL-1R1 is distributed throughout the brain. The intracellular pathways after IL-1R1 activation in the brain are similar to those in the periphery, eventually leading to NFκB activation and subsequent gene transcription. In an animal experiment, an increase of IL-1β messenger RNA (mRNA) was found in the hypothalamus directly after peripheral injection of IL-1μ, where it is able to induce fever. While the concentration in the hypothalamus decreased within 24 hours, upregulation of IL-1β mRNA persisted in the cerebral cortex, and this was accompanied by a decrease in spontaneous activity lasting several days. Hypothetically, such persistence of IL-1β transcription might be due to epigenetic changes in microglial cells, a process that is thought to play a role in several neuroinflammatory disorders.

Besides their effects on brain neurotransmitter systems, IL-1 can also influence brain functioning through their effect on hippocampal neuroplasticity and neurogenesis or via neuro-endocrine mechanisms involving the hypothalamic pituitary-adrenal axis (HPA) functioning. These effects have been associated with the development of mental problems that often concur with fatigue symptoms, such as impairments in learning and memory and depressive-like behavior.

In area of inflammatory diseases, IL-1 is related to Rheumatoid arthritis (RA), sjögren's syndrome, cryopyrin-associated periodic syndrome, sarcoidosis, and etc. RA is a chronic disease characterized by recurrent, often symmetrical destructive arthritis. In addition to local joint inflammation, RA is known for systemic symptoms such as fatigue. Both concentrations of IL-1β and IL-1Ra are slightly elevated in RA, and both correlate with disease severity, reflected by elevated pain scores and an increased erythrocyte sedimentation rate (ESR). Sjögren's syndrome is characterized by autoantibody production against ribonucleoparticles and mononuclear cell accumulations in exocrine glands. Besides sicca complaints, fatigue is one of the most frequently noted symptoms in Sjögren's syndrome. Fatigue is also reported by CAPS patients and influence of blocking IL-1 on disease severity and fatigue was assessed in several studies.

In area of non-inflammatory diseases, IL-1 associated fatigues exists in patient having diabetes mellitus, Post-stroke fatigue, Chronic fatigue syndrome, as well as cancers including prostate cancer, breast cancer, testicular cancer, hematologic malignancies and other types of cancers. In type 2 diabetes, a monoclonal anti-IL1β antibody has been proven to be effective in treating fatigue in patients. Regarding cancer, fatigue is one of the most prominent symptoms during all stages of disease, leading to substantial impairment and disability. The exact mechanism causing fatigue during and after cancer treatment is not clear, but it is suspected that pro-inflammatory cytokines, especially TNF-α and IL-1β play an important role.

Other diseases that are associated with IL-1 includes neuropathic pain, fabry disease, epilepsy and seizure, fabrile disease, fabrile infection-related epilepsy syndrome, fabrile status epilepticus, generalised epilepsy with febrile seizures plus, dravet syndrome, Idiopathic hemiconvulsion hemiplegia and epilepsy syndrome, Parkinson's disease, IL-1 receptor antagonist deficiency syndrome, and new-onset refractory status epilepticus.

Accordingly, in some specific embodiments, the subject has at least one of IL-1 related diseases such as autoinflammatory diseases, autoimmune disease, metabolic syndromes, chronic inflammation and malignancy, infectious diseases, ischemic diseases, fatigues associated with inflammatory and non-inflammatory diseases, neuropathic pain, fabry disease, epilepsy and seizure, fabrile disease, fabrile infection-related epilepsy syndrome, fabrile status epilepticus, generalised epilepsy with febrile seizures plus, dravet syndrome, Idiopathic hemiconvulsion hemiplegia and epilepsy syndrome, Parkinson's disease, IL-1 receptor antagonist deficiency syndrome, and new-onset refractory status epilepticus. In particular embodiments, the aforementioned condition has a nervous system or central nervous system component.

Accordingly, p97 conjugates may be used to treat or manage any one or more of these IL-1 related conditions. In specific embodiments, p97 conjugate may comprise a p97 polypeptide and a IL-1 receptor atagonist, either directly or indirectly via a linker. In other embodiment, p97 polypeptide and IL-1 receptor antagonists may be associated to form a conjugate via chemical conjugation or genetic fusion.

Methods for identifying subjects with one or more of the diseases or conditions described herein are known in the art.

Also included are methods for imaging an organ or tissue component in a subject, comprising (a) administering to the subject a composition comprising a human p97 (melanotransferrin) polypeptide, or a variant thereof, where the p97 polypeptide is conjugated to a detectable entity, and (b) visualizing the detectable entity in the subject, organ, or tissue.

In particular embodiments, the organ or tissue compartment comprises the central nervous system (e.g., brain, brainstem, spinal cord). In specific embodiments, the organ or tissue compartment comprises the brain or a portion thereof, for instance, the parenchyma of the brain.

A variety of methods can be employed to visualize the detectable entity in the subject, organ, or tissue. Exemplary non-invasive methods include radiography, such as fluoroscopy and projectional radiographs, CT-scanning or CAT-scanning (computed tomography (CT) or computed axial tomography (CAT)), whether employing X-ray CT-scanning, positron emission tomography (PET), or single photon emission computed tomography (SPECT), and certain types of magnetic resonance imaging (MRI), especially those that utilize contrast agents, including combinations thereof. Merely by way of example, PET can be performed with positron-emitting contrast agents or radioisotopes such as 18 F, SPECT can be performed with gamma-emitting contrast agents or radioisotopes and MRI can be performed with contrast agents or radioisotopes. Any one or more of these exemplary contrast agents or radioisotopes can be conjugated to or otherwise incorporated into a p97 polypeptide and administered to a subject for imaging purposes.

For instance, p97 polypeptides can be directly labeled with one or more of these radioisotopes, or conjugated to molecules (e.g., small molecules) that comprise one or more of these radioisotopic contrast agents, or any others described herein.

For in vivo use, for instance, for the treatment of human disease, medical imaging, or testing, the conjugates described herein are generally incorporated into a pharmaceutical composition prior to administration. A pharmaceutical composition comprises one or more of the p97 polypeptides or conjugates described herein in combination with a physiologically acceptable carrier or excipient.

To prepare a pharmaceutical composition, an effective or desired amount of one or more of the p97 polypeptides or conjugates is mixed with any pharmaceutical carrier(s) or excipient known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof.

Administration of the polypeptides and conjugates described herein, in pure form or in an appropriate pharmaceutical composition, can be carried out via any of the accepted modes of administration of agents for serving similar utilities. The pharmaceutical compositions can be prepared by combining a polypeptide or conjugate or conjugate-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other anti-cancer agents as described elsewhere herein) and/or suitable excipients such as salts, buffers and stabilizers may, but need not, be present within the composition.

Administration may be achieved by a variety of different routes, including oral, parenteral, nasal, intravenous, intradermal, subcutaneous or topical. Preferred modes of administration depend upon the nature of the condition to be treated or prevented.

Carriers can include, for example, pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In certain aspects, the p97 polypeptide sequence and the agent are each, individually or as a pre-existing conjugate, bound to or encapsulated within a particle, e.g., a nanoparticle, bead, lipid formulation, lipid particle, or liposome, e.g., immunoliposome. For instance, in particular embodiments, the p97 polypeptide sequence is bound to the surface of a particle, and the agent of interest is bound to the surface of the particle and/or encapsulated within the particle. In some of these and related embodiments, the p97 polypeptide and the agent are covalently or operatively linked to each other only via the particle itself (e.g., nanoparticle, liposome), and are not covalently linked to each other in any other way; that is, they are bound individually to the same particle. In other embodiments, the p97 polypeptide and the agent are first covalently or non-covalently conjugated to each other, as described herein (e.g., via a linker molecule), and are then bound to or encapsulated within a particle (e.g., immunoliposome, nanoparticle). In specific embodiments, the particle is a liposome, and the composition comprises one or more p97 polypeptides, one or more agents of interest, and a mixture of lipids to form a liposome (e.g., phospholipids, mixed lipid chains with surfactant properties). In some aspects, the p97 polypeptide and the agent are individually mixed with the lipid/liposome mixture, such that the formation of liposome structures operatively links the p97 polypeptide and the agent without the need for covalent conjugation. In other aspects, the p97 polypeptide and the agent are first covalently or non-covalently conjugated to each other, as described herein, and then mixed with lipids to form a liposome. The p97 polypeptide, the agent, or the p97-agent conjugate may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents, such as cytotoxic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described conjugate in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a p97 polypeptide, agent, or conjugate described herein, for treatment of a disease or condition of interest.

A pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a p97 polypeptide or conjugate as herein disclosed such that a suitable dosage will be obtained. Typically, this amount is at least 0.01% of the agent of interest in the composition. When intended for oral administration, this amount may be varied to be between 0.1 and about 70% of the weight of the composition. Certain oral pharmaceutical compositions contain between about 4% and about 75% of the agent of interest. In certain embodiments, pharmaceutical compositions and preparations according to the present invention are prepared so that a parenteral dosage unit contains between 0.01 to 10% by weight of the agent of interest prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the conjugate or agent and thereby assists in the delivery of the compound. Suitable agents that may act in this capacity include monoclonal or polyclonal antibodies, one or more proteins or a liposome.

The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s).

Delivery of the aerosol includes the necessary container, activators, valves, subcontainers, and the like, which together may form a kit. One of ordinary skill in the art, without undue experimentation may determine preferred aerosols.

The compositions comprising conjugates as described herein may be prepared with carriers that protect the conjugates against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The pharmaceutical compositions may be prepared by methodology well known in the pharmaceutical art. For example, a pharmaceutical composition intended to be administered by injection can be prepared by combining a composition that comprises a conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the conjugate so as to facilitate dissolution or homogeneous suspension of the conjugate in the aqueous delivery system.

The compositions may be administered in a therapeutically effective amount, which will vary depending upon a variety of factors including the activity of the specific compound (e.g., conjugate) employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Generally, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., ˜3.5 g); more preferably a therapeutically effective dose is (for a 70 kg mammal) from about 1 mg/kg (i.e., ˜70 mg) to about 25 mg/kg (i.e., ˜1.75 g).

Compositions comprising the conjugates described herein may also be administered simultaneously with, prior to, or after administration of one or more other therapeutic agents, as described herein. For instance, in one embodiment, the conjugate is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Such combination therapy may include administration of a single pharmaceutical dosage formulation which contains a compound of the invention and one or more additional active agents, as well as administration of compositions comprising conjugates of the invention and each active agent in its own separate pharmaceutical dosage formulation. For example, a conjugate as described herein and the other active agent can be administered to the patient together in a single oral dosage composition such as a tablet or capsule, or each agent administered in separate oral dosage formulations. Similarly, a conjugate as described herein and the other active agent can be administered to the patient together in a single parenteral dosage composition such as in a saline solution or other physiologically acceptable solution, or each agent administered in separate parenteral dosage formulations. Where separate dosage formulations are used, the compositions comprising conjugates and one or more additional active agents can be administered at essentially the same time, i.e., concurrently, or at separately staggered times, i.e., sequentially and in any order; combination therapy is understood to include all these regimens.

### EXEMPLIFICATION

The following examples are provided to illustrate the invention, but are not intended to limit the scope of the claims which follow.

Materials and Methods

The MTf Peptide

The peptide fragment from the trypsin digest of MTf (MTfpep) corresponding to amino acids 441-452 (DSSHAFTLDELR) of the mature protein was previously identified as crossing the in vitro model of the BBB and is utilized in the current study and details of the identification of the MTf peptide is described elsewhere.²¹ Briefly, recombinant human MTf, expressed and purified as previously described by Yang et al.;¹³ Karkan et al.;¹⁹ and Hegedus et al.,²² was subjected to trypsin digest at 37° C. for 20 hours and lyophilized. The tryptic peptides were screened with transcytosis assay using the in vitro BBB model (Cellial Technologies, Lens France) using bovine brain capillary endothelial cells grown on collagen coated polycarbonate transwell inserts (0.4 μm pore size, 24 mm diameter, corning) forming a confluent monolayer supported by primary rat glial cells. Lucifer Yellow (LY) at 20 μM was used as paracellular marker to evaluate the integrity of the cellular barrier. Ringer-HEPES buffer containing the trypsin digest of MTf and LY was placed in the upper chamber (luminal side) of the transwell. The transwells were incubated on a rocking platform at 37° C. for 120 minutes. At the end of the experiment, aliquots were taken from the abluminal and luminal compartments of the transwell and lyophilized and stored at −80° C. until mass spectrometry analysis. Samples from the transwell experiment were resolved by liquid chromatography using Agilent Eclipse Plus C18 column. The eluates were analyzed on an Agilent 6490 QqQ mass spectrometer in positive mode, controlled by Agilent's MassHunter Worstation software (version B.04.01) and processed using Agilent Quantitation software (version B.04.01).

NIP228 hIgG1 Control Antibody

NIP228 hIg1 is a mouse IgG1 kappa monoclonal antibody against 4-hydroxy-3-iodo-5-nitrophenylacetic acid, which is used as a negative control for protein fusion crossing the BBB. When bound to IL-IRA, it previously showed no analgesia in a mouse model of neuropathic pain as it was not able to cross the BBB.²³

Labelling of the NIP228 Antibody and MTf Peptide with a Fluorescent Marker with Alexa Fluor 647 for Confocal Fluorescence Microscopy

The MTfpep was reacted with Alexa Fluor 647 (AF647) C2-maleimide (invitrogen A20347) and purified using semi-preparative reverse phase C18 chromatography following the reaction. NIP228 mouse IgG1 antibody was reacted with Alexa Fluor 647 NHS ester (Invitrogen) and then desalted using a Sephadex G-25 column.

Chemical Conjugation of MTf or MTfpep to NIP228 mAb for Confocal Fluorescence Microscopy

The Alexa Fluor labelling and conjugation was performed by CellMosaic, Inc. Briefly, desalted NIP228 hIgG1 was reacted with N-(β-maleimidopropyloxy) succinimide ester (BMPS). In a separate reaction, MTf was reacted with 2-pyridyldithiol-tetraoxatetradecane-N-hydroxysuccinimide (PEG₄-SPDP) and then desalted. The fractions collected were subjected to dithiothreitol (DTT) reduction to generate MTf-containing free sulfhydryl groups. Sulfhydryl-containing MTf was further reacted with NIP228 hIgG1 (AF647) containing maleimide groups generated from the mAb(AF647)ₙ and BMPS reaction. The reaction was quenched by cysteine, and the crude reaction mixture was purified by size exclusion chromatography. The fractions containing mostly 1:1 MTf and NIP228 hIgG1 conjugate were combined, concentrated and sterilized using a sterile ultrafree MC 0.22 μm GV Durapore spin filter. The resulting conjugate contained approx. 2 MTf per NIP228 antibody.

MTfpep-NIP228 hIgG1 conjugate was prepared in similar fashion, where maleimide-containing NIP228 hIgG1 was reacted with thiol-containing MTfpep. The reaction was quenched by cysteine and desalted. The resulting conjugate contained approx. 4 MTfpep per NIP228 antibody.

The conjugation products were analyzed by either reversed phase or size exclusion HPLC to determine purity and concentration, and by SDS-PAGE to determine purity and molecular weight.

Cloning, expression and purification of MTf and MTfpep-NIP228 fusion constructs DNA encoding the amino acid sequence of the V_{H} and V_{L} of the antibody, NIP228, was assembled by polymerase extension of over-lapping oligonucleotides and cloned into expression vectors containing the appropriate light or heavy chain constant regions.²⁴ DNA encoding the entire MTf or MTfpep amino acid sequence (DSSHAFTLDELR) and (Gly₄Ser)₂₋₄ flexible linkers was similarly assembled by polymerase extension of over-lapping oligonucleotides and directional cloning either to the N or C-terminal end of the acceptor IgG heavy chain. All IgGs were expressed as chimeric human IgG1 molecules with the S239D/A330L/1332E triple mutation (IgG1 TM).²⁵ Antibodies were expressed in transiently transfected Chinese hamster ovary (CHO) cells in serum-free media as described previously.²⁶ Antibodies were purified from cell culture media using protein A affinity chromatography followed by size exclusion chromatography. The concentration of IgG was determined by A₂₈₀ using an extinction coefficient based on the amino acid sequence of the IgG.²⁷

Plasmids enabling the expression of IL-1RA fused to the C-terminus of the IgG1 TM heavy chain via a (G₄S)₃ flexible linker were assembled by PCR amplification of the IL-1RA gene from cDNA obtained from Source Bioscience and subsequent PCR amplification with oligonucleotide primers that overlapped the IL-1RA gene and the IgG1 TM CH3 domain and incorporated the linker, described above. Expression and purification of IgG1 TM-IL-IRA fusions was performed as previously described.

Chemical Conjugation of MTfpep-NIP228 mAb Constructs

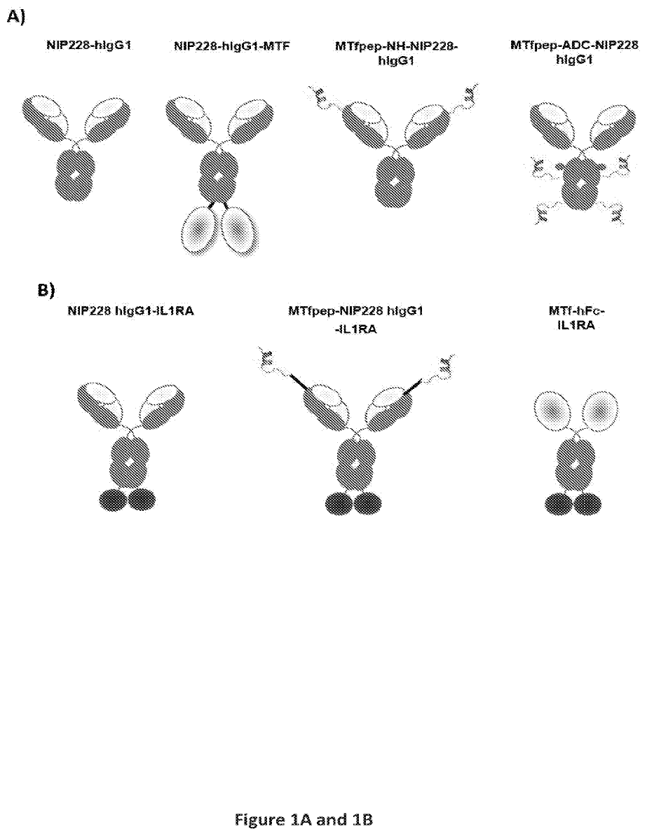

MTfpep was chemically synthetized with a C-terminal polyethylene glycol (PEG)₄ linker followed by a Lysine residue containing a maleimide grouping for linking to the thiol group of engineered cysteines residues on the Fc region of the NIP228 hIgG1TM as described in Thompson et al.²⁸ Briefly, to generate the site-specific peptide conjugation, cysteine engineered NIP228 was reduced using 40 molar equivalent excess of TCEP (Tris(2-carboxyethyl) phosphine; Thermo Scientific Bond-breaker TCEP solution) in PBS pH 7.2, 1 mM EDTA (Ethylenediaminetetraacetic acid) for 3 hours at room temperature. Following buffer exchange to remove TCEP, 20 molar equivalents of dehydroascorbic acid were added for 4 hours at room temperature. The resulting solution was filtered through a 0.2 μm syringe filter and 10 molar equivalents of MTf peptide was added followed by incubation at room temperature for 1 hour. The reaction was quenched by the addition of 4 equivalents (over MTf peptide) of N-acetyl cysteine. The MTf peptide labelled NIP228 hIgG1TM was purified using size exclusion chromatography. The peptide to antibody ratio was estimated as described in Thompson et al.²⁸ To avoid differences in renal clearance molecules containing the MTfpep were constructed on an IgG, rather than only the Fc domain, as the Fab arms increase the molecular weight and hydrodynamic radius reducing clearance through the kidneys. FIG. 1 shows the design and composition of the different molecules based on NIP228, with FIG. 1A showing the molecules used in peripheral pharmacokinetic (PK) and in brain exposition and FIG. 1B showing the molecules having integrated IL-1RA on the C terminus of human Fc which were used in the mouse pharmacodynamic (PD) model of neuropathic pain.

Brain Distribution of MTfpep, mAb and MTfpep/MTf-mAb Chemical Conjugate by Confocal Fluorescence Microscopy

The in-life phase and confocal microscopy studies were performed under contract at the National Research Council (NRC) of Canada, (Ottawa, ON). The protocol and procedures involving the care and use of animals in this study were reviewed and approved by Ottawa-NRC Animal Care Committee. The care and use of animals were in accordance with the guidelines of the Canadian Council on Animal Care (CCAC). The experiments in this study have been reported in compliance with the ARRIVE guidelines (Animal Research: Reporting in Vivo Experiments).

6-8 weeks old Balb/c female mice (n=3) were injected (i.v.) with mAbAF647 (10 mg/kg), MTf-mAbAF647 (15 mg/kg), and MTfpep-mAbAF647 (10.2 mg/kg), the doses representing equivalent moles/kg doses due to the differing molecular weights of the three molecules. The control groups received vehicle control, phosphate buffered saline (PBS), or free AF647 (0.1 mg/kg). 2 hours post injection, tomato lectin Texas red to label the capillaries (100 ag/mouse) was injected (i.v.) prior to euthanasia (10 min). The animals were euthanized by anesthetisation and perfused with PBS pH 7.4 supplemented with 2.7% BSA, 100 U/mL heparin. The brain was removed, frozen and subjected to cryo-sectioning and immunohistochemistry. Tissue sections were stained with 4′,6-diamidino-2-phenylindole (DAPI blue) to label the cell nuclei.

The 3D confocal imaging was performed at the Penn State Hershey College of Medicine Imaging Core Lab. Confocal images of fluorescently labeled cells were acquired with a Leica AOBS SP8 laser scanning confocal microscope (Leica, Heidelberg, Germany) using a high-resolution Leica 63X/1.4 or 40X/1.3 Plan-Apochromat oil immersion objective lenses. The laser lines used for excitation were continuous wave 405 for DAPI, 80 MHz pulsed 595 nm for Texas Red and 80 MHz pulsed 653 nm for AF647. All images and spectral data (except DAPI) were generated using the highly sensitive Leica HyD hybrid detectors (with time gated option) located inside the scan-head. The 3D stack images with optical section thickness (z-axis) of approximately 0.3 μm were captured from tissue volumes. The 3D image restoration was performed using Imaris software (Bitplane). The volume estimation was performed on the 3D image data sets recoded from five or more different areas of brain tissue samples. Gaussian noise removal filter was applied to define the boundary between foreground and background, and the lower threshold level was set to exclude all possible background voxel values. Sum of all the voxels above this threshold level is determined to be volume. 3D image volume of tissues was systematically compared using similar imaging conditions. Distribution of test articles in brain capillaries and parenchyma are quantified as volume fraction, determined as volume of test article (voxels of AF647 co-localized with either capillary or parenchyma) divided by total tissue volume.

ANOVA multiple comparison analysis used for comparison between the volume fraction of AF647 fluorescence associated with NIP228 hIgG1 and MTfpep-NIP228 hIgG1 or MTf-NIP228 hIgG1 in the brain parenchyma and capillaries. All differences were considered statistically significant at p<0.05. Data is reported as Mean±Standard Deviation (SD).

Peripheral Kinetics and Brain Exposure

All studies to measure antibody exposure in the periphery and brain were performed at Quotient Biosciences (Newmarket, UK). Male C57Bl/6 mice, age 10-12 weeks were intravenously (i.v.) injected with MTf and the MTfpep genetically or chemically conjugated variants of the control IgG (NIP228) at 20 mg/kg or molar equivalent. Intravenous doses were administered into a tail vein at a constant dose volume of 10 ml/kg. Antibodies were supplied in D-PBS (Sigma). Following dosing, two blood plasma samples were collected into individual Li-Heparin containers from each of six animals per time point (0.08, 2, 4, 6, 8, 24, 48, 96, 120, 168, 240 & 336 hours), per dose group. The first sample from each animal was collected from the lateral tail vein (ca 200 pL) into a Li-Hep microvette (BD Diagnostic Systems), while the second sample (ca 600 μL) was collected by cardiac puncture under isoflurane anaesthesia into a Li-hep microtainer (BD Diagnostic Systems). Following collection, blood samples were allowed to clot for 30 min and centrifuged at 10,000×g for 2 min at 4° C. and the resultant plasma drawn off. Plasma samples were flash frozen on dry ice for subsequent analysis. After final blood collection, the mice were perfused with D-PBS at a rate of 2 ml/min for 10 min until the extremities appeared white. Brains were excised, and one hemisphere immediately processed, the other snap frozen in liquid nitrogen.

Brain hemisphere was homogenized in 5 volumes of ice-cold PBS containing 0.5% Tween 20 and Complete® protease inhibitor cocktail tablets (Roche Diagnostics). Homogenisation was performed in a 10 ml Potter-Elvehjem mortar type glass homogeniser with polytetrafluoroethylene (PTFE) pestle, using 2×10 clockwise strokes with five sec rest time. Homogenates were transferred to LoBind tubes (Eppendorf) and rotated at 4° C. for 1 hour before centrifuging in a chilled bench-top centrifuge at 13,000 g for 20 min. The supernatant was isolated for brain antibody measurement. Five volumes of ice-cold PBS containing 0.2% sodium dodecyl sulphate (SDS) and Complete® protease inhibitor cocktail tablets were added to the remaining cell pellet and the pellet processed as described above. The supernatant was again removed and combined with the 1^{st} supernatant for measurement via MesoScale Discovery (MSD) assay (Meso Scale Diagnostics). MSD multi-array technology enables detection of biomarkers in single and multiplex formats using electrochemiluminescent detection.

Animal husbandry and the procedures used were in accordance with the guidelines of the AstraZeneca Animal Care Committee and complied with the Animals (Scientific Procedures) Act 1986. All experiments were reported in compliance of the Animal Research: Reporting in Vivo Experiments (ARRIVE) guidelines.

Measurement of Antibody Concentrations in Mouse Brain and Plasma

Antibody concentrations in mouse plasma and brain samples were measured via MSD assay platform. The MSD assay employs a plate-based sandwich immunoassay format where anti-human IgG capture antibody binds calibrator or samples, and a specific detection antibody labelled with SULFO-TAG emits light upon electrochemical stimulation. Levels of MTf-NIP228, MTfpep-NIP228 and control antibody alone+/−IL-1RA fusions in plasma and brain samples were quantified by reference to standard curves generated using calibrator samples with a four-parameter nonlinear regression model.

Non-compartmental pharmacokinetic (PK) analysis (NCA) was performed using Phoenix WinNonlin Professional [version 6.3; Pharsight (Certara), Sunnyvale, Calif.]. Nominal collection times were used for the PK data analyses, with below level of quantification (BLQ) values set to “missing” for calculation of the concentration means at nominal time points. The BLQ values were set as “zero” at pre-dose, and “missing” after peak concentrations for the NCA analysis.

The area under the concentration-time curve to the last measurable time point (AUCₗₐₛₜ) was calculated for plasma and brain using the linear trapezoidal method as implemented in WinNonlin Phoenix. Additionally, systemic clearance (CL), terminal volume of distribution (Vz) and terminal half-life (t_{1/2}) were determined for plasma. The C and T quoted are the observed values based on the mean concentration data at each time point.

Partial Nerve Ligation

Partial nerve ligation was performed in mice as described in Chessell et al.²⁹ and Webster et al.²³ In brief, the left sciatic nerve was exposed in female C57Bl/6J mice (Charles River, UK) by blunt dissection through an incision at the level of the mid-thigh. A suture (9/0 Virgin Silk: Ethicon) was then passed through the dorsal third of the nerve and tied tightly. The mice were allowed to recover for at least seven days prior to commencement of testing. Sham operated mice underwent the same protocol but following exposure of the nerve the mice were allowed to recover. Mice were tested for baseline responses on day 7 and day 10 post surgery. Operated mice showing ipsilateral/contralateral ratios of greater than 80% were classed as non-responders and were removed from the study. The remaining mice were then randomly allocated into treatment groups of 8-10 mice per group with approximately equal ipsilateral/contralateral ratios following which mice were treated with the compound under test. Separate animals were used in each study. All procedures were performed in accordance with the Animals (Scientific Procedures) Act 1986 and were approved by a local ethics committee. Following the establishment of baseline readings mice were divided into 2 groups with approximately equal ipsilateral/contralateral ratios which underwent surgery to partially ligate the sciatic nerve or served as sham operated controls based on the previously described method of Seltzer et al.³⁰ To investigate the effects of the or MTf or MTfpep fusions animals received one of PBS vehicle (10 ml/kg bodyweight s.c.) or MTf-hFc-IL-1RA (135 mg/kg s.c.), MTfpep-NIP228-IL-1RA fusion protein (25-100 mg/kg s.c.) or NIP228-IL-1RA (100 mg/kg s.c). Sham operated mice received PBS vehicle (10 ml/kg bodyweight s.c.). Mice were re-tested for changes in mechanical hyperalgesia 4 h post dose as described above. Mice were also re-tested at 1, 2 and 4 days post dose.

Data Analysis

Statistical analysis was performed in GraphPad Prism. Only animals which completed the study were included in the analysis. Results were analysed using 2-way ANOVA. Pairwise comparisons, where appropriate, were made using Tukey's test.

FIG. 1:

Schematic Representation of the Different Molecules Studied.

In A, the constructs with NIP228 hIgG1TM antibody alone and incorporating as genetic fusion with flexible linker or chemical conjugation MTf and MTfpep.

In B, the constructs with NIP228hIgGTM antibody or the Fc fragment of the antibody containing the therapeutic molecule IL-1RA (Kineret) with an analgesic effect and incorporating after genetic fusion MTf or MTfpep with a flexible linker.

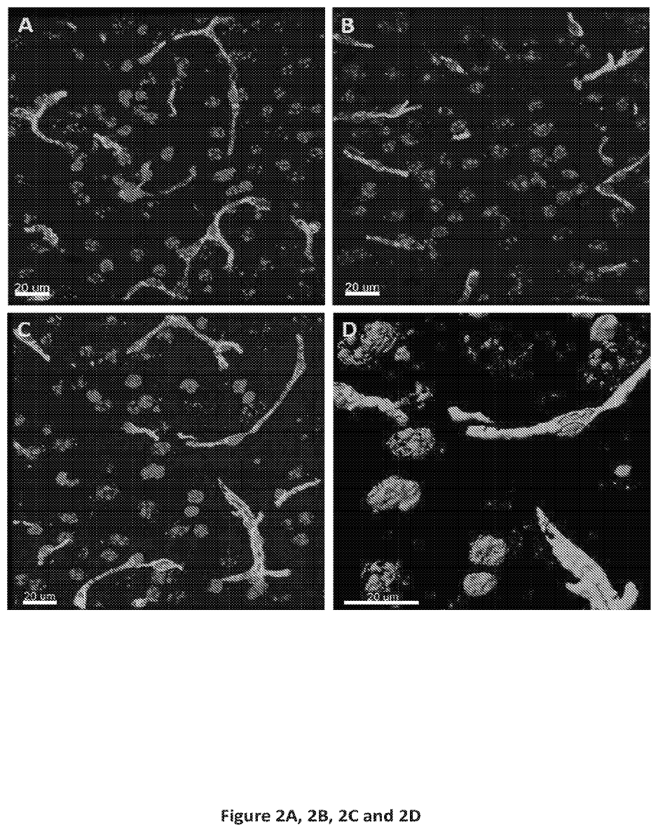

FIG. 2:

Representative 3D Confocal Image of Brain Distribution of Different Constructs Based on the mAb NIP228 an hIgG1TM 2 Hrs after IV Administration in CD-1 Female Mice.

Cell nuclei are blue (DAPI) and capillaries are green (Texas Red). (A) represents the distribution of the NIP228 labelled with Alexa F647 (red); (B) represents the distribution of MTf chemically conjugated to NIP228 labelled with Alexa F647 (red); (C) represents the distribution of MTfpep chemically conjugated to NIP228 labelled with Alexa F647 (red); (D) shows magnified surface rendered (quantified) Texas Red labelled blood capillaries (green) and MTfpep chemically conjugated to NIP228 labelled with Alexa F647 (red). These 3D images demonstrate that the brain parenchyma distribution is much higher for labelled NIP228 incorporating full-length MTf or MTfpep compare to NIP228 alone. See method section for the details of the quantitative procedures. Scale bar represents 20 μm.

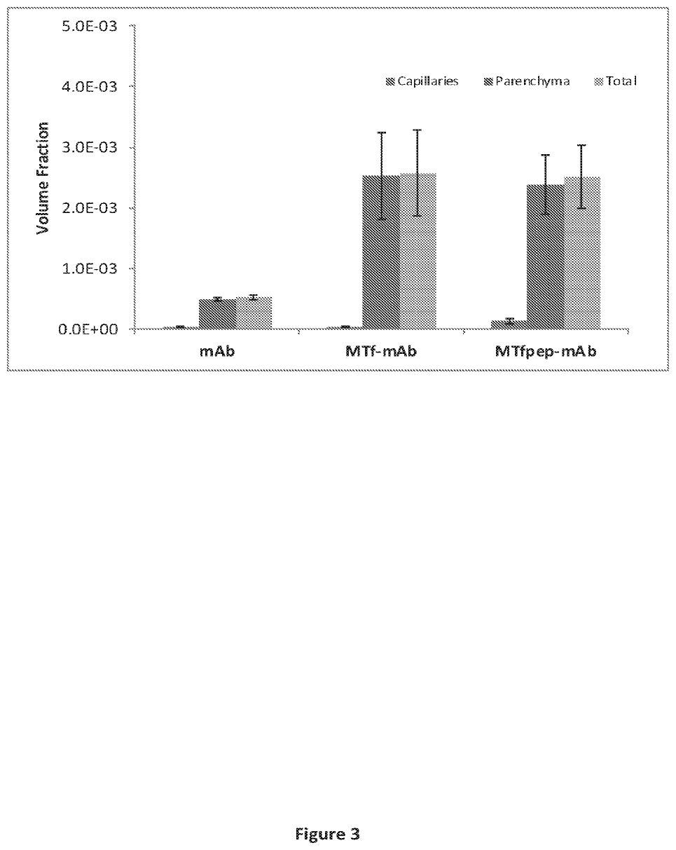

FIG. 3:

Confocal Fluorescence Microscopy Analysis and Semi-Quantification of the Distribution of the Different Molecules in the Brain Parenchyma.

At 2 h post IV injection, greater than 95% of the MTf-mAb or MTfpep-mAb in the brain is found in the parenchyma rather than the capillary blood vessels. The data clearly show that the molecules containing MTf full-length protein or MTfpep are very efficiently transported across the brain capillary endothelium to the brain parenchyma in Balb/c female mice. Data is reported as Mean±Standard Deviation (SD).

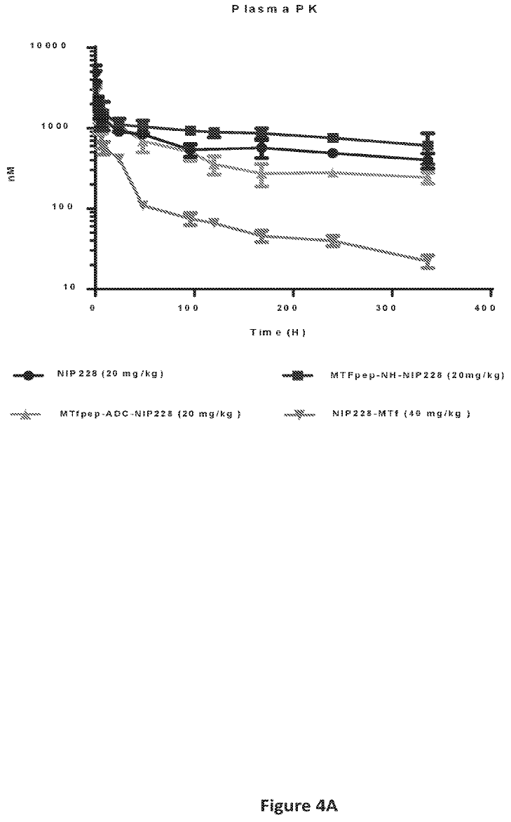

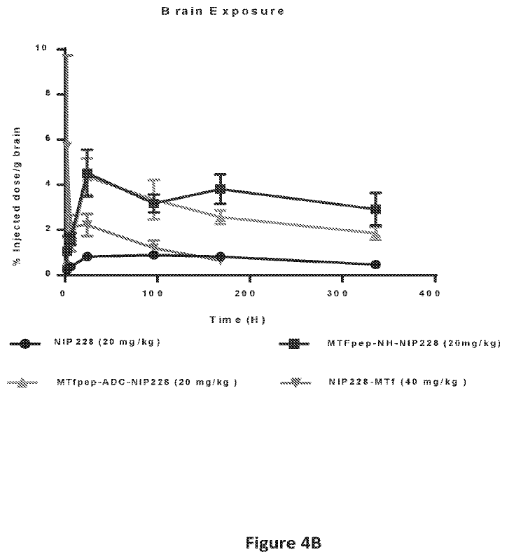

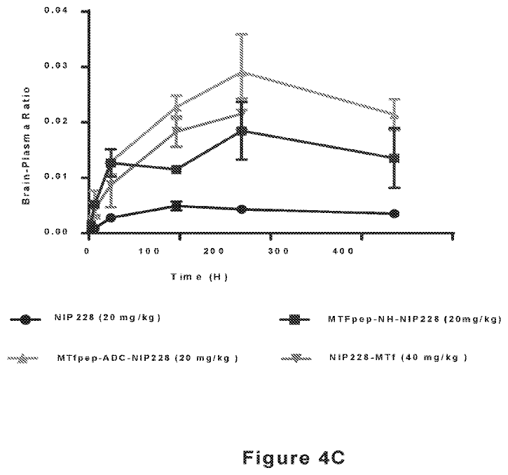

FIG. 4:

Plasma and Brain Exposure of MTf or MTfpep Targeted IgGs in a Mouse PK Assay.

(A) Plasma PK of MTf or MTfpep targeted hIgG1TM compared to a non-targeted isotype control (NIP228) over a two-week period. (B) Brain exposure as a measure of % injected dose per gram of brain. (C) Comparison of brain:plasma ratio. All molecules dosed at the same molar concentration. N=6 per group. PK, pharmacokinetic. S.E.M provided for each measured point indicated.

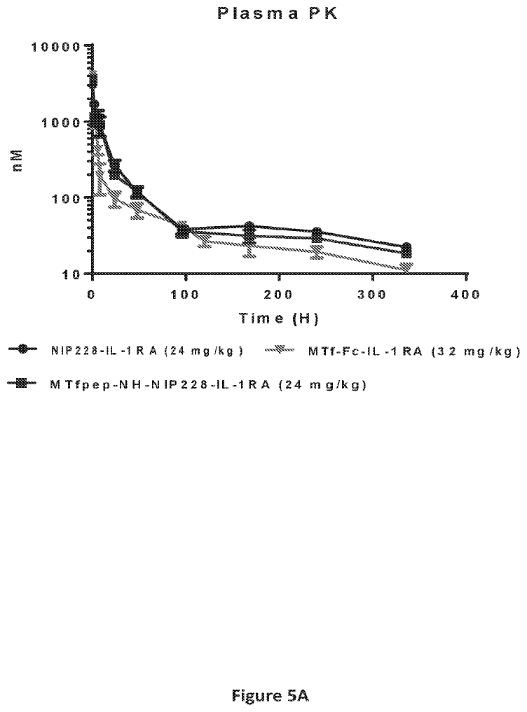

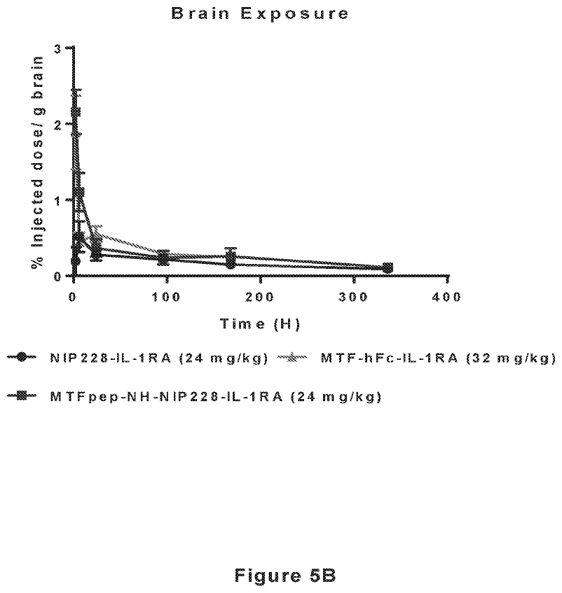

FIG. 5:

Plasma and Brain Exposure of MTf and MTfpep Targeted IgG-IL-1RA Fusion Molecules in a Mouse PK Assay.

(A) Plasma PK of MTf and MTfpep targeted hIgG fused to IL-1RA compared to a non-targeted isotype control (NIP228) over a two-week period. (B) Brain exposure as a measure of % injected dose per gram of brain. All molecules dosed at the same molar concentration. N=6 per group. PK, pharmacokinetic; IL-1RA, interleukin 1 receptor antagonist.

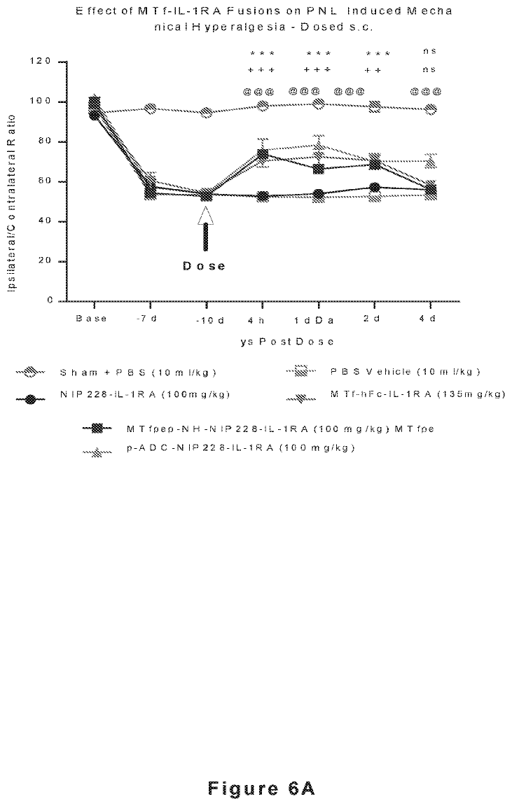

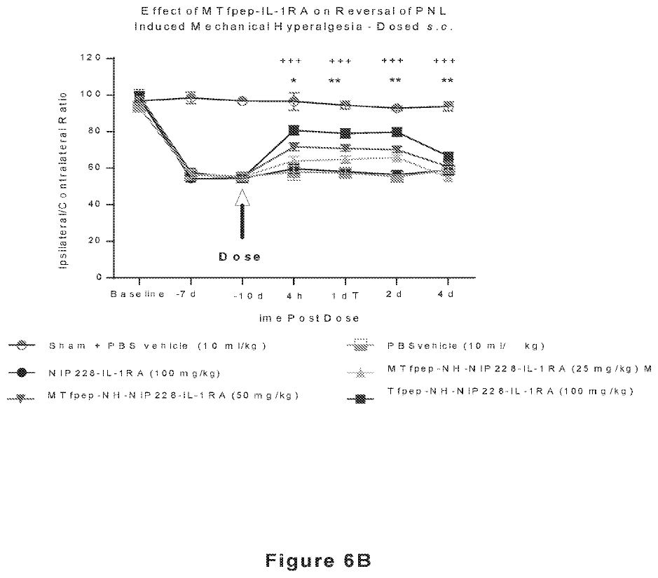

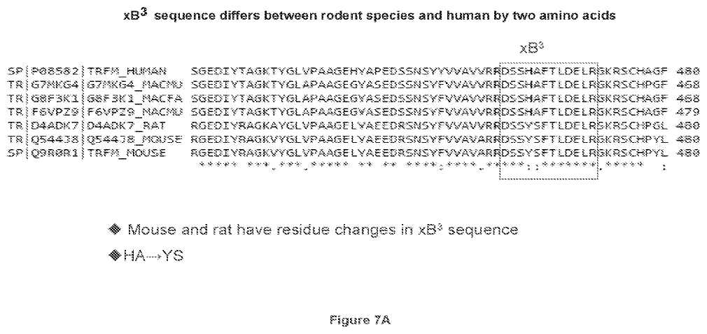

FIG. 6:

The Analgesic Effect of MTf-IL-1RA Fusions on the Mouse Partial Nerve Ligation Model:

(A) Comparison the analgesic effect of IL-1RA fusion constructs containing MTf and MTfpep with isotype control (NIP228), vehicle control and non-ligated (sham operated) control group. N=8 per group. Data analysed using 2-way ANOVA with time and treatment as dependant factors. Subsequent statistical significance obtained using Tukey's Post Hoc test.++P<0.01; +++P<0.001−Op+NIP228-IL-1RA vs Op+MTf-Fc-IL-1RA 135 mg/kg; *** P<0.001−Op+NIP228-IL-1RA vs Op+MTfpep-NH-NIP228-IL-1RA 100 mg/kg; @@@P<0.001−Op+NIP228 vs Op+MTfpep-ADC-NIP228-IL-1RA 100 mg/kg. (B) Dose response of MTfpep targeted NIP228 higG1TM-IL-1RA fusions on the reversal of partial nerve ligation-induced mechanical hyperalgesia. N=7-10 per group. Data analysed using 2 way ANOVA with time and treatment as dependent factors. Subsequent statistical significance obtained using Turkey's Post Hoc test. * P<0.05; ** P<0.01−NIP228 vs MTfpep 50 mg/kg; +++P<0.001−NIP228 vs MTfpep 100 mg/kg

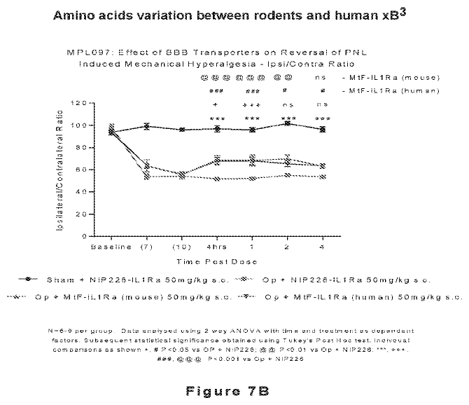

FIG. 7

Sequence Differences in the Amino Acid Sequence of the Peptide of SEQ ID NO: 2 and the Transport of the Peptides Across the BBB.

FIG. 7A shows sequence differences in the amino acid sequence of the peptide of DSSHAFTLDELR (SEQ ID NO: 2) and DSSYSFTLDELR (SEQ ID NO: 3). FIG. 7B shows a comparison of the transport of the peptides across the BBB. Quite surprisingly, the replacement of two amino acids, HA to YS, did not have an observable impact on the ability of the peptide fragment of the present invention, xB³, to cross the BBB.

Results

The MTf BBB Transporting Peptide

The most efficiently transcytosed peptide, DSSHAFTLDELR, (MTfpep or xB³) was previously selected as the candidate for investigating BBB transport in vivo. MTfpep is a 12 amino acid peptide located between amino acid 460 to 471 of full length MTf. This peptide is unique to MTf and is not present in other Tf family members such as Tf, lactotransferrin and ovotransferrin. MTfpep sequence differs between rodent species and human by two amino acids, with histidine and alanine at position 4 and 5 are replaced by tyrosine and serine respectively in the rodent species. The two amino acids variation between rodent and human species have no effect on the ability of MTfpep to cross the brain capillary endothelium, nor the ability to transport a payload into the brain and the subsequent efficacy (data not shown).

In Vivo Brain Distribution Study of MTf and MTfpep Chemically Conjugated to Fluorescently Labeled NIP228

To demonstrate the delivery of an antibody across the BBB in vivo using MTf or MTfpep, wild-type mice were injected with conjugates of the control mAb NIP228-AF647 (FIG. 2A), MTf-NIP228-AF647 (FIG. 2B) or MTfpep-NIP228-AF647 (FIG. 2C) via intravenous injection. After 2 hours post injection, the mice were sacrificed, PBS perfused and brain penetration evaluated and semi-quantified using 3D confocal fluorescence microscopy. Approximately two times greater fractional fluorescence was measured in the brain parenchyma of both MTf-NIP228-AF647 and MTfpep-NIP228-AF647 conjugates compare to NIP228-AF647 alone (FIG. 3). More than 95% of the total AF647 fluorescence signal was localized to the brain parenchyma with the remainder localized to the brain capillaries for both MTf-mAb and MTfpep-mAb conjugates. This first step of analysis and experimentation demonstrated that incorporation of MTf or MTfpep increases the transport of a mAb through the brain capillary endothelium to the brain parenchyma.

Pharmaco-Kinetic (PK) Properties of Fusion and Conjugated Antibodies after Incorporation of MTf or MTfpep

To confirm that MTf and MTfpep possessed improved brain targeting we conducted plasma PK and brain exposure studies in C57BL/6J mice. These studies were performed for a period of 2 weeks, with plasma samples (0.08, 2, 4, 6, 8, 24, 48, 96, 120, 168, 240 & 336 hours) and capillary depleted brain homogenate samples taken at regular intervals (2, 6, 24, 96, 168 & 336 hours) throughout that 2-week period. The serial sampling procedure in this study resulted in composite profiles for plasma exposure following a single intravenous dose of each of the molecules being tested. All molecules were dosed at the same molar equivalent (NIP228, MTfpep-NH-NIP228, MTfpep-ADC-NIP228 were dosed at 20 mg/kg while NIP228-MTf was dosed at 40 mg/kg to compensate for the higher molecular weight of MTf). All parameters were derived using the mean of each data point. FIG. 4A shows the mean plasma exposure profiles (nM+/−average deviation) with Tₘₐₓ achieved at the first-time point (10 min) after intravenous dosing. There is little difference in the plasma PK profiles of the isotype control and the MTfpep constructs, with MTfpep-ADC-NIP228 having a slightly faster clearance value of 14.1 (ml/day/kg) compared to isotype control and the MTfpep genetically fused to NIP228 which possess clearance values of 8 and 5 (ml/day/kg), respectively. However, NIP228-MTf has a significantly altered plasma PK profile, with a much faster clearance rate of 74 (ml/day/kg) resulting in a significantly shorter half-life of 5.5 days and a 3-fold lower area under the curve (AUC) when compared to NIP228 alone control (13.8 days) (Table 1 in suppl mat).

Brain Exposure for Genetically Fused and Chemically Conjugated Antibodies after Incorporation of MTf or MTfpep

Measurement of each of the test molecules in brain homogenate was performed to determine the central exposure. Brain samples were taken from PBS perfused animals at 2, 6, 24, 96, 168 and 336 hours post intravenous administration and processed to homogenate for analysis via MSD assay (FIG. 4B). For both NIP288 and conjugated MTfpep-NIP228 mAbs, Tₘₐₓ occurred at 24 hours post administration, while NIP228-MTf conjugated mAb achieved Tₘₐₓ 2 hours after administration. However, there was a large variation between samples at this time point for NIP228-MTf. NIP228-MTf was below the level of quantification for the assay after 168 hours. NIP228 reached Tₘₐₓ 4 days after administration (FIG. 4B). MTfpep-conjugated to NIP228 had a significantly prolonged brain exposure when compared to NIP228-MTf and NIP228 alone (FIG. 4B), with a peak exposure of about 4% of injected dose at the 24 hour time point. There was also a significantly higher brain-plasma ratio for both MTfpep-NIP228 conjugated mAbs and NIP228-MTf conjugated mAb, peaking at between 1.5-3% at 168 hours post administration compared to peak of 0.5% for NIP228 mAb alone (FIG. 4C).

Pharmaco-Kinetic (PK) Properties for MTf and MTfpep IL-1RA Fusion Proteins

Peripheral PK analysis was performed on MTfpep and MTf protein with C-terminal IL-1RA fusions over a two week period. MTfpep was genetically conjugated with a flexible linker (Gly4Ser) x2 to the N-terminal end of the heavy chain of NIP228 and IL-1RA was conjugated to the C-terminal end of the heavy chain of NIP228 with a (Gly₄Ser) x3 linker. However, MTf protein was genetically conjugated to the N-terminal end of the Fc domain with C-terminal IL-1RA fusion as described above (FIG. 1B).

Plasma samples were taken at regular intervals throughout the two week period and processed as described above. NIP228 and MTfpep-NIP228-IL-1RA fusions demonstrated a 4.5 and 7.2-fold reduction in plasma exposure, respectively, as measured by AUC(ₗₐₛₜ) (FIG. 5A & Table 2 suppl mat) due to a 5-10-fold increased distribution phase (Vz) and 9-15-fold increase in clearance (CL) when compared to the plasma exposure of the mAbs without the presence of IL-1RA (FIG. 4A & Table 1 suppl mat). MTf-Fc-IL-1RA AUC₍ₗₐₛₜ₎ is reduced 2.3-fold compared to reduction observed for NIP228-MTf; (FIG. 5A and Tables 1 & 2 suppl mat), possibly due to higher clearance rates (CL) for all IL-1RA conjugated mAbs (Table 2 suppl mat). Cₘₐₓ for all constructs occurred at the first measured time-point, 10 min post administration.

Brain Exposure for MTf and MTfpep IL-1RA Fusion Proteins

Brain exposure of the IL-1RA fusions was measured as described above and a composite exposure profile generated (FIG. 5B). Tₘₐₓ for each molecule tested was at the first measured time point at (2 h), which is somewhat earlier than that observed for the MTfpep-NIP228 and NIP228 constructs without IL-1RA, which were 24 h and 96 h, respectively (FIG. 4B). Brain exposure of MTfpep-NIP228-IL-1RA and MTf-hFc-IL-1RA was very similar with a maximum exposure of 2.2% injected dose per gram of brain at the first-time point. MTf-hFc-IL-1RA was only detectable in the brain for the first week of the study, after which it was below the lower limit of quantification (LLOQ) for the assay (FIG. 5B). The lower central exposure of the MTfpep and MTf IL-1RA fusion proteins was possibly due to faster peripheral clearance rates (CL) for the IL-1RA fusions as compared to the MTf fusions lacking IL-1RA.

Reversal of Mechanical Hyperalgesia by BBB Transmigrating MTf-IL-1RA Fusions

Neuropathic pain can be induced by partial ligation of the sciatic nerve in laboratory animals as first described by Seltzer et al.³⁰ and adapted for mice by Chessell et al.²⁹. The mechanical hyperalgesia that develops in the model has been shown to be sensitive to the central administration of IL-1RA.²³ MTf-hFc-IL-1RA or MTfpep (NH or ADC)-NIP228-IL-1RA were administered s.c. at 135 mg/kg or 100 mg/kg, respectively (equimolar dosing) to test whether mAb-IL-1RA fusions were able to reverse the mechanical hyperalgesia and the mice monitored for 4 days post dose. No difference in effects was noted between, vehicle, PBS and control mAb-IL-1RA (NIP228-IL-1RA) treated mice indicating NIP228-IL-1RA was not able to access IL-1 receptors in the central compartment (FIG. 6A). Administration of MTf-hFc-IL-1RA and MTfpep-NIP228-IL-1RA fusions resulted in reversal of mechanical hyperalgesia at time points from 4 h to 2 days post dose. Only MTfpep (ADC)-NIP228-IL-1RA achieved reversal at 4 days post dose. The magnitude of the response was very similar for both MTf-hFc-IL-1RA and MTfpep-IL-1RA fusions tested (FIG. 6A).

To investigate the relationship between dose and the reversal of mechanical hyperalgesia, we administered MTfpep-NH-NIP228-IL-1RA at doses 25, 50 and 100 mg/kg. The different dose levels demonstrated differences in magnitude of the response, but not duration. 50 and 100 mg/kg dosing of MTfpep-NH-NIP228-IL-1RA produced statistically significant analgesic effect compared to NIP228-IL-1RA as analysed using Turkey's Post Hoc Test (FIG. 6B).

TABLE 1
Plasma pharmacokinetic analysis of the different constructs based on a monoclonal antibody NIP228
hIgG1TM. The design of the molecules analysed are represented schematically in FIG. 1A.

| | | Terminal | | | | | | |
|---|---|---|---|---|---|---|---|---|
| | IV dose | half-life | Tₘₐₓ | Cₘₐₓ | AUCₗₐₛₜ | AUC(_{0 to inf}) | Vz | CL |
| Dose group | (mg/kg) | (days) | (day) | (μg/mL) | (day · μg/mL) | (day · μg/mL) | (ml/kg) | (ml/day/kg) |
| NIP228 hIgG1TM | 20 | 13.8 | 0.00333 | 735 | 1300 | 2490 | 159 | 8.02 |
| MTfpep-ADC- | 20 | 8.6 | 0.00333 | 426 | 972 | 1420 | 174 | 14.1 |
| NIP228 hIgG1TM | | | | | | | | |
| MTfpep-NH- | 20 | 15.9 | 0.00333 | 543 | 1860 | 3940 | 116 | 5.07 |
| NIP228 hIgG1TM | | | | | | | | |
| NIP228-MTf | 40 | 5.5 | 0.00333 | 793 | 488 | 541 | 590 | 73.9 |
| hIgG1TM | | | | | | | | |

Tₘₐₓ = Time to reach maximum or peak concentration; Cₘₐₓ = The maximum or peak concentration; AUCₗₐₛₜ = Area Under the Curve of Plasma concentration at the last timepoint; Vz = terminal volume of distribution; CL = systemic clearance.

TABLE 2
Plasma pharmacokinetic analysis of the different constructs based
on a monoclonal antibody NIP228 hIgG1TM - IL-1RA. The design of
the molecules analysed are represented schematically in FIG. 1B.

| | | Terminal | | | | | |
|---|---|---|---|---|---|---|---|
| | IV dose | half-life | Tₘₐₓ | Cₘₐₓ | AUCₗₐₛₜ | Vz | CL |
| Dose group | (mg/kg) | (days) | (day) | (μg/mL) | (day · μg/mL) | (ml/kg) | (ml/day/kg) |
| MTfpep-NIP228- | 24 | 10.6 | 0.00708 | 665 | 260 | 1180 | 77 |
| IgG1TM IL-IRA | | | | | | | |
| NIP228 hIgG1TM | 24 | 7.5 | 0.00708 | 557 | 286 | 786 | 73 |
| IL-IRA | | | | | | | |
| MTf-Fc-IL-IRA | 32 | 7.2 | 0.00708 | 985 | 217 | 1360 | 130 |

Tₘₐₓ = Time to reach maximum or peak concentration; Cₘₐₓ = The maximum or peak concentration; AUCₗₐₛₜ = Area Under the Curve of Plasma concentration at the last timepoint; Vz = terminal volume of distribution; CL = systemic clearance.

Terminal elimination half-life (t½) was calculated using regression of the concentration data including at least the last three sampling time points with measurable concentration of for all dosing cohorts:

$${t}^{1 / 2} = ln ( 2 ) / λ z$$

where λz was the first-order terminal rate constant estimated via linear regression of the terminal log-linear decay phase.
The plasma clearance after IV administration (CL) was estimated as:

$$CL = Dose / {AUC}_{inf}$$

where, AUC extrapolated to infinity (AUC_{inf}) was calculated by trapezoidal method. The volume of distribution after IV administration (Vz) was estimated as:

$$Vz = Dose / λ z AUCinf IV$$

The steady state volume of distribution after administration (Vss) was estimated as:

$$Vss = MRT \times CL$$

Discussion

These examples demonstrate that both melanotransferrin (MTf) and a 12 amino-acid peptide derived from MTf (MTfpep) when incorporated in the sequence of a control antibody (NIP228) by chemical conjugation or by genetic fusion can induce the transport of the antibody across the brain capillary endothelium to reach therapeutic concentration in the brain parenchyma. Multiple techniques were used to confirm the brain penetration of antibodies modified with MTf or MTf peptide. Using confocal fluorescence microscopy, it was possible to visualise an antibody conjugated to MTf or MTfpep labelled with a fluorescent marker, Alexa F647, in the brain parenchyma. Following i.v. administration in mice the MTf or MTfpep fusions showed a significant increased brain distribution compared to the unconjugated antibody, with 95% of all fluorescence localized in the brain parenchyma (FIGS. 2 and 3). This observation was confirmed by analysing the concentration of antibodies incorporating MTf and MTfpep in brain homogenate from animals dosed i.v. (FIGS. 4B and 4C). In this study MTf was genetically fused to the C-terminus of the antibody heavy chain, and the MTfpep was attached to the antibody either by genetic fusion to the N-terminus of the antibody heavy chain, or by chemical conjugation to the thiol group of an unpaired cysteine introduced into the Fc domain of the antibody. The designs of the different molecules analyzed are shown in FIG. 1. Brain exposure of the different molecules after i.v. administration was determined following extensive PBS perfusion of the animals, followed by brain homogenisation. Homogenate was depleted of capillaries in order to determine more accurate amount of molecules present in the brain parenchyma. Molecules containing MTfpep have a significantly prolonged brain exposure when compared to either the MTf containing molecule or the antibody alone (FIG. 4B). A peak exposure of about 4% of injected dose at the 24 hour time point and between 3-4% of injected dose for a period of 2 weeks was observed. This was also reflected in the brain-plasma ratio for both MTfpep- or MTf-NIP228 conjugated or fused proteins compared to NIP228 control antibody alone (FIG. 4C). Brain-plasma ratio peaks at about 1.5-3% at 168 hours post administration for the MTfpep or MTf containing proteins compared to 0.5% peak ratio for NIP228 mAb (FIG. 4C). This study also investigated the plasma PK profiles of these antibodies (FIG. 4A). The presence of MTfpep on the heavy chains of the antibodies does not affect its plasma PK in contrast to the mAbs containing the MTf protein, which shows a 3-fold shorter peripheral half-life (Table 1 suppl mat). This is most likely due to a higher tissue distribution of this construct due to the MTf protein.

To further validate the apparent ability of MTf and MTfpep to deliver molecules into the CNS, pharmacodynamic studies were performed in a mouse model of neuropathic pain. Fusion to IL-1RA enabled measurement of analgesia through a reduction in mechanical hypersensitivity following partial sciatic nerve ligation.²³ The addition of IL-1RA significantly altered the PK of these molecules resulting in a reduction in plasma exposure compared to the equivalent molecule lacking IL-1RA (FIG. 5A & Table 2 suppl mat). This was due to a much-increased distribution phase (Vz) and increase in clearance (CL) (FIG. 4A & Table 1 suppl mat), presumably due to IL-1 receptor mediated clearance. Brain exposure for the IL-1RA fusions was also altered (FIG. 5B). A peak of exposure is seen at 2 h, which is much earlier than that observed for the MTfpep-NIP228 and NIP228 constructs without IL-1RA (FIG. 4B). Brain exposure was similar with 2.2% injected dose per g brain extract at the first-time point. The lower central exposure of the MTfpep and MTf IL-1RA proteins was likely due to faster peripheral clearance. In the neuropathic pain model, the control antibody (NIP288) lacking either IL-1RA, MTf, or MTfpep did not induce analgesia (FIG. 6A). The fusions containing both IL-1RA and either MTf or MTfpep resulted in reversal of mechanical hyperalgesia at time points from 4 h to 2 days post dose. Only MTfpep chemically conjugated to NIP228-IL-1RA had reversal at 4 days post dose. The magnitude of the response was very similar for all MTf-IL-1RA fusions tested (FIG. 6A). It is of significance to note that the reversal of hyperalgesia was 2-4 days in length, one day more than if IL-1RA is directly injected intrathecally where the effect lasted 1 day.²³ This effect was also found to be dose dependent (FIG. 6B). In addition, we have shown here that our peptide of 12 amino-acid originating from MTf is delivering an analgesic compound (IL-1RA) in the CNS which shows better plasma and brain pharmacokinetic properties than a construct²³ targeting the transferrin receptor by an antibody. Whilst the dose required to achieve analgesia is high, in the absence of more potent analgesics, IL-1RA has enabled this research demonstrating BBB penetration and a pharmacological effect. Future research building on these findings, applying the BBB technology to more efficacious molecules, will assist in the quest to find treatments for neuropathic pain.

Citations for the references noted in the above examples are as follows:
13. Yang J, Tiong J, Kennard M, et al. Deletion of the GPI pre-anchor sequence in human p97—A general approach for generating the soluble form of GPI-linked proteins. Protein Expr Purif 2004; 34: 28-48.
19. Karkan D, Pfeifer C, Vitalis T Z, et al. A unique carrier for delivery of therapeutic compounds beyond the blood-brain barrier. PLoS One; 3. Epub ahead of print 2008. DOI: 10.1371/journal.pone.0002469.
21 Eyford B A, Abraham T, Munro L, et al. A peptide vector delivers siRNA across the blood-brain barrier resulting in attenuation of ischemic stroke. In revision: Nature Biomed Eng. 2017
22. Hegedus D D, Pfeifer T a, Theilmann D a, et al. Differences in the expression and localization of human melanotransferrin in lepidopteran and dipteran insect cell lines. Protein Expr Purif 1999; 15: 296-307.
23. Webster C I, Hatcher J, Burrell M, et al. Enhanced delivery of IL-1 receptor antagonist to the central nervous system as a novel anti-transferrin receptor-Il-1RA fusion reverses neuropathic mechanical hypersensitivity. Pain 2017; 158(4): 660-668
24. Persic L, Roberts A, Wilton J, et al. An integrated vector system for the eukaryotic expression of antibodies or their fragments after selection from phage display libraries. Gene 1997; 187: 9-18.
25. Oganesyan V, Gao C, Shirinian L, et al. Structural characterization of a human Fc fragment engineered for lack of effector functions. Acta Crystallogr Sect D Biol Crystallogr 2008; 64: 700-704.
26. Daramola O, Stevenson J, Dean G, et al. A high-yielding CHO transient system: Coexpression of genes encoding EBNA-1 and GS enhances transient protein expression. Biotechnol Prog 2014; 30: 132-141.
27. Pace C N, Vajdos F, Fee L, et al. How to measure and predict the molar absorption coefficient of a protein. Protein Sci 1995; 4: 2411-2423.
28. Thompson P, Fleming R, Bezabeh B, et al. Rational design, biophysical and biological characterization of site-specific antibody-tubulysin conjugates with improved stability, efficacy and pharmacokinetics. J Control Release 2016; 236: 100-116.
29. Chessell I P, Hatcher J P, Bountra C, et al. Disruption of the P2X7 purinoceptor gene abolishes chronic inflammatory and neuropathic pain. Pain 2005; 114: 386-396.
30. Seltzer Z, Dubner R, Shir Y. A novel behavioral model of neuropathic pain disorders produced in rats by partial sciatic nerve injury. Pain 1990; 43: 205-218.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, it is intended in accordance with the present invention that combination therapy using a glutamate modulating agent and an immunotherapeutic agent can be employed to treat cancers other than the specific cancers disclosed in the description and Examples herein. Further, glutamate modulating agents and immunotherapeutic agents other than those disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination. For instance, in the area of cancer treatment, CD3 binding moieties of the present invention, with or without linkers of the type described herein, may be bound to immunology targeting anti-cancer agents including other types of antibodies, e.g., polyclonal antibodies, antibody fragments, peptides, proteins, small molecules, adjuvants, cytokines, oncolytic viruses, vaccines, bi-specific molecules and cellular therapeutic agents.

1. A method for treating a first interleukin-1 (IL-1) related disease in the brain of a subject by delivering a therapeutic payload across the blood-brain barrier of the subject, comprising administering to the subject said therapeutic payload, wherein said therapeutic payload comprises
(i) an IL-1 receptor atagonist;
(ii) a p97 fragment comprising an amino acid sequence at least 80% identical to DSSHAFTLDELR (SEQ ID NO: 2) coupled to the IL-1 receptor atagonist.

2. The method of claim 1 which further comprises treating a second IL-1 related disease other than in the brain of the subject.

3. The method of claim 2 wherein the therapeutic payload is administered to the subject other than intracranially.

4. The method of claim 2 wherein the therapeutic payload is administered by oral, intravenous, intramuscular, subcutaneous, injection or infusion.

5. The method of claim 1 wherein the first IL-1 related disease is selected from a group comprising autoinflammatory diseases, autoimmune disease, metabolic syndromes, chronic inflammation and malignancy, infectious diseases, ischemic diseases, fatigues associated with inflammatory and non-inflammatory diseases, neuropathic pain, fabry disease, epilepsy and seizure, fabrile disease, fabrile infection-related epilepsy syndrome, fabrile status epilepticus, generalised epilepsy with febrile seizures plus, dravet syndrome, Idiopathic hemiconvulsion hemiplegia and epilepsy syndrome, Parkinson's disease, IL-1 receptor antagonist deficiency syndrome, and new-onset refractory status epilepticus.

6. The method of claim 2 wherein the second IL-1 related disease is selected from a group comprising autoinflammatory diseases, autoimmune disease, metabolic syndromes, chronic inflammation and malignancy, infectious diseases, ischemic diseases, fatigues associated with inflammatory and non-inflammatory diseases, neuropathic pain, fabry disease, epilepsy and seizure, fabrile disease, fabrile infection-related epilepsy syndrome, fabrile status epilepticus, generalised epilepsy with febrile seizures plus, dravet syndrome, Idiopathic hemiconvulsion hemiplegia and epilepsy syndrome, Parkinson's disease, IL-1 receptor antagonist deficiency syndrome, and new-onset refractory status epilepticus.


SEQUENCE LISTING (3 sequences)

SEQ ID NO: 1 - protein, 738 aa, Homo sapiens
MRGPSGALWLLLALRTVLGGMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGAAAPAPGAPLLPLLLPALAARLLPPAL

SEQ ID NO: 2 - protein, 12 aa, Homo sapiens
DSSHAFTLDELR

SEQ ID NO: 3 - protein, 12 aa, Mus musculus
DSSYSFTLDELR